(19) United States
(12) Patent Application Publication
(10) Pub. No.: US 2013/0323259 A1
(43) Pub. Date: Dec. 5, 2013

(54) IL-21 LIGANDS

(71) Applicants: Lars Anders Svensson, Malmoe (SE); Mette Dahl Andersen, Vaerloese (DK); Jens Breinholt, Dyssegaard (DK); Charlotte Wiberg, Bjaerred (SE); Berit Olsen Krogh, Roedovre (DK); Dorthe Lundsgaard, Vaerloese (DK); Hanne Benedicte Rasmussen, Hilleroed (DK)

(72) Inventors: Lars Anders Svensson, Malmoe (SE); Mette Dahl Andersen, Vaerloese (DK); Jens Breinholt, Dyssegaard (DK); Charlotte Wiberg, Bjaerred (SE); Berit Olsen Krogh, Roedovre (DK); Dorthe Lundsgaard, Vaerloese (DK); Hanne Benedicte Rasmussen, Hilleroed (DK)

(73) Assignee: Novo Nordisk A/S, Bagsvaerd (DK)

(21) Appl. No.: 13/979,430

(22) PCT Filed: Jan. 17, 2012
(86) PCT No.: PCT/EP2012/050633

§ 371 Date: Aug. 21, 2013

Related U.S. Application Data
(60) Provisional application No. 61/434,989, filed on Jan. 21, 2011.
(60) Provisional application No. 61/493,002, filed on Jun. 3, 2011.

(30) Foreign Application Priority Data
Jan. 17, 2011 (EP) 11151073.1
May 31, 2011 (EP) 11168328.0
Oct. 31, 2011 (EP) 11187212.3

(51) Int. Cl. C07K 16/24

(52) U.S. Cl. CPC C07K 16/244
USPC 424/139.1; 530/387.9; 530/387.3

(57) ABSTRACT

The present invention relates to IL-21 ligands binding to a discontinuous epitope on the surface of the IL-21 molecule as well as use thereof.

The present invention is concerned with a discontinuous epitope present on IL-21, and ligands which bind to this epitope.

IL-21 is a type I cytokine, which exerts pleiotropic effects on both innate and adaptive immune responses. It is mainly produced by activated CD4+ T cells, follicular T cells and Natural killer cells. In addition, recent evidence suggests that Th17 cells can produce large amounts of IL-21.

IL-21 increases the cytotoxicity of CD8+ T cells and can promote proliferation of CD8+ cells in the presence of antigens. IL-21 is induced by IL-6, a cytokine known to promote development of Th17 cells. IL-21 acts on T helper cells in an autocrine manner promoting its own production and supporting differentiation of T-helper cells into Th17 cells. In agreement with this, IL-21 deficient mice show an impaired Th17 response. IL-21 also acts on B-cells and increases antibody production; however, IL-21 is not essential for production of functional antibodies, whereas IL-21Rα negative mice exhibit both reduced proliferation as well as impaired cytotoxicity of CD8+ cells. A recent set of studies suggests that IL-21 produced by CD4+ cells is critical for the ability of CD8+ T cells to control viral infection.

The ability of IL-21 to augment immunity has spurred substantial interest in the therapeutic use of IL-21. It is currently evaluated in clinical trials against metastatic melanoma types and renal cancer. Animal studies have demonstrated a synergistic effect between IL-21 and tumor specific antibodies, which could suggest a future therapeutic use of IL-21 as a potentiator of anti-tumor antibodies. Furthermore, IL-21 plays a complex role in autoimmune diseases. The ability of IL-21 to downregulate IgE production suggests that it could be used therapeutically against asthma and allergy. Results from animal studies support this view. On the other hand, the ability of IL-21 to promote Th17 development makes it a pro-inflammatory cytokine and a number of different IL-21 and IL-21Rα inhibitors are currently investigated for potential use in treatment of a range of different autoimmune diseases.

IL-21 has a four helix bundle structure, arranged in an up-up-down-down topology typical for the class I cytokines. IL-21 signals through a heterodimeric receptor complex consisting of the private chain IL-21Rα and the common yC chain the latter being shared by IL-2, IL-4, IL-7, IL-9, and IL-15. The IL-21Rα chain binds IL-21 with high affinity and provides the majority of the binding energy. However, interaction with the γC chain is required for signaling and IL-21 mutants which bind IL-21Rα but fail to interact properly with γC are potent antagonists of IL-21 signaling. Both IL-2 and IL-15 employ a third receptor chain, IL-2Rα and IL-15Rα respectively. These receptors are expendable for signaling, but works as high affinity component of the receptor complex capturing IL-2 or IL-15 and present it to IL-2Rβ after which recruitment of γC takes place.

Monoclonal antibodies specific for IL-21 are known in the art, for example from WO2007111714 and WO2010055366 (Zymo-Genetics, Inc.). In particular, WO2010055366 describes an anti-IL-21 antibody, designated by clone number 362.78.1.44, which has a high affinity for its cognate antigen, and other desirable properties, showing specificity for human and cynomolgus monkey IL-21. In WO2010055366, clone 362.78.1.44 is characterised as binding to a discontinuous epitope on human IL-21, which comprises amino acids from two peptides spanning residues Ile45 to Leu56 and Glu129 to Leu144 of the IL-21 sequence set forth as SEQ ID No. 2 in WO2010055366.

### SUMMARY OF THE INVENTION

We define herein a novel epitope of IL-21 which is characteristically bound by higher-affinity ligands. Binding of a ligand to this epitope stabilises helix C of human IL-21 (hIL-21). This leads to a general stabilisation of IL-21, which is thought to promote high-affinity binding and/or efficient inhibition of IL-21 induced effects.

The epitope of the invention is bound by the natural IL-21 receptor, IL-21Rα (SEQ ID No. 14). We identify the binding interfaces between IL-21 and IL-21Rα which are responsible for the interaction of the two molecules, and thus a target for antibodies which are designed to inhibit the activity of IL-21 through disruption of the interaction between IL-21 with its cognate receptor.

We also describe ligands, such as antibodies, which bind specifically to the epitope according to the invention, as well as methods for making and using such ligands.

### DETAILED DESCRIPTION OF THE INVENTION

Unless otherwise stated, all technical and scientific terms used herein have the same meanings as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

### BRIEF DESCRIPTION OF THE DRAWINGS

FIG. 1: The sequences referred to herein.

FIG. 2: HX monitored by mass spectrometry identifies regions of hIL-21 involved in NNC 0114-0005 and NNC 0114-0019 binding. (A) Mass/charge spectra corresponding to the peptide fragment 29-44, MQGQDRHMIRMRQLID (m/z=676.68, z=3) situated in helix A. (B) Mass/charge spectra corresponding to the peptide fragment 93-98, ERIINV (m/z=743.47, z=1) situated in helix C. For all spectra the upper panels show the non-deuterated controls, second panel shows the peptide after 100 sec in-exchange with D₂O, third and bottom panel shows the peptide after 100 sec in-exchange in the presence of NNC 0114-0005 or NNC 0114-0019, respectively.

FIG. 3: Hydrogen exchange time-plots of representative peptides of hIL-21 in the absence or presence of NNC 0114-0005 or NNC 0114-0019. Deuterium incorporation (Da) of hIL-21 peptides is plotted against time on a logarithmic scale in the absence (▪) or presence of NNC 0114-0005 (x) or NNC 0114-0019 (□). Peptide Q30-M39 represent a region of hIL-21 helix A where both NNC 0114-0005 and NNC 0114-0019 binds. Peptide E93-V98 represent a region of hIL-21 helix C where only NNC 0114-0005, and not NNC 0114-0019, bind. Peptides R40-N51 and F136-S162 represent regions of hIL-21 where neither NNC 0114-0005 nor NNC 0114-0019 bind. However, a marked stabilization of the hIL-21 structure is observed when NNC 0114-0005, but not NNC 0114-0019, binds to hIL-21 as displayed by marked decrease of deuterium exchange in the long time points above 300 sec.

FIG. 4: Sequence coverage of HX analyzed peptides of hIL-21 in the presence and absence of NNC 0114-0005. The primary sequence is displayed above the HX analyzed peptides (shown as horizontal bars). Peptides showing similar exchange patterns both in the presence and absence of NNC 0114-0005 are displayed in white whereas peptides showing reduced deuterium incorporation upon NNC 0114-0005 binding are coloured black. Boxed sequence regions define the epitope.

FIG. 5: Sequence coverage of HX analyzed peptides of hIL-21 in the presence and absence of NNC 0114-0019. The primary sequence is displayed above the HX analyzed peptides (shown as horizontal bars). Peptides showing similar exchange patterns both in the presence and absence of NNC 0114-0019 are displayed in white whereas peptides showing reduced deuterium incorporation upon NNC 0114-0019 binding are coloured black. Boxed sequence regions define the epitope.

FIG. 6: Hydrogen exchange time-plots of representative peptides of hIL-21 in the absence or presence of 0114-0005, -0038, -0039, -0040, -0041 or -0042. Deuterium incorporation (Da) of hIL-21 peptides is plotted against time on a logarithmic scale in the absence (▪, ▴) or presence of -0005 (x), -0038 (□), -0039 (▭), -0040 (+), -0041 (◯) or -0042 (−). Peptide M29-D44 represents a region of hIL-21 helix A where 0114-0005, -0038, -0039, -0040, -0041 and -0042 bind. Peptide 145-N51 represent a region of hIL-21 where binding of 0114-0005, -0038, -0039, -0040, -0041 and -0042 bind can be seen as a very weak deuterium exchange difference in the early timepoints. Furthermore, marked stabilization of the hIL-21 structure upon 0114-0005, -0038, -0039, -0040, -0041 or -0042 binding to hIL-21 is observed by a noticeable decrease of deuterium exchange in the long time points above 300 sec. Peptide E93-V98 represents a region of hIL-21 helix C where 0114-0005, -0038, -0039, -0040, -0041 and -0042 bind. Peptide E138-S162 represents a region of hIL-21 where none of the mAbs bind. However, stabilization of the hIL-21 structure is observed when 0114-0005, -0038, -0039, -0040, -0041 or -0042 bind to hIL-21 as displayed by a decrease of deuterium exchange in the long time points above 300 sec.

FIG. 7: Sequence coverage of HX analyzed peptides of hIL-21 in the presence and absence of 0114-0005, -0038, -0039, -0040, -0041 and -0042. The primary sequence is displayed above the HX analyzed peptides (shown as horizontal bars). All peptides displayed similar exchange behaviour upon binding of any of the mAbs examined. Peptides showing similar exchange patterns in the early timepoints both in the presence and absence of 0114-0005, -0038, -0039, -0040, -0041 or -0042 are displayed in white whereas peptides showing reduced deuterium incorporation upon 0114-0005, -0038, -0039, -0040, -0041 or -0042 binding are coloured black.

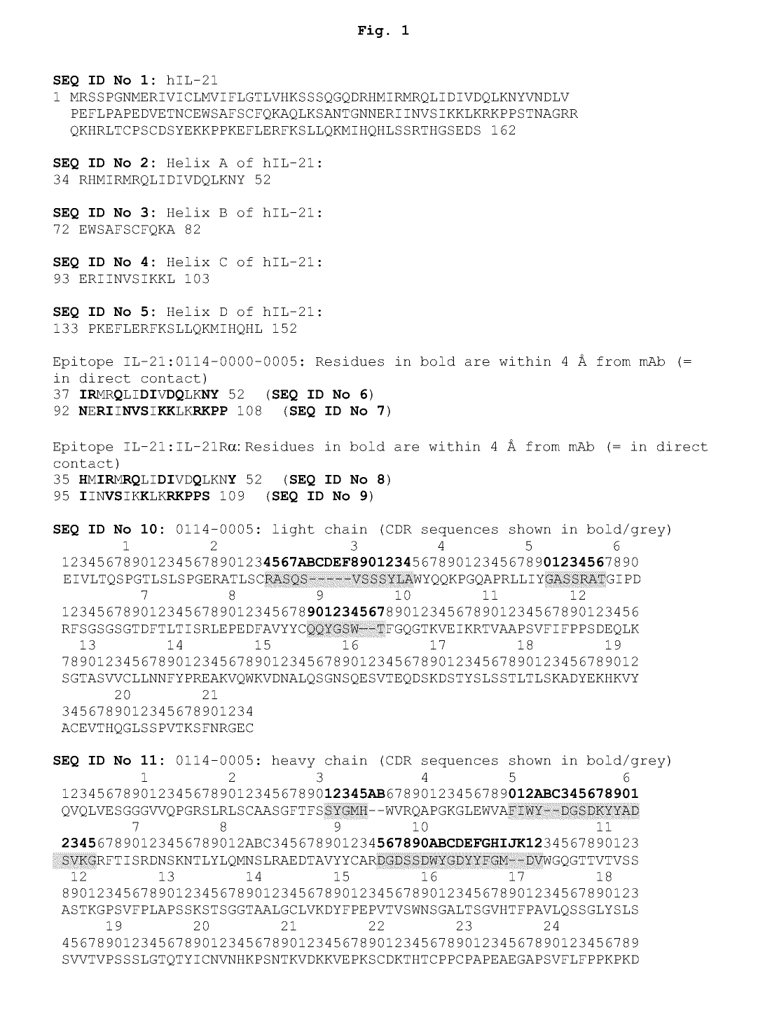

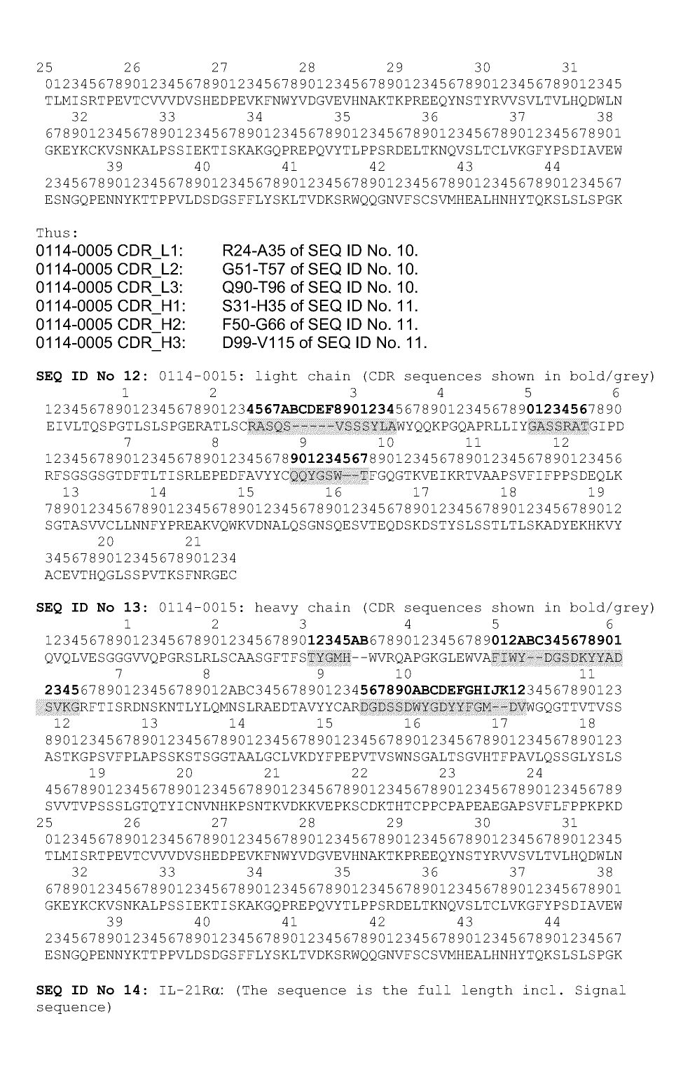

### DEFINITIONS

The term “treatment”, as used herein, refers to the medical therapy of any human or other animal subject in need thereof. Said subject is expected to have undergone physical examination by a medical or veterinary medical practitioner, who has given a tentative or definitive diagnosis which would indicate that the use of said specific treatment is beneficial to the health of said human or other animal subject. The timing and purpose of said treatment may vary from one individual to another, according to the status quo of the subject's health. Thus, said treatment may be prophylactic, palliative, symptomatic and/or curative.

In terms of the present invention, prophylactic, palliative, symptomatic and/or curative treatments may represent separate aspects of the invention.

The epitope of hIL-21 which is bound by antibody NNC 0114-0005 has been defined by X-ray crystallography and HX-MS and is described herein.

In this context, specific binding is binding which occurs, in the case of immunoglobulin molecules, as a result interaction between an immunoglobulin binding site formed by at least one CDR which is part of an immunoglobulin variable domain and the epitope. Preferably, specific binding occurs as a result of interaction between one or more, such as two or three, CDRs in an immunoglobulin light chain and one or more, such as two or three, CDRs in an immunoglobulin heavy chain which form an immunoglobulin binding site, and the epitope.

Specific binding may also be characterized as binding which occurs with a given binding affinity. For example, the binding constant is preferably of the order of 1 μM or less, for example, 100 nM, 10 nM, 1 nM, 100 pM, 1 pM, or less.

A “discontinuous” epitope is an epitope which is formed by two or more separated regions of a polypeptide which are not adjacent to each other in the linear peptide sequence, but which are arranged in the three-dimensional structure of the polypeptide to form an epitope.

As used herein, an “isolated” compound is a compound that has been removed from its natural environment. “A purified” compound is a compound that has been increased in purity, such that it exists in a form that is more pure than it exists in its natural environment.

### Antibodies

Preferred ligands, useful in the present invention, are based on immunoglobulins. Immunoglobulins suitable in the present invention include antibodies and T-cell receptor (TCR) molecules.

The term “antibody”, “monoclonal antibody” and “mAb” as used herein, is intended to refer to immunoglobulin molecules and fragments thereof according to the invention that have the ability to specifically bind to an antigen. Full-length antibodies comprise four polypeptide chains, two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Variable regions and CDRs in an antibody sequence may be identified by aligning the sequences against a database of known variable regions (frameworks and CDRs are defined according to the Kabat numbering scheme herein—(Kabat, E A, Wu, T T, Perry, H M, et al. Sequences of Proteins of Immunological Interest, Fifth Edition. US Department of Health and Human Services, Public Health Service, National Institutes of Health, NIH Publication No. 91-3242, 1991).

The fragment crystallizable region (“Fc region”/“Fc domain”) of an antibody is the tail region of an antibody that interacts with cell surface receptors called Fc receptors and some proteins of the complement system. This property allows antibodies to activate the immune system. The Fc domain can, however, comprise amino acid mutations that result in modification of these effector functions. Preferably, a modified Fc domain comprises one or more, preferably all of the following mutations that will result in decreased affinity to certain Fc receptors (L234A, L235E, and G237A) and in reduced Clq-mediated complement fixation (A3305 and P331S), respectively (residue numbering according to the EU index). Such Fc domains will still retain a long in vivo half life.

The other part of an antibody, called the “Fab region”/“Fab domain”/“Fab fragment”, contains variable sections that define the specific target that the antibody can bind. These fragments can be produced from intact antibodies using well known methods, for example by proteolytic cleavage with enzymes such as papain (to produce Fab fragments) or pepsin (to produce F(ab′)₂ fragments). Alternatively, antibody fragments may be produced recombinantly, using standard recombinant DNA and protein expression technologies.

Examples of binding fragments encompassed within the term “antibody” thus include but are not limited to: (i) a Fab fragment, a monovalent fragment consisting of the VL, VH, CL and CH I domains; (ii) F(ab)₂ and F(ab′)2 fragments, a bivalent fragment comprising two Fab fragments linked by a disulfide bridge at the hinge region; (iii) a Fd fragment consisting of the VH and CH1 domains; (iv) a scFv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426: and Huston et al. (1988) Proc. Natl. Acad. Sci. USA 85:5879-5883). Such single chain antibodies are also intended to be encompassed within the term “antibody”. Other forms of single chain antibodies, such as diabodies are also encompassed wihin the term “antibody”. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Hol-liger, P., et al. (1993) Proc. Natl. Acad. Sci. USA 90:6444-6448; Poljak, R. J., et al. (1994) Structure 2:1121-1123).

It is understood that the antigen may have one or more antigenic determinants (epitopes) comprising (1) peptide antigenic determinants which consist of single peptide chains, (2) conformational antigenic determinants which consist of more than one spatially contiguous peptide chains whose respective amino acid sequences are located disjointedly along polypeptide sequence; and (3) post-translational antigenic determinants which consist, either in whole or part, of molecular structures covalently attached to the antigen after translation, such as carbohydrate groups, or the like.

The terms “human antibody”, “human antibodies”, as used herein, means antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3.

Antibodies according to the present invention, in which CDR sequences derived from antibodies originating from another mammalian species, such as a mouse, have been grafted onto human framework sequences and optionally potentially further engineered by mutagenesis are referred to as “humanized antibodies”.

The term “chimeric antibody” or “chimeric antibodies” refers to antibodies according to the invention whose light and heavy chain genes have been constructed, typically by genetic engineering, from immunoglobulin variable and constant region genes belonging to different species. For example, the variable segments of genes from a mouse monoclonal antibody may be joined to human constant segments.

### Epitopes

The term “epitope”, as used herein, is defined in the context of a molecular interaction between an “antigen binding polypeptide”, such as an antibody (Ab), and its corresponding antigen (Ag). Generally, “epitope” refers to the area or region on an Ag to which an Ab specifically binds, i.e. the area or region in physical contact with the Ab. Physical contact may be defined through various criteria (e.g. a distance cut-off of 2-6 Å, such as 3 Å, such as 4 Å, such as 5 Å; or solvent accessibility) for atoms in the Ab and Ag molecules. A protein epitope may comprise amino acid residues in the Ag that are directly involved in binding to a Ab (also called the immunodominant component of the epitope) and other amino acid residues, which are not directly involved in binding, such as amino acid residues of the Ag which are effectively blocked by the Ab, i.e. amino acid residues within the “solvent-excluded surface” and/or the “footprint” of the Ab.

The epitope for a given antibody (Ab)/antigen (Ag) pair can be described and characterized at different levels of detail using a variety of experimental and computational epitope mapping methods. The experimental methods include mutagenesis, X-ray crystallography, Nuclear Magnetic Resonance (NMR) spectroscopy, Hydrogen deuterium eXchange Mass Spectrometry (HX-MS) and various competition binding methods; methods that are known in the art. As each method relies on a unique principle, the description of an epitope is intimately linked to the method by which it has been determined. Thus, depending on the epitope mapping method employed, the epitope for a given Ab/Ag pair may be described differently.

Antibodies of the present invention may be described or specified in terms of the epitope(s) or portion(s) of an IL-21 protein that they recognize or specifically bind. The epitope(s) or the polypeptide portions(s) may be specified as e.g. by N-terminal and C-terminal positions, or by size in contiguous amino acid residues. Antibodies of the present invention may also be described or specified in terms of their cross-reactivity. Antibodies that do not bind any other analog, ortholog, or homolog of a polypeptide are included.

Antibodies that bind to the same antigen can be characterised with respect to their ability to bind to their common antigen simultaneously and may be subjected to “competition binding”/“binning”. In the present context, the term “binning” refers to a method of grouping antibodies that bind to the same antigen. “Binning” of antibodies may be based on competition binding of two antibodies to their common antigen in assays based on standard techniques such as surface plasmon resonance (SPR), ELISA or flow cytometry.

A “bin” is defined using a reference antibody. If a second antibody is unable to bind to the antigen at the same time as the reference antibody, the second antibody is said to belong to the same “bin” as the reference antibody, In this case the reference and the second antibody are competing for binding to the antigen, thus the pair of antibodies is termed “competing antibodies”. If a second antibody is capable of binding to the antigen at the same time as the reference antibody, the second antibody is said to belong to a separate “bin”. In this case the reference and the second antibody are not competing for binding to the antigen, thus the pair of antibodies is termed “non-competing antibodies”.

Antibody “binning” does not provide direct information about the epitope. Competing antibodies, i.e. antibodies belonging to the same “bin” may have identical epitopes, overlapping epitopes or even separate epitopes. The latter is the case if the reference antibody bound to its epitope on the antigen takes up the space required for the second antibody to contact its epitiope on the antigen (“steric hindrance”). Non-competing antibodies generally have separate epitopes.

The term “affinity”, as used herein, defines the strength of the binding of an antibody to an epitope. The affinity of an antibody is measured by the equilibrium dissociation constant KD, defined as [Ab]×[Ag]/[Ab−Ag] where [Ab−Ag] is the molar concentration of the antibody-antigen complex, [Ab] is the molar concentration of the unbound antibody and [Ag] is the molar concentration of the unbound antigen at equilibrium. KD can also be described from the kinetics of complex formation and dissociation, determined by e.g. the SPR method. The rate constants corresponding to the association and the dissociation of a monovalent complex are referred to as the association rate constant ka (or kₒₙ) and dissociation rate constant kd (or k_{off}), respectively. KD is then related to ka and kd through the equation KD=kd/ka. The affinity constant KA is defined by 1/KD. Preferred methods for determining antibody specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589-601 (1983).

The antibody or fragment thereof may be modified in order to increase its serum half-life, for example, by conjugating with molecules—such as fatty acids or fatty acid derivates, PEG (poly ethylene glycol) or other water soluble polymers, including polysaccharide polymers and peptide derived polymers to increase the half-life.

### Structure of Ligands

As described above, a ligand as referred to herein may be an antibody (for example IgG, IgM, IgA, IgA, IgE) or fragment (for example Fab, Fv, disulphide linked Fv, scFv, diabody) which comprises at least one heavy and a light chain variable domain which are complementary to one another and thus can associate with one another to form a VH/VL pair. It may be derived from any species naturally producing an antibody, or created by recombinant DNA technology; or isolated from sequence libraries using molecular display technologies, such as phage display, whether library sequences isolated from serum, B-cells, hybridomas, transfectomas, yeast or bacteria.

In a preferred embodiment of the invention the ligand comprises at least one single heavy chain variable domain of an antibody and one single light chain variable domain of an antibody such that the two regions are capable of associating to form a complementary VH/VL pair.

Where V-gene repertoires are used variation in polypeptide sequence is preferably located within the structural loops of the variable domains. The polypeptide sequences of either variable domain may be altered by DNA shuffling or by mutation in order to enhance the interaction of each variable domain with its complementary pair. In a preferred embodiment of the invention the ligand is a single chain Fv fragment. In an alternative embodiment of the invention, the ligand consists of a Fab region of an antibody.

In a further aspect, the present invention provides nucleic acid encoding at least a ligand as herein defined. Thus in a further aspect, the present invention provides a vector comprising nucleic acid encoding at least ligand as herein defined.

### Therapeutic Applications

IL-21 is involved in T-cell mediated immunity, and has been shown to promote a number of inflammatory cytokines. Accordingly, the ligands according to invention can be used in the treatment of diseases involving an inappropriate or undesired immune response (immunological disorders), such as inflammation, autoimmunity, conditions involving such mechanisms as well as graft vs. host disease. In one embodiment, such disease or disorder is an autoimmune and/or inflammatory disease. Examples of such autoimmune and/or inflammatory diseases are Systemic Lupus Erythematosus (SLE), Rheumatoid Arthritis (RA) and inflammatory bowel disease (IBD) (including ulcerative colitis (UC) and Crohn's disease (CD)), multiple sclerosis (MS), scleroderma and type 1 diabetes (T1 D), and other diseases and disorders, such as PV (pemphigus vulgaris), psoriasis, atopic dermatitis, celiac disease, kol, hashimoto's thyroiditis, graves' disease (thyroid), Sjogren's syndrome, guillain-barre syndrome, goodpasture's syndrome, additon's disease, Wegener's granulomatosis, primary biliary sclerosis, sclerosing cholangitis, autoimmune hepatitis, polymyalgia rheumatica, paynaud's phenomenon, temporal arteritis, giant cell arteritis, autoimmune hemolytic anemia, pernicious anemia, polyarteritis nodosa, behcet's disease, primary bilary cirrhosis, uveitis, myocarditis, rheumatic fever, ankylosing spondylitis, glomerulenephritis, sarcoidosis, dermatomyositis, myasthenia gravis, polymyositis, alopecia greata, type I diabetes, Colitis-Associated Tumorigenesis and vitilgo. Other examples can be found in PCT application WO 01/46420, which is directed at the use of IL-17 for treatment of autoimmune and/or inflammatory diseases and wherein several examples of such diseases are given, and WO2010/055366, the contents of which are specifically incorporated herein be reference.

In one embodiment, such disease or disorder is SLE, RA or IBD. In one embodiment, such disease or disorder is MS.

The IL-21 ligands of the present invention may be administered in combination with other medicaments as is known in the art.

### Pharmaceutical Compositions

The present invention further includes pharmaceutical compositions/formulations, comprising a pharmaceutically acceptable carrier and a polypeptide/ligand/antibody according to the invention as well as kits comprising such compositions. The pharmaceutical composition according to the invention may be in the form of an aqueous formulation or a dry formulation that is reconstituted in water/an aqueous buffer prior to administration.

Pharmaceutical compositions comprising ligands/antibodies/polypeptides according to the invention may be supplied as a kit comprising a container that comprises the compound according to the invention. Therapeutic polypeptides can be provided in the form of an injectable solution for single or multiple doses, or as a sterile powder that will be reconstituted before injection. Pharmaceutical compositions comprising compounds according to the invention are suitable for subcutaneous and/or IV administration.

Moreover, there is provided a method for treating a condition associated with an aberrant immune response, comprising administering to a subject a therapeutically effective amount of a ligand identifiable using an assay method as described above.

Amino acids I37 to Y52 and N92 to P108 define a novel epitope on human IL-21. We have determined that high affinity ligands which bind to IL-21 are more likely to make contacts with IL-21 which fall within the recited amino acid ranges. By binding to this epitope, ligands have a stabilising effect on IL-21. In particular, the highly mobile helix C is stabilised in the bound state, greatly reducing the energy present in the IL-21 molecule in the free state.

Moreover, we have determined that the epitope is specifically bound by monoclonal antibody NNC 0114-0005 (may also be referred to as e.g. “0005” herein). The sequences of the light and heavy chain variable domains are set forth in SEQ ID No 10 and SEQ ID No 11, respectively. This antibody is the same antibody which is disclosed in WO2010/055366, designated therein by hybridoma clone number 362.78.1.44. This antibody may also be referred to in several ways herein: “0005”, “NNC-0000-0005”, “0006”, and “NNC-0000-0006”. However, in WO2010/055366, the epitope targeted by the same antibody is incorrectly defined to comprise residues Ile45 to Leu56, and Glu129 to Leu144. We have now shown that this is incorrect.

Similarly, the epitope defined herein is also bound by monoclonal antibody NNC 0114-0015 (may also be referred to as ‘0015” herein), which is disclosed in WO2010/055366, designated hybridoma clone number 362.597.3.15. The sequences of the light and heavy chain variable domains are set forth in SEQ ID 1N No 12 and SEQ ID No 13, respectively. NNC 0114-0015 has a single amino acid change, HC S31T (numbering according to Kabat—Kabat, E A, Wu, T T, Perry, H M, et al. Sequences of Proteins of Immunological Interest, Fifth Edition. US Department of Health and Human Services, Public Health Service, National Institutes of Health, NIH Publication No. 91-3242, 1991), relative to NNC 0114-0005.

In a first aspect, there is provided a ligand, preferably an antibody, which binds specifically to an epitope defined herein, provided that the ligand is not naturally occurring IL-21Rα (SEQ ID No. 14 sets forth the complete IL-21Rα sequence including the signal sequence which is not part of the mature IL-21Rα protein or any previously described IL-21R variants comprising the binding site for IL-21 as described herein), or the monoclonal antibodies NNC 0114-0005 or NNC 0114-0015, the light and heavy chains of which are set forth in SEQ ID No. 10 and SEQ ID No. 11 and SEQ ID No. 12 and SEQ ID No. 13 respectively, herein.

Preferably, the ligand is an immunoglobulin, such as an antibody or an antibody fragment, or a TCR (T-cell receptor) or fragment thereof.

We have shown that ligands which bind the recited epitope stabilise IL-21, by stabilising helix C, and the entire molecule. It is believed that stabilisation of the IL-21 molecule increases the affinity of the binding of the ligands, since the bound IL-21 achieves a very low energy state compared to unbound molecules.

In another aspect, or embodiment of the present invention, a ligand is an antibody or antibody fragment, wherein the heavy chain comprises at least one of CDR1, CDR2 or CDR3 as set forth in SEQ ID No. 11, and the light chain comprises at least one of CDR1, CDR2 or CDR3 as set forth in SEQ ID No. 10. The CDRs of SEQ ID No. 10 and 11 as listed are defined according to the Kabat scheme (Kabat, E A, Wu, T T, Perry, H M, et al. Sequences of Proteins of Immunological Interest, Fifth Edition. US Department of Health and Human Services, Public Health Service, National Institutes of Health, NIH Publication No. 91-3242, 1991).

In one embodiment, the light chain comprises the sequence set forth in SEQ ID No. 10.

In one embodiment, the heavy chain comprises the sequence set forth in SEQ ID No. 11.

The ligand may moreover comprise an Fc, which mediates antibody effector functions.

In another aspect, there is provided a ligand, preferably an antibody, which binds specifically to an IL-21 epitope at the interface which is formed between IL-21 and IL-21Rα, identified herein. Preferably, the ligand binds at an epitope which encompasses any one or more of the following residues: R34, R38, Q41, K102 and R105. Preferably, the ligand binds to at least R34 and K102; or R34 and R105; or R34, K102, and R105; or R38 and K102; or R38 and R105; or R38, K₁₀₂, and R105; or Q41 and K102; or Q41 and R105; or Q41, K102, and R105; or R105, R34, and R38; or R105, R34, R38, and Q41; or K102, R34, and Q41; or: R34, R38, Q41, K102 and R105; or R38, Q41, K102 and R105; or R34, Q41, K102 and R105; or R34, R38, K102 and R105.

Also encompassed are anti-IL-21Rα ligands which bind to IL-21Rα at the IL-21:IL-21R binding interface identified herein.

In another aspect, there is provided a ligand, preferably an antibody, which binds specifically to the discontinuous epitope of IL-21 according to the invention, provided that the ligand is not: (i) naturally occurring IL-21Rα (SEQ ID No. 14), (ii) the monoclonal antibody NNC 0114-0005, the light and heavy chains of which are set forth in SEQ ID No. 10 and SEQ ID No. 11 respectively, and (iii) the monoclonal antibody NNC 0114-0015, the light and heavy chains of which are set forth in SEQ ID No. 12 and SEQ ID No. 13 respectively. The present invention thus comprises any ligand having such properties, except the following compounds: IL-21Rα, monoclonal antibody NNC 0114-0005, and monoclonal antibody NNC 0114-0015.

In another aspect, there is provided a ligand, preferably an antibody, which binds to a discontinuous epitope on IL-21, wherein said epitope comprises at least one of amino acids 137 to Y52 and at least one of amino acids N92 to P108 of IL-21, as set forth in SEQ ID No.1, provided that the ligand is not: (i) naturally occurring IL-21Rα (SEQ ID No. 14), (ii) the monoclonal antibody NNC 0114-0005, the light and heavy chains of which are set forth in SEQ ID No. 10 and SEQ ID No. 11 respectively, and (iii) the monoclonal antibody NNC 0114-0015, the light and heavy chains of which are set forth in SEQ ID No. 12 and SEQ ID No. 13 respectively. The present invention thus comprises any ligand having such properties, except the following compounds: IL-21Rα, monoclonal antibody NNC 0114-0005, and monoclonal antibody NNC 0114-0015.

In one embodiment, the ligand according to the invention binds to an epitope comprising amino acids I37 to Y52 and N92 to P108 of IL-21 as set forth in SEQ ID No. 1.

In another aspect or embodiment of the present invention, a ligand comprises a light chain comprising at least one of CDR1, CDR2 or CDR3 corresponding to the residues listed below according to SEQ ID No. 10, and a heavy chain comprising at least one of CDR1, CDR2 or CDR3 corresponding to the residues listed below according to SEQ ID No. 11.

0114-0005 CDR_L1: R24-A35 of SEQ ID No. 10.

0114-0005 CDR_L2: G51-T57 of SEQ ID No. 10.

0114-0005 CDR_L3: Q90-T96 of SEQ ID No. 10.

0114-0005 CDR_H1: S31-H35 of SEQ ID No. 11.

0114-0005 CDR_H2: F50-G66 of SEQ ID No. 11.

0114-0005 CDR_H3: D99-V115 of SEQ ID No. 11.

In another embodiment, the ligand according to the invention comprises a light chain comprising at least one of CDR1 and CDR3 as set forth in SEQ ID No. 10, and a heavy chain comprising at least one of CDR2 and CDR3 as set forth in SEQ ID No. 11

In another aspect, there is provided a ligand, preferably an antibody, which binds to IL-21 at the binding interface between IL-21 and IL-21Rα, wherein said ligand binds to an epitope which includes at least one, preferably at least two, preferably at least three, preferably at least four of R34, R38, Q41, R105, and K102 in the sequence of IL-21 set forth in SEQ ID No 1, provided that the ligand is not: (i) naturally occurring IL-21Rα (SEQ ID No. 14), (ii) the monoclonal antibody NNC 0114-0005, the light and heavy chains of which are set forth in SEQ ID No. 10 and SEQ ID No. 11 respectively, and (iii) the monoclonal antibody NNC 0114-0015, the light and heavy chains of which are set forth in SEQ ID No. 12 and SEQ ID No. 13 respectively. The present invention thus comprises any ligand having such properties, except the following compounds: IL-21Rα, monoclonal antibody NNC 0114-0005, and monoclonal antibody NNC 0114-0015.

In one embodiment, the ligand according to the invention binds to an epitope on IL-21 comprising R34, R38, Q41, R105, and K102.

In another embodiment, the ligand according to the invention interferes with the binding of IL-21 to IL-21Rα.

In another embodiment, the KD of the interaction of human IL-21 with the ligand according to the invention is 10⁻¹² (M) or less. In another embodiment, the ligand according to the invention is preferably an antibody, wherein said antibody binds to R34, R38, Q41, R105, and K₁₀₂ in the sequence of IL-21 set forth in SEQ ID NO 1, and wherein the KD of the interaction of human IL-21 with the antibody is 10⁻¹² (M) or less.

In another embodiment, the ligand according to the invention is an antibody, wherein said antibody comprises the CDR3 amino acid sequence as set forth in SEQ ID NO 10 and the CDR3 amino acid sequence as set forth in SEQ ID NO 11.

In another embodiment, the ligand according to the invetion is an antibody, wherein the KD of the interaction of human IL-21 with said antibody is 10⁻¹² (M) or less, wherein said antibody binds to R34, R38, Q41, R105, and K102 in the sequence of IL-21 set forth in SEQ ID NO 1, and wherein said antibody competes with IL-21 for binding to IL-21R.

In another embodiment, the ligand according to the invention is an antibody, such as e.g. an antibody, a monoclonal antibody, a monovalent antibody, or a divalent antibody.

In another aspect or embodiment according to the invention, the ligand is an antibody that is a variant of the monoclonal antibody NNC 0114-0005, the light and heavy chains thereof which are set forth in SEQ ID No. 10 and SEQ ID No. 11 respectively, wherein said ligand comprises one or more mutations in the CDR sequences of SEQ ID No. 10 and/or SEQ ID No. 11, wherein said mutations are selected from one or more from the list consisting of: CDR H1 S31A, CDR H2 Y53F, CDR H2 A61S, CDR H2 S63T, CDR H2S63A, CDR H2 K65R, CDR L1 R24K, CDR L1 S26T, CDR L1 S31T, CDR L1 S31A, CDR L2 S53T, CDR L2 S52A, CDR L2 S54A, CDR L2 S54T, and CDR L2 R55K. It is, of course, understood that such variant antibodies can only comprise one mutation at a given position.

In another aspect, there is provided a pharmaceutical composition comprising a ligand, preferably an antibody, according to the invention and optionally one or more pharmaceutically acceptable excipients/carriers. Such excipients/carriers are well known in the art. Such pharmaceutical compositions are preferably intended for IV administration and/or subcutaneous administration.

In another aspect, there is provided use of a ligand, preferably an antibody, according to the invention for treating an immunological disorder. The immunological disorder is preferably an autoimmune disease.

In a final aspect, there is provided a method of treating an immunological disorder, wherein said method comprises administering to a person in need thereof an appropriate dosis of a ligand, preferably an antibody, according to the invention. In one embodiment, the ligand according to the invention can be used for reducing B cell differentiation in the treatment of autoimmune diseases.

Ligands in accordance with the present invention are suitable for treating conditions which involve an aberrant immune response, including autoimmune diseases. IL-21 is indicated in a number of T-cell specific interactions, and is an immunostimulatory cytokine. Accordingly, there is provided an IL-21-specific ligand as described herein for use in the treatment of a condition involving an aberrant immune response. Moreover, there is provided a method of treating a condition involving an aberrant immune response, comprising to administering to a subject in need thereof a ligand in accordance with the present invention.

The provision herein of the detailed 3-dimensional strutural knowledge of the IL-21:IL-21Rα and the Fab NNC 0114-0009:IL-21 complex, including their binding interface, can form the basis for rationally designing variants of the interacting molecules with desired properties. Properties that might be desirable to improve for antibodies may be chemical or physical properties e.g. solubility, viscosity and stability. Other properties that might be desirable to modulate are the antigenic properties of the antibodies and their ability to be bound by anti-antibodies and to induce effector functions.

It is understood that different aspects and embodiments according to the present invention can be combined.

### EXAMPLES

### Example 1

### Crystal Structure of hIL-21 in Complex with Anti-IL-21 Fab

The 3-dimensional structure of hIL-21 in complex with a Fab fragment, NNC 0114-0009, of the human anti-IL-21 monoclonal antibody NNC 0114-0005 (this Fab fragment may also herein be referred to as e.g. “Fab 0114-0005”) was solved and refined to 1.75 Å resolution using X-ray crystallography. The results demonstrate that the epitope on hIL-21 has an extensive overlap with the binding site for the private hIL-21 receptor chain (IL21Rα). Thus, by virtue of its high affinity, the anti-IL-21 mAb efficiently blocks the binding of hIL-21 to IL21Rα, and, hence, inhibits the biological effects mediated by hIL-21 through its cognate receptor.

hIL-21 (residues 30-162 of SEQ ID NO:1) and anti-IL-21 Fab NNC 0114-0009 (comprising a light chain corresponding to SEQ ID NO: 16 and a heavy chain fragment corresponding to residues 1-234 of SEQ ID NO: 17) were mixed with a slight molar excess of hIL-21 and the complex was purified using size exclusion chromatography. The complex was then concentrated to about 10.3 mg/ml. Crystals were grown with the hanging drop-technique in 25% w/v PEG 3350, 0.1M Citric Acid, pH 3.5, mixed in a ratio of 1:2 (precipitant solution volume:protein solution volume). Total drop size was 3.0 μl. A crystal was prepared for cryo-freezing by transferring 3 μl of a cryo-solution containing 75% of the precipitant solution and 25% glycerol to the drop containing the crystal, and soaking was allowed for about 15 seconds. The crystal was then flash frozen in liquid N₂ and kept at a temperature of 100 K during data collection by a cryogenic N₂ gas stream. Crystallographic data were collected, originally to 2.03 Å resolution at a Rigaku 007HF rotating anode source and thereafter, using the same crystal, to 1.75 Å resolution at beam-line BL911-2 [1] at MAX-lab, Lund, Sweden. Space group determination, integration and scaling of the data were made by the XDS software package [2] and further checked by the Pointless software [3]. Cell parameters for the synchrotron data were determined to be 40.7, 133.3, 53.4 Å, 90°, 106.87° and 90°, respectively, and the space group P2₁. R-sym to 1.75 Å resolution was 5.1% and completeness 98.1%. Molecular replacement, using the Phaser software program [4;5] of the CCP4 suite [6] was used for structure determination. A Fab molecule from the Protein Data Bank (PDB)-deposited [7] structure 3 KDM [8] and a hIL-21 molecule from an earlier determined X-ray crystallographic structure of an IL-21/IL-21Rα complex structure (Example 2) were used for structure determination.

The Fab molecule was divided into two parts, containing the variable and the constant domains, respectively. The two parts were each used as search models in the molecular replacement calculations. For IL-21 the whole molecule, with the exception of undetermined flexible loops, was used as search model. The software ARP/wARP [9] was subsequently used for an initial round if model building and was followed by crystallographic refinements, using the software programs Refmac5 [10] of the CCP4 software package and Phenix.refine [11] of the Phenix software package [12] and by computer graphics inspection of the electron density maps, model corrections and building using the Coot software program [13]. The procedure was cycled until no further significant improvements could be made to the model. Final R- and R-free for all data were 0.191 and 0.241, respectively, and the model showed a root-mean-square deviation (RMSD) from ideal bond lengths of 0.023 Å. The header of the PDB-file from the final refinement cycle is shown I Table 1.

### Results

Anti-IL-21 Fab Effectively Blocks IL-21Rα Binding to the hIL-21 Molecule.

Calculation of the areas excluded in pair-wise interactions by the software program Areaimol (B. Lee and F. M. Richards, J. Mol. Biol., 55, 379-400 (1971) E. B. Saff and A. B. J. Kuijlaars, The Mathematical Intelligences, 19, 5-11 (1997)) of the CCP4 program suite [6] gave for the hIL-21/anti-IL-21 Fab molecular complex of the crystal structure 1189 for hIL-21 and 1084 Å² for anti-IL-21, respectively. The average area excluded in pair-wise interaction between IL-21 molecule and anti-IL-21 Fab was calculated to be 1136 Å².

The direct contacts between the hIL-21 and anti-IL-21 Fab were identified by running the CONTACT software of the CCP4 program suite [6] using a cut-off distance of 4.0 Å between the anti-IL-21 Fab and the hIL-21 molecules. The results from the hIL-21/anti-IL-21 Fab complex crystal structure are shown in Table 2. The resulting hIL-21 epitope for anti-IL-21 was found to comprise the following residues of hIL-21 (SEQ ID NO: 1): Ile 37, Arg 38, Gln 41, Asp 44, Ile 45, Asp 47, Gln 48, Asn 51, Tyr 52, Asn 92, Arg 94, Ile 95, Asn 97, Val 98, Val 98, Ser 99, Lys 101, Lys 102, Arg 105, Lys 106, Pro 107 and Pro 108.

Thus, the anti-IL-21 hIL-21 epitope comprise residues of helix A and C. Additionally, several contact residues (Arg 105 to Pro 108) were identified in the loop segment proceeding helix C. These contact areas agreed very well with what have been determined as the binding site for IL-21Rα on hIL-21 (Example 2).

The anti-IL-21 paratope for hIL-21 included residues Glu 1, Gln 27, Ser 28, Val 29, Ser 30, Ser 32, Tyr 33, Gln 91, Tyr 92, Gly 93, Ser 94 and Trp 95 of the light (L) chain (SEQ ID NO: 16, Table 2), and residues Trp 47, Trp 52, Ser 56, Asp 57, Tyr 59, Tyr 60, Asp 99, Asp 101, Ser 102, Ser 103, Asp 104, Trp 105, Tyr 106, Gly 107, Asp 108, Tyr 109 and Phe 111 of the heavy (H) chain (SEQ ID NO: 17, Table 2). The hIL-21 epitope, and the residues involved in hydrogen-binding, are also indicated in the amino-acid sequence of hIL-21 in Table 2.

TABLE 1
Results from the X-ray model refinement to the observed data of the IL-21/anti-
IL-21 Fab complex by the software program Refmac5 [10] of the CCP4 program software
package [6].

| REMARK | 3 | REFINEMENT. | | | |
|---|---|---|---|---|---|
| REMARK | 3 | PROGRAM : REFMAC 5.6.0085 | | | |
| REMARK | 3 | AUTHORS : MURSHUDOV, VAGIN, DODSON | | | |
| REMARK | 3 | | | | |
| REMARK | 3 | REFINEMENT TARGET : MAXIMUM LIKELIHOOD | | | |
| REMARK | 3 | | | | |
| REMARK | 3 | DATA USED IN REFINEMENT. | | | |
| REMARK | 3 | RESOLUTION RANGE HIGH | (ANGSTROMS) | : | 1.75 |
| REMARK | 3 | RESOLUTION RANGE LOW | (ANGSTROMS) | : | 28.21 |
| REMARK | 3 | DATA CUTOFF | (SIGMA(F)) | : | NONE |
| REMARK | 3 | COMPLETENESS FOR RANGE | (%) | : | 98.26 |
| REMARK | 3 | NUMBER OF REFLECTIONS | | : | 51090 |
| REMARK | 3 | | | | |

| REMARK | 3 | FIT TO DATA USED IN REFINEMENT. | | | |
|---|---|---|---|---|---|
| REMARK | 3 | CROSS-VALIDATION METHOD | | : | THROUGHOUT |
| REMARK | 3 | FREE R VALUE TEST SET SELECTION | | : | RANDOM |
| REMARK | 3 | R VALUE | (WORKING + TEST SET) | : | 0.19342 |
| REMARK | 3 | R VALUE | (WORKING SET) : 0.19089 | | |

| REMARK | 3 | FREE R VALUE | | : | 0.24019 |
|---|---|---|---|---|---|
| REMARK | 3 | FREE R VALUE TEST SET SIZE | (%) | : | 5.0 |

| | | | | |
|---|---|---|---|---|
| REMARK | 3 | FREE R VALUE TEST SET COUNT | : | 2710 |
| REMARK | 3 | | | |

| REMARK | 3 | FIT IN THE HIGHEST RESOLUTION BIN. | | | |
|---|---|---|---|---|---|
| REMARK | 3 | TOTAL NUMBER OF BINS USED | | : | 20 |
| REMARK | 3 | BIN RESOLUTION RANGE HIGH | | : | 1.750 |
| REMARK | 3 | BIN RESOLUTION RANGE LOW | | : | 1.795 |
| REMARK | 3 | REFLECTION IN BIN | (WORKING SET) | : | 3784 |
| REMARK | 3 | BIN COMPLETENESS | (WORKING + TEST) (%) | : | 98.49 |
| REMARK | 3 | BIN R VALUE | (WORKING SET) | : | 0.340 |

| | | | | |
|---|---|---|---|---|
| REMARK | 3 | BIN FREE R VALUE SET COUNT | : | 182 |
| REMARK | 3 | BIN FREE R VALUE | : | 0.345 |
| REMARK | 3 | | | |

| REMARK | 3 | NUMBER OF NON-HYDROGEN ATOMS USED IN REFINEMENT. | | |
|---|---|---|---|---|
| REMARK | 3 | ALL ATOMS : 4655 | | |
| REMARK | 3 | | | |
| REMARK | 3 | B VALUES. | | |
| REMARK | 3 | FROM WILSON PLOT | (A**2) | : NULL |
| REMARK | 3 | MEAN B VALUE | (OVERALL, A**2) | : 30.687 |

| REMARK | 3 | OVERALL ANISOTROPIC B VALUE. | |
|---|---|---|---|
| REMARK | 3 | B11 (A**2) : | 1.22 |
| REMARK | 3 | B22 (A**2) : | −0.87 |
| REMARK | 3 | B33 (A**2) : | −0.96 |
| REMARK | 3 | B12 (A**2) : | 0.00 |
| REMARK | 3 | B13 (A**2) : | −1.05 |
| REMARK | 3 | B23 (A**2) : | 0.00 |
| REMARK | 3 | | |

| REMARK | 3 | ESTIMATED OVERALL COORDINATE ERROR. | |
|---|---|---|---|
| REMARK | 3 | ESU BASED ON R VALUE | (A): 0.129 |
| REMARK | 3 | ESU BASED ON FREE R VALUE | (A): 0.130 |
| REMARK | 3 | ESU BASED ON MAXIMUM LIKELIHOOD | (A): 0.123 |
| REMARK | 3 | ESU FOR B VALUES BASED ON MAXIMUM LIKELIHOOD | (A**2): 8.275 |
| REMARK | 3 | | |

| REMARK | 3 | CORRELATION COEFFICIENTS. | |
|---|---|---|---|
| REMARK | 3 | CORRELATION COEFFICIENT FO-FC | : 0.966 |
| REMARK | 3 | CORRELATION COEFFICIENT FO-FC FREE | : 0.946 |
| REMARK | 3 | | |

| REMARK | 3 | RMS DEVIATIONS FROM IDEAL VALUES | | COUNT | | RMS | | WEIGHT |
|---|---|---|---|---|---|---|---|---|
| REMARK | 3 | BOND LENGTHS REFINED ATOMS | (A): | 4331 | ; | 0.023 | ; | 0.022 |

| | | | | | | | | |
|---|---|---|---|---|---|---|---|---|
| REMARK | 3 | BOND ANGLES REFINED ATOMS | (DEGREES): | 5914 | ; | 2.066 | ; | 1.954 |
| REMARK | 3 | TORSION ANGLES, PERIOD 1 | DEGREES): | 558 | ; | 7.058 | ; | 5.000 |
| REMARK | 3 | TORSION ANGLES, PERIOD 2 | (DEGREES): | 183 | ; | 34.448 | ; | 24.098 |
| REMARK | 3 | TORSION ANGLES, PERIOD 3 | (DEGREES): | 719 | ; | 16.177 | ; | 15.000 |
| REMARK | 3 | TORSION ANGLES, PERIOD 4 | (DEGREES): | 22 | ; | 19.600 | ; | 15.000 |
| REMARK | 3 | CHIRAL-CENTER RESTRAINTS | (A**3): | 653 | ; | 0.150 | ; | 0.200 |

| | | | | | | | | |
|---|---|---|---|---|---|---|---|---|
| REMARK | 3 | GENERAL PLANES REFINED ATOMS | (A): | 3285 | ; | 0.011 | ; | 0.021 |
| REMARK | 3 | | | | | | | |

| | | | | | |
|---|---|---|---|---|---|
| REMARK | 3 | ISOTROPIC THERMAL FACTOR RESTRAINTS. | COUNT | RMS | WEIGHT |

| REMARK | 3 | | |
|---|---|---|---|
| REMARK | 3 | NCS RESTRAINTS STATISTICS | |
| REMARK | 3 | NUMBER OF NCS GROUPS : NULL | |
| REMARK | 3 | | |
| REMARK | 3 | TWIN DETAILS | |
| REMARK | 3 | NUMBER OF TWIN DOMAINS : NULL | |
| REMARK | 3 | | |
| REMARK | 3 | | |
| REMARK | 3 | TLS DETAILS | |
| REMARK | 3 | NUMBER OF TLS GROUPS : 3 | |
| REMARK | 3 | ATOM RECORD CONTAINS SUM OF TLS AND RESIDUAL B FACTORS | |
| REMARK | 3 | | |
| REMARK | 3 | TLS GROUP : 1 | |
| REMARK | 3 | NUMBER OF COMPONENTS GROUP : 2 | |
| REMARK | 3 | COMPONENTS C SSSEQI TO C SSSEQI | |
| REMARK | 3 | RESIDUE RANGE : L 1 L 107 | |
| REMARK | 3 | RESIDUE RANGE : H 1 H 126 | |
| REMARK | 3 | ORIGIN FOR THE GROUP (A): −4.5650 2.3930 61.1400 | |
| REMARK | 3 | T TENSOR | |
| REMARK | 3 | T11: 0.0609 T22: 0.1374 | |
| REMARK | 3 | T33: 0.1108 T12: 0.0152 | |
| REMARK | 3 | T13: 0.0371 T23: 0.0038 | |
| REMARK | 3 | L TENSOR | |
| REMARK | 3 | L11: 1.4342 L22: 1.9266 | |
| REMARK | 3 | L33: 1.5663 L12: 0.4534 | |
| REMARK | 3 | L13: 0.6824 L23: −0.0212 | |
| REMARK | 3 | S TENSOR | |
| REMARK | 3 | S11: −0.1037 S12: 0.0341 S13: 0.0803 | |
| REMARK | 3 | S21: 0.0178 S22: 0.0168 S23: −0.0443 | |
| REMARK | 3 | S31: −0.1403 S32: 0.0091 S33: 0.0868 | |
| REMARK | 3 | | |
| REMARK | 3 | TLS GROUP : 2 | |
| REMARK | 3 | NUMBER OF COMPONENTS GROUP : 2 | |
| REMARK | 3 | COMPONENTS C SSSEQI TO C SSSEQI | |
| REMARK | 3 | RESIDUE RANGE : L 108 L 250 | |
| REMARK | 3 | RESIDUE RANGE : H 127 H 250 | |
| REMARK | 3 | ORIGIN FOR THE GROUP (A): −9.4670 27.9380 37.8990 | |
| REMARK | 3 | T TENSOR | |
| REMARK | 3 | T11: 0.1828 T22: 0.1718 | |
| REMARK | 3 | T33: 0.1705 T12: 0.0152 | |
| REMARK | 3 | T13: −0.0018 T23: 0.0031 | |
| REMARK | 3 | L TENSOR | |
| REMARK | 3 | L11: 1.0826 L22: 2.6497 | |
| REMARK | 3 | L33: 1.7256 L12: 0.0323 | |
| REMARK | 3 | L13: 1.0191 L23: −0.0711 | |
| REMARK | 3 | S TENSOR | |
| REMARK | 3 | S11: 0.0788 S12: 0.1484 S13: −0.0374 | |
| REMARK | 3 | S21: −0.2102 S22: −0.0645 S23: −0.0600 | |
| REMARK | 3 | S31: 0.1386 S32: 0.1610 S33: −0.0143 | |
| REMARK | 3 | | |
| REMARK | 3 | TLS GROUP : 3 | |
| REMARK | 3 | NUMBER OF COMPONENTS GROUP : 1 | |
| REMARK | 3 | COMPONENTS C SSSEQI TO C SSSEQI | |
| REMARK | 3 | RESIDUE RANGE : I 1 I 200 | |
| REMARK | 3 | ORIGIN FOR THE GROUP (A): −4.6180 −12.7040 86.2350 | |
| REMARK | 3 | T TENSOR | |
| REMARK | 3 | T11: 0.2167 T22: 0.1761 | |
| REMARK | 3 | T33: 0.1149 T12: −0.0365 | |
| REMARK | 3 | T13: 0.0205 T23: −0.0179 | |
| REMARK | 3 | L TENSOR | |
| REMARK | 3 | L11: 2.1257 L22: 3.5572 | |
| REMARK | 3 | L33: 5.1547 L12: −0.4798 | |
| REMARK | 3 | L13: 0.9417 L23: −2.1956 | |
| REMARK | 3 | S TENSOR | |
| REMARK | 3 | S11: 0.0625 S12: −0.0117 S13: −0.0990 | |
| REMARK | 3 | S21: 0.1262 S22: −0.0921 S23: −0.0249 | |
| REMARK | 3 | S31: 0.1096 S32: 0.0337 S33: 0.0296 | |
| REMARK | 3 | | |
| REMARK | 3 | | |
| REMARK | 3 | BULK SOLVENT MODELLING. | |
| REMARK | 3 | METHOD USED : MASK | |
| REMARK | 3 | PARAMETERS FOR MASK CALCULATION | |
| REMARK | 3 | VDW PROBE RADIUS | : 1.20 |
| REMARK | 3 | ION PROBE RADIUS | : 0.80 |
| REMARK | 3 | SHRINKAGE RADIUS | : 0.80 |
| REMARK | 3 | | |

| | | |
|---|---|---|
| REMARK | 3 | OTHER REFINEMENT REMARKS: |
| REMARK | 3 | HYDROGENS HAVE BEEN USED IF PRESENT IN THE INPUT |
| REMARK | 3 | U VALUES : WITH TLS ADDED |
| REMARK | 3 | |
| SSBOND | 1 | CYS L 89 CYS L 23 |

| | | |
|---|---|---|
| LINKR | SG ACYS L 193 SG ACYS L 133 | SS |
| LINKR | SG BCYS L 193 SG BCYS L 133 | SS |

| | | |
|---|---|---|
| SSBOND | 2 | CYS H 96 CYS H 22 |
| SSBOND | 3 | CYS H 209 CYS H 153 |
| SSBOND | 4 | CYS I 71 CYS I 122 |

| | | |
|---|---|---|
| LINKR | SG ACYS I 78 SG ACYS I 125 | SS |
| LINKR | SG BCYS I 78 SG BCYS I 125 | SS |

| | | |
|---|---|---|
| CISPEP | 1 | SER L 7 PRO L 8 0.00 |
| CISPEP | 2 | TYR L 139 PRO L 140 0.00 |

| | | | |
|---|---|---|---|
| LINKR | SER H 140 | THR H 148 | gap |

| | | |
|---|---|---|
| CISPEP | 3 | PHE H 159 PRO H 160 0.00 |
| CISPEP | 4 | GLU H 161 PRO H 162 0.00 |

| | | | |
|---|---|---|---|
| LINKR | ASN I 88 | ASN I 92 | gap |
| LINKR | PRO I 108 | ARG I 119 | gap |
| LINKR | PHE I 60 | PRO I 64 | gap |

CRYST1 40.740 133.280 53.390 90.00 106.87 90.00 P 1 21 1
SCALE1 0.024546 0.000000 0.007445 0.00000
SCALE2 0.000000 0.007503 0.000000 0.00000
SCALE3 0.000000 0.000000 0.019573 0.00000

TABLE 2
hIL-21, chain I, (SEQ ID NO: 1) interactions with the the heavy
chain (chain H) of anti-IL-21 (SEQ ID NO: 17) and light chain
(chain L) of anti-IL-21 Fab (SEQ ID NO: 16). A distance cut-off
of 4.0 Å was used. The contacts were identified by the
CONTACT computer software program of the CCP4 suite [6].
In the last column “***” indicates a strong possibility
for a hydrogen bond at this contact (distance <3.3 Å)
as calculated by CONTACT, “*” indicates a weak possibility
(distance >3.3 Å). Blank indicates that the program considered
there to be no possibility of a hydrogen bond. Hydrogen-bonds
are specific between a donor and an acceptor, are typically strong,
and are easily identifiable.

| hIL-21 | | | aIL-21 | | | | |
|---|---|---|---|---|---|---|---|
| Res. | Res. # | Atom | Res. | Res. # | Atom | Distance | Possibly |
| Type | and Chain | name | Type | and Chain | name | [Å] | H-bond |
| Ile | 37 I | CG1 | Tyr | 109 H | CD1 | 3.50 | |
| | | | Tyr | 109 H | CE1 | 3.52 | |
| Ile | 37 I | CD1 | Tyr | 109 H | CD1 | 3.97 | |
| | | | Tyr | 109 H | CE1 | 3.79 | |
| Arg | 38 I | CB | Trp | 105 H | NE1 | 3.96 | |
| | | | Trp | 105 H | CE2 | 3.54 | |
| | | | Trp | 105 H | CH2 | 3.85 | |
| | | | Trp | 105 H | CZ2 | 3.48 | |
| Arg | 38 I | CG | Trp | 105 H | NE1 | 3.98 | |
| | | | Trp | 105 H | CE2 | 3.84 | |
| | | | Trp | 105 H | CD2 | 3.93 | |
| | | | Trp | 105 H | O | 3.26 | |
| Arg | 38 I | CD | Trp | 105 H | O | 3.64 | |
| Arg | 38 I | NE | Trp | 105 H | CA | 3.79 | |
| | | | Trp | 105 H | C | 3.62 | |
| | | | Trp | 105 H | O | 2.86 | *** |
| Arg | 38 I | CZ | Asp | 101 H | OD1 | 3.48 | |
| | | | Asp | 104 H | O | 3.78 | |
| | | | Asp | 108 H | O | 3.91 | |
| | | | Tyr | 109 H | CB | 3.45 | |
| | | | Trp | 105 H | O | 3.78 | |
| | | | Tyr | 109 H | CA | 3.88 | |
| Arg | 38 I | NH1 | Asp | 101 H | CG | 3.62 | |
| | | | Asp | 101 H | OD1 | 2.50 | *** |
| | | | Tyr | 109 H | CB | 3.26 | |
| | | | Tyr | 109 H | CA | 3.51 | |
| Arg | 38 I | NH2 | Asp | 101 H | OD1 | 3.64 | * |
| | | | Ser | 102 H | O | 3.30 | * |
| | | | Asp | 104 H | C | 3.82 | |
| | | | Asp | 104 H | O | 2.78 | *** |
| | | | Gly | 107 H | O | 3.70 | * |
| | | | Asp | 108 H | C | 3.81 | |
| | | | Asp | 108 H | O | 3.00 | *** |
| | | | Tyr | 109 H | CB | 3.80 | |
| | | | Trp | 105 H | O | 3.81 | * |
| | | | Tyr | 109 H | CA | 3.89 | |
| Gln | 41 I | CG | Phe | 111 H | CZ | 3.63 | |
| Gln | 41 I | CD | Phe | 111 H | CE1 | 3.73 | |
| | | | Phe | 111 H | CZ | 3.83 | |
| Gln | 41 I | NE2 | Phe | 111 H | CD1 | 3.91 | |
| | | | Phe | 111 H | CE1 | 3.44 | |
| | | | Phe | 111 H | CZ | 3.83 | |
| | | | Tyr | 109 H | O | 2.92 | *** |
| Asp | 44 I | CA | Ser | 30 L | OG | 3.61 | |
| Asp | 44 I | CB | Ser | 32 L | CB | 3.48 | |
| | | | Ser | 30 L | OG | 3.49 | |
| | | | Ser | 32 L | OG | 3.47 | |
| Asp | 44 I | CG | Ser | 32 L | CB | 3.69 | |
| | | | Ser | 30 L | OG | 3.79 | |
| | | | Ser | 32 L | OG | 3.41 | |
| Asp | 44 I | OD2 | Ser | 32 L | N | 3.30 | *** |
| | | | Ser | 32 L | CA | 3.60 | |
| | | | Ser | 32 L | CB | 2.98 | |
| | | | Ser | 32 L | OG | 2.62 | *** |
| Asp | 44 I | C | Ser | 30 L | OG | 3.70 | |
| Asp | 44 I | O | Tyr | 33 L | CE2 | 3.80 | |
| | | | Ser | 30 L | CB | 3.57 | |
| | | | Ser | 30 L | OG | 3.07 | *** |
| Ile | 45 I | CA | Tyr | 33 L | OH | 3.53 | |
| Ile | 45 I | CB | Tyr | 33 L | OH | 3.72 | |
| Ile | 45 I | CG1 | Tyr | 33 L | OH | 3.62 | |
| Ile | 45 I | CD1 | Phe | 111 H | CZ | 3.92 | |
| Ile | 45 I | CG2 | Tyr | 33 L | OH | 3.61 | |
| Asp | 47 I | CB | Ser | 30 L | OG | 3.95 | |
| Gln | 48 I | CB | Tyr | 33 L | CZ | 3.66 | |
| | | | Tyr | 33 L | OH | 3.82 | |
| | | | Tyr | 33 L | CE2 | 3.73 | |
| | | | Ser | 30 L | CB | 3.94 | |
| Gln | 48 I | CG | Tyr | 33 L | CZ | 3.75 | |
| | | | Tyr | 33 L | CD1 | 3.93 | |
| | | | Tyr | 33 L | CE1 | 3.68 | |
| Gln | 48 I | CD | Gln | 91 L | NE2 | 3.64 | |
| | | | Ser | 30 L | N | 3.75 | |
| Gln | 48 I | OE1 | Tyr | 33 L | CD1 | 3.82 | |
| | | | Gln | 91 L | NE2 | 3.35 | * |
| | | | Tyr | 33 L | CB | 3.84 | |
| | | | Tyr | 33 L | CG | 3.66 | |
| | | | Tyr | 33 L | CD2 | 3.99 | |
| | | | Ser | 30 L | N | 2.90 | *** |
| | | | Ser | 30 L | CA | 3.83 | |
| | | | Ser | 30 L | CB | 3.56 | |
| | | | Val | 29 L | CG1 | 3.92 | |
| | | | Val | 29 L | CA | 3.55 | |
| | | | Val | 29 L | C | 3.69 | |
| Gln | 48 I | NE2 | Gln | 91 L | NE2 | 3.73 | * |
| | | | Ser | 28 L | O | 3.30 | *** |
| Asn | 51 I | CG | Ser | 28 L | O | 3.97 | |
| Asn | 51 I | OD1 | Ser | 28 L | CB | 3.80 | |
| Asn | 51 I | ND2 | Ser | 28 L | O | 3.52 | * |
| Tyr | 52 I | CE1 | Gln | 27 L | OE1 | 3.42 | |
| Tyr | 52 I | CZ | Gln | 27 L | OE1 | 3.54 | |
| Tyr | 52 I | OH | Gln | 27 L | CD | 3.85 | |
| | | | Gln | 27 L | OE1 | 2.69 | *** |
| Asn | 92 I | OD1 | Glu | 1 L | N | 3.15 | *** |
| | | | Gln | 27 L | OE1 | 3.89 | * |
| Arg | 94 I | CG | Tyr | 59 H | CD1 | 3.85 | |
| | | | Gly | 93 L | O | 3.43 | |
| Arg | 94 I | CD | Tyr | 59 H | CB | 3.83 | |
| | | | Trp | 47 H | CZ3 | 3.85 | |
| | | | Trp | 47 H | CH2 | 3.57 | |
| | | | Gly | 93 L | O | 3.72 | |
| Arg | 94 I | NE | Trp | 47 H | CZ3 | 3.52 | |
| | | | Trp | 47 H | CH2 | 3.79 | |
| | | | Gly | 93 L | O | 3.64 | * |
| | | | Ser | 94 L | C | 3.90 | |
| | | | Ser | 94 L | O | 3.27 | *** |
| Arg | 94 I | CZ | Trp | 47 H | CZ3 | 3.39 | |

### Example 2

### Crystal Structure of hIL-21 in Complex with the Extracellular Domain of hIL-21Rα

The 3-dimensional structure of hIL-21 in complex with a soluble fragment of hIL-21Rα was solved and refined to 2.8 Å resolution using X-ray crystallography. The results demonstrate a binding of hIL-21Rα to helix A and C of hIL-21. Thus, the hIL-21Rα binding site on hIL-21 is very similar to that described for the binding of anti-IL-21 to hIL-21 in Example 1, proving a direct binding competition for the two molecules to hIL-21.

### Materials and Methods

### Complex Formation

The Complex of hIL-21 (residues 30-162 of SEQ ID NO:1) and hIL-21Rα (SEQ ID NO:14) was formed by mixing hIL-21 and hIL-21Rα at room temprature at a ratio of 1.5:1. Excess levels of hIL-21 were used as hIL-21 is most readily available. Furthermore, as hIL-21 (≈15-16 kDa) is much smaller than hIL-21Rα (28 kDa) it is more easily separated from the complex (≈43 kDA) by gelfiltration. The complex was loaded on to a HiLoad 16/60 Superdex 75 column (GFC) (GE Healthcare) and eluted with PBS buffer (10 mM phosphate, 150 mM NaCl, pH 7.4). The fractions containing the complex were concentrated to 5 mg/ml using an Amicon Ultra-4 centrifugal filter device with a 10000 Da molecular weight cut-off. The following additional mutations were, however, introduced in the mature IL-21R in order to facilitate the crystallization process: N80Q, N87Q, N118Q, and N₁₀₈D.

### Crystallization

All crystals were grown at 18° C. in sitting drops with a reservoir solution containing 100 μL of 1.8-1.9 M di-ammonium sulphate and 0.1 M sodium acetate at ph 5.5. 1 μL of reservoir solution and 1 μL protein solution was mixed in the pedestal. Large single crystals appeared after 10-14 days. These were flash frozen using a cryo solution containing 3.0 M di-ammonium sulphate and 0.1 M sodium acetate at ph 5.5.

Tantalum bromide derivatives were obtained by adding 0.1 μL of a 2 mM Ta₆Br₂ solution. This was left for 2 hours at which point the crystals had turned green. The crystals were flash frozen using a cryo solution containing 3.0 M di-ammonium sulphate and 0.1 M sodium acetate at ph 5.5.

Crystals of seleno-metheonine hIL-21 (semen-21) in complex with hIL-21Rα were produced in the same way as wild type protein.

### Data Collection

All data were collected at the Swiss Light Source (SLS) at the PXIII (X06DA) beamline. For the native dataset 180 frames were collected with an oscillation of 1.0 degree. For the Se-metheonine dataset 720 frames were collected with an oscillation of 1.0 degree and for the tantalum bromide dataset 1080 frames were collected with an oscillation of 1.0 degree. Space group determination, integration and scaling of the data were made by the xds software package [2]. Cell parameters for the data were determined to be 83, 152, 365 Å, for a, b and c, respectively, and the space group P2₁2₁2₁, with 8 independent hIL-21/hIL-21Rα complex molecules per asymmetric unit.

### Phasing, Model Building and Refinement

The electron density map generated using the phases from the tantalum bromide and Se-metheonine datasets were used for building an initial model. Using the Se-metheonine sites 8 copies of the known NMR structure of hIL-21 [14] could be placed in the map. From this model initial non-crystallographic symmetry (NCS) operators were calculated and new phases were derived by making use of NCS-restraints. The new phases were used along with the experimental phases to calculate an improved electron density map. A poly-alanine model based on IL-2N3 coordinates was created and divided into its two constituent fibronectin domains. The two fibronectin domains were placed in the regions of the map containing density for hIL-21Rα. Using the Resolve software [15], one NCS group with 8 operators was made for hIL-21 as well as for each of the two fibronectin domains of IL-2Rβ/IL-21Rα. This was followed by density modification and phase extension in Resolve. The hIL-21Rα structure was built in the molecular graphics software Coot [13], crystallographic refinements by the software program Phenix.refine [11] of the Phenix software package [12] was used and was followed by phase improvements by Resolve, respectively. The procedure was cycled until no further significant improvements could be made to the model. The final model, containing 8 molecules of both hIL-21Rα and hIL-21, forming the hIL-21Rα/hIL-21 complex, was refined to 2.8 Å resolution. Final R- and R-free for data with F(obs)/σ[F(obs)] larger than 2.0 were 0.246 and 0.274, respectively, and the model showed a root-mean-square deviation (RMSD) from ideal bond lengths of 0.011 Å (Table 3).

### Results

The crystal structure of hIL-21 in complex with a soluble fragment of hIL-21Rα demonstrated a binding of hIL-21Rα to helix A and C of hIL-21. Thus, the hIL-21Rα binding site on hIL-21 is very similar to that described for the binding of aIL-21 in Example 1 proving a direct binding competition for the two molecules to hIL-21.

Calculation of the areas excluded in pair-wise interactions by the software program AREAIMOL of the CCP4 program suite [6] gave for the hIL-21/hIL-21Rα molecular complex of the crystal structure 984 for hIL-21 and 998 Å² for hIL-21Rα, respectively. The average area excluded in pair-wise interaction between the hIL-21 and hIL-21Rα molecules was calculated to be 991 Å².

The direct contacts between the hIL-21 and hIL-21Rα were identified by running the CONTACT software of the CCP4 program suite [6] using a cut-off distance of 4.0 Å between the hIL-21Rα and the hIL-21 molecules. The results from one of the hIL-21/hIL-21Rα complexes, out of the eight NCS-restrained complexes contained in the asymmetric unit, are shown in Table 4. The resulting hIL-21 interface residues for hIL-21Rα was found to comprise the following residues of hIL-21 (SEQ ID NO: 1, Table 4): His 35, Ile 37, Arg 38, Arg 40, Gln 41, Asp 44, Ile 45, Gln 48, Tyr 52, Ile 95, Val 98, Ser 99, Lys 102, Arg 105, Lys 106, Pro 107, Pro 108 and Ser 109.

Thus, the interface residues hIL-21 for hIL-21Rα comprises residues of helix A and C. These contact areas agreed very well with what have been determined as the binding site for anti-IL-21 on hIL-21 (Example 1).

The hIL-21Rα interface residues for hIL-21 included residues Tyr 12, Gln 35, Gln 37, Tyr 38, Glu 40, Leu 41, Phe 69, His 70, Phe 71, Met 72, Ala 73, Asp 74, Asp 75, Ile 76, Leu 96, Ala 98, Pro 128, Ala 129, Tyr 131, Met 132, Lys 136, Ser 192 and Tyr 193 of the hIL-21Rα chain (SEQ ID NO: 14, Table 4).

TABLE 3
Results from the X-ray model refinement to the observed data of the hiL-21/hIL-21Rα
complex by the software program phenix.refine [5] of the phenix software package [12].

| | |
|---|---|
| REMARK | PHENIX refinement |
| REMARK | ******************** REFINEMENT SUMMARY: QUICK FACTS ******************** |
| REMARK | Start: r_work = 0.2539 r_free = 0.2793 bonds = 0.019 angles = 1.848 |
| REMARK | Final: r_work = 0.2457 r_free = 0.2744 bonds = 0.011 angles = 1.504 |
| REMARK | ******************************************************************************* |
| REMARK | |
| REMARK | ****************** REFINEMENT STATISTICS STEP BY STEP ********************** |
| REMARK | leading digit, like 1_, means number of macro-cycle |
| REMARK | 0: statistics at the very beginning when nothing is done yet |
| REMARK | 1_bss: bulk solvent correction and/or (anisotropic) scaling |
| REMARK | 1_xyz: refinement of coordinates |
| REMARK | 1_adp: refinement of ADPs (Atomic Displacement Parameters) |
| REMARK | 1_wat: ordered solvent update (add/remove) |
| REMARK | 1_occ: refinement of occupancies |
| REMARK | ------------------------------------------------------------------------------------------------------------------------ |
| REMARK | R-factors, x-ray target values and norm of gradient of x-ray target |

| | | | | | |
|---|---|---|---|---|---|
| REMARK | stage | r-work | r-free | xray_target_w | xray_target_t |
| REMARK | 0: | 0.4488 | 0.4688 | 9.317485e−01 | 9.748372e−01 |
| REMARK | 1_bss: | 0.2539 | 0.2793 | 1.051547e+00 | 1.105637e+00 |
| REMARK | 1_xyz: | 0.2484 | 0.2800 | 1.039589e+00 | 1.110778e+00 |
| REMARK | 1_adp: | 0.2452 | 0.2756 | 1.056595e+00 | 1.128706e+00 |
| REMARK | 2_bss: | 0.2446 | 0.2742 | 1.063843e+00 | 1.133861e+00 |
| REMARK | 2_xyz: | 0.2475 | 0.2756 | 1.064345e+00 | 1.128312e+00 |
| REMARK | 2_adp: | 0.2459 | 0.2752 | 1.063428e+00 | 1.130410e+00 |
| REMARK | 3_bss: | 0.2457 | 0.2739 | 1.065109e+00 | 1.131133e+00 |
| REMARK | 3_xyz: | 0.2456 | 0.2744 | 1.062540e+00 | 1.129894e+00 |
| REMARK | 3_adp: | 0.2453 | 0.2745 | 1.062171e+00 | 1.130175e+00 |
| REMARK | 3_bss: | 0.2457 | 0.2744 | 1.064393e+00 | 1.131138e+00 |

| REMARK | ------------------------------------------------------------------------------------------------------------------------ | | | | | | | | |
|---|---|---|---|---|---|---|---|---|---|
| REMARK | stage | k_sol | b_sol | b11 | b22 | b33 | b12 | b13 | b23 |
| REMARK | 0: | 0.000 | 0.000 | 0.000 | 0.000 | 0.000 | 0.000 | 0.000 | 0.000 |
| REMARK | 1_bss: | 0.331 | 37.216 | 4.485 | 1.765 | −6.250 | 0.000 | −0.000 | −0.000 |
| REMARK | 1_xyz: | 0.331 | 37.216 | 4.485 | 1.765 | −6.250 | 0.000 | −0.000 | −0.000 |
| REMARK | 1_adp: | 0.331 | 37.216 | 4.485 | 1.765 | −6.250 | 0.000 | −0.000 | −0.000 |
| REMARK | 2_bss: | 0.331 | 37.216 | 4.485 | 1.765 | −6.250 | 0.000 | −0.000 | −0.000 |
| REMARK | 2_xyz: | 0.331 | 37.216 | 4.485 | 1.765 | −6.250 | 0.000 | −0.000 | −0.000 |
| REMARK | 2_adp: | 0.331 | 37.216 | 4.485 | 1.765 | −6.250 | 0.000 | −0.000 | −0.000 |
| REMARK | 3_bss: | 0.331 | 39.746 | 4.555 | 1.712 | −6.267 | 0.000 | −0.000 | −0.000 |
| REMARK | 3_xyz: | 0.331 | 39.746 | 4.555 | 1.712 | −6.267 | 0.000 | −0.000 | −0.000 |
| REMARK | 3_adp: | 0.331 | 39.746 | 4.555 | 1.712 | −6.267 | 0.000 | −0.000 | −0.000 |
| REMARK | 3_bss: | 0.331 | 39.746 | 4.555 | 1.712 | −6.267 | 0.000 | −0.000 | −0.000 |

| REMARK | ------------------------------------------------------------------------------------------------------------------------ | | | | |
|---|---|---|---|---|---|
| REMARK | stage | <pher> | fom | alpha | beta |
| REMARK | 0: | 33.262 | 0.7289 | 1.1099 | 315581.540 |
| REMARK | 1_bss: | 29.401 | 0.7713 | 1.1878 | 165072.087 |
| REMARK | 1_xyz: | 29.346 | 0.7717 | 1.1909 | 164156.763 |
| REMARK | 1_adp: | 28.860 | 0.7771 | 1.1825 | 159413.412 |
| REMARK | 2_bss: | 28.721 | 0.7786 | 1.1844 | 157394.660 |
| REMARK | 2_xyz: | 28.854 | 0.7773 | 1.1833 | 159060.536 |
| REMARK | 2_adp: | 28.774 | 0.7781 | 1.1749 | 158532.819 |
| REMARK | 3_bss: | 28.762 | 0.7782 | 1.2308 | 157355.227 |
| REMARK | 3_xyz: | 28.822 | 0.7775 | 1.2298 | 157658.005 |
| REMARK | 3_adp: | 28.811 | 0.7776 | 1.2200 | 157604.175 |
| REMARK | 3_bss: | 28.803 | 0.7777 | 1.2198 | 157356.836 |

| REMARK | ------------------------------------------------------------------------------------------------------------------------ | | | | | | | |
|---|---|---|---|---|---|---|---|---|
| REMARK | stage | angl | bond | chir | dihe | plan | repu | geom_target |
| REMARK | 0: | 1.848 | 0.019 | 0.238 | 26.001 | 0.005 | 4.112 | 2.9259e−01 |
| REMARK | 1_bss: | 1.848 | 0.019 | 0.238 | 26.001 | 0.005 | 4.112 | 2.9259e−01 |
| REMARK | 1_xyz: | 1.771 | 0.016 | 0.117 | 26.061 | 0.006 | 4.114 | 2.2556e−01 |
| REMARK | 1_adp: | 1.771 | 0.016 | 0.117 | 26.061 | 0.006 | 4.114 | 2.2556e−01 |
| REMARK | 2_bss: | 1.771 | 0.016 | 0.117 | 26.061 | 0.006 | 4.114 | 2.2556e−01 |
| REMARK | 2_xyz: | 1.533 | 0.011 | 0.102 | 25.835 | 0.005 | 4.116 | 1.7700e−01 |
| REMARK | 2_adp: | 1.533 | 0.011 | 0.102 | 25.835 | 0.005 | 4.116 | 1.7700e−01 |
| REMARK | 3_bss: | 1.533 | 0.011 | 0.102 | 25.835 | 0.005 | 4.116 | 1.7700e−01 |
| REMARK | 3_xyz: | 1.504 | 0.011 | 0.100 | 25.623 | 0.005 | 4.117 | 1.7188e−01 |
| REMARK | 3_adp: | 1.504 | 0.011 | 0.100 | 25.623 | 0.005 | 4.117 | 1.7188e−01 |
| REMARK | 3_bss: | 1.504 | 0.011 | 0.100 | 25.623 | 0.005 | 4.116 | 1.7189e−01 |
| REMARK | ------------------------------------------------------------------------------------------------------------------------ | | | | | | | |
| REMARK | Maximal deviations: | | | | | | | |
| REMARK | stage | angl | bond | chir | dihe | plan | repu | \|grad\| |
| REMARK | 0: | 54.912 | 1.371 | 4.78617 | 9.132 | 0.063 | 0.497 | 1.0293e−01 |
| REMARK | 1_bss: | 54.912 | 1.371 | 4.78617 | 9.132 | 0.063 | 0.497 | 1.0293e−01 |
| REMARK | 1_xyz: | 38.380 | 0.259 | 1.30417 | 9.999 | 0.066 | 1.536 | 4.7297e−02 |
| REMARK | 1_adp: | 38.380 | 0.259 | 1.30417 | 9.999 | 0.066 | 1.536 | 4.7297e−02 |
| REMARK | 2_bss: | 38.380 | 0.259 | 1.30417 | 9.999 | 0.066 | 1.536 | 4.7297e−02 |
| REMARK | 2_xyz: | 37.537 | 0.246 | 1.18317 | 8.026 | 0.050 | 1.614 | 3.3151e−02 |
| REMARK | 2_adp: | 37.537 | 0.246 | 1.18317 | 8.026 | 0.050 | 1.614 | 3.3151e−02 |
| REMARK | 3_bss: | 37.537 | 0.246 | 1.18317 | 8.026 | 0.050 | 1.614 | 3.3151e−02 |
| REMARK | 3_xyz: | 36.658 | 0.227 | 1.19117 | 8.542 | 0.050 | 1.616 | 3.0834e−02 |
| REMARK | 3_adp: | 36.658 | 0.227 | 1.19117 | 8.542 | 0.050 | 1.616 | 3.0834e−02 |
| REMARK | 3_bss: | 36.658 | 0.227 | 1.19117 | 8.542 | 0.050 | 1.616 | 3.0835e−02 |

| REMARK | ------------------------------------------------------------------------------------------------------------------------ | | | |
|---|---|---|---|---|
| REMARK | stage | b_max | b_min | b_ave |
| REMARK | 0: | 176.45 | 16.23 | 64.56 |
| REMARK | 1_bss: | 176.62 | 16.40 | 64.73 |
| REMARK | 1_xyz: | 176.62 | 16.40 | 64.73 |
| REMARK | 1_adp: | 170.81 | 17.21 | 63.95 |
| REMARK | 2_bss: | 170.81 | 17.21 | 63.95 |
| REMARK | 2_xyz: | 170.81 | 17.21 | 63.95 |
| REMARK | 2_adp: | 169.63 | 15.75 | 63.56 |
| REMARK | 3_bss: | 172.16 | 18.28 | 66.09 |
| REMARK | 3_xyz: | 172.16 | 18.28 | 66.09 |
| REMARK | 3_adp: | 172.29 | 16.84 | 65.68 |
| REMARK | 3_bss: | 172.29 | 16.84 | 65.68 |
| REMARK | ------------------------------------------------------------------------------------------------------------------------ | | | |
| REMARK | stage | Deviation of refined | | |
| REMARK | | model from start model | | |
| REMARK | | max | min | mean |
| REMARK | 0: | 0.000 | 0.000 | 0.000 |
| REMARK | 1_bss: | 0.000 | 0.000 | 0.000 |
| REMARK | 1_xyz: | 6.679 | 0.001 | 0.065 |
| REMARK | 1_adp: | 6.679 | 0.001 | 0.065 |
| REMARK | 2_bss: | 6.679 | 0.001 | 0.065 |
| REMARK | 2_xyz: | 6.863 | 0.000 | 0.072 |
| REMARK | 2_adp: | 6.863 | 0.000 | 0.072 |
| REMARK | 3_bss: | 6.863 | 0.000 | 0.072 |
| REMARK | 3_xyz: | 7.259 | 0.001 | 0.087 |
| REMARK | 3_adp: | 7.259 | 0.001 | 0.087 |
| REMARK | 3_bss: | 7.259 | 0.001 | 0.087 |

| REMARK | ------------------------------------------------------------------------------------------------------------------------ | | |
|---|---|---|---|
| REMARK | MODEL CONTENT. | | |
| REMARK | ELEMENT | ATOM RECORD COUNT | OCCUPANCY SUM |
| REMARK | Ni | 8 | 8.00 |
| REMARK | C | 13898 | 13898.00 |
| REMARK | S | 160 | 160.00 |
| REMARK | O | 4446 | 4446.00 |
| REMARK | N | 3672 | 3672.00 |
| REMARK | TOTAL | 22184 | 22184.00 |

| REMARK | ------------------------------------------------------------------------------------------------------------------------ | | |
|---|---|---|---|
| REMARK | r_free_flags.md5.hexdigest ddcaa2b4f6cee21eff5cf80a2a17d630 | | |
| REMARK | | | |
| REMARK | IF THIS FILE IS FOR PDB DEPOSITION: REMOVE ALL FROM THIS LINE UP. | | |
| REMARK | 3 | | |
| REMARK | 3 | REFINEMENT. | |
| REMARK | 3 | PROGRAM | : PHENIX (phenix.refine: 1.6.4_486) |
| REMARK | 3 | AUTHORS | : Adams, Afonine, Chen, Davis, Echols, Gopal, |
| REMARK | 3 | | : Grosse-Kunstleve, Headd, Hung, Immormino, Ioerger, McCoy, |
| REMARK | 3 | | : McKee, Moriarty, Pai, Read, Richardson, Richardson, Romo, |
| REMARK | 3 | | : Sacchettini, Sauter, Smith, Storoni, Terwilliger, Zwart |

| REMARK | 3 | | |
|---|---|---|---|
| REMARK | 3 | REFINEMENT TARGET: MLHL | |
| REMARK | 3 | | |
| REMARK | 3 | DATA USED IN REFINEMENT. | |
| REMARK | 3 | RESOLUTION RANGE HIGH (ANGSTROMS) | : 2.800 |
| REMARK | 3 | RESOLUTION RANGE LOW (ANGSTROMS) | : 80.218 |
| REMARK | 3 | MIN(FOBS/SIGMA_FOBS) | : 1.99 |
| REMARK | 3 | COMPLETENESS FOR RANGE (%) | : 99.67 |
| REMARK | 3 | NUMBER OF REFLECTIONS | : 112598 |
| REMARK | 3 | | |
| REMARK | 3 | FIT TO DATA USED IN REFINEMENT. | |
| REMARK | 3 | R VALUE (WORKING + TEST SET) | : 0.2471 |
| REMARK | 3 | R VALUE (WORKING SET) | : 0.2457 |
| REMARK | 3 | FREE R VALUE | : 0.2744 |
| REMARK | 3 | FREE R VALUE TEST SET SIZE (%) | : 5.00 |
| REMARK | 3 | FREE R VALUE TEST SET COUNT | : 5632 |
| REMARK | 3 | | |

| REMARK | 3 | FIT TO DATA USED IN REFINEMENT (IN BINS). | | | | | | |
|---|---|---|---|---|---|---|---|---|
| REMARK | 3 | BIN | RESOLUTION RANGE | COMPL. | NWORK | NFREE | RWORK | RFREE |
| REMARK | 3 | 1 | 80.2504-8.6967 | 0.99 | 3841 | 204 | 0.2304 | 0.2670 |
| REMARK | 3 | 2 | 8.6967-6.9040 | 1.00 | 3676 | 194 | 0.1930 | 0.2079 |
| REMARK | 3 | 3 | 6.9040-6.0316 | 1.00 | 3662 | 193 | 0.2120 | 0.2524 |
| REMARK | 3 | 4 | 6.0316-5.4803 | 1.00 | 3614 | 190 | 0.2212 | 0.2323 |
| REMARK | 3 | 5 | 5.4803-5.0876 | 1.00 | 3626 | 191 | 0.2213 | 0.2531 |
| REMARK | 3 | 6 | 5.0876-4.7877 | 1.00 | 3613 | 190 | 0.2116 | 0.2365 |
| REMARK | 3 | 7 | 4.7877-4.5479 | 1.00 | 3567 | 188 | 0.2000 | 0.2206 |
| REMARK | 3 | 8 | 4.5479-4.3500 | 1.00 | 3580 | 188 | 0.1998 | 0.2308 |
| REMARK | 3 | 9 | 4.3500-4.1825 | 1.00 | 3588 | 189 | 0.2053 | 0.2077 |
| REMARK | 3 | 10 | 4.1825-4.0382 | 1.00 | 3594 | 189 | 0.2107 | 0.2477 |
| REMARK | 3 | 11 | 4.0382-3.9119 | 1.00 | 3514 | 185 | 0.2353 | 0.2636 |
| REMARK | 3 | 12 | 3.9119-3.8001 | 1.00 | 3576 | 188 | 0.2506 | 0.2807 |
| REMARK | 3 | 13 | 3.8001-3.7001 | 1.00 | 3548 | 187 | 0.2468 | 0.2855 |
| REMARK | 3 | 14 | 3.7001-3.6098 | 1.00 | 3540 | 186 | 0.2398 | 0.2698 |
| REMARK | 3 | 15 | 3.6098-3.5277 | 1.00 | 3550 | 187 | 0.2523 | 0.2707 |
| REMARK | 3 | 16 | 3.5277-3.4527 | 1.00 | 3521 | 185 | 0.2572 | 0.2751 |
| REMARK | 3 | 17 | 3.4527-3.3836 | 1.00 | 3571 | 188 | 0.2651 | 0.2886 |
| REMARK | 3 | 18 | 3.3836-3.3197 | 1.00 | 3529 | 186 | 0.2702 | 0.2857 |
| REMARK | 3 | 19 | 3.3197-3.2604 | 1.00 | 3526 | 185 | 0.2773 | 0.3256 |
| REMARK | 3 | 20 | 3.2604-3.2052 | 1.00 | 3543 | 187 | 0.2955 | 0.3397 |
| REMARK | 3 | 21 | 3.2052-3.1535 | 1.00 | 3491 | 183 | 0.3001 | 0.3201 |
| REMARK | 3 | 22 | 3.1535-3.1050 | 1.00 | 3574 | 188 | 0.3028 | 0.3841 |
| REMARK | 3 | 23 | 3.1050-3.0593 | 1.00 | 3490 | 184 | 0.3241 | 0.3676 |
| REMARK | 3 | 24 | 3.0593-3.0162 | 1.00 | 3518 | 185 | 0.3187 | 0.3739 |
| REMARK | 3 | 25 | 3.0162-2.9754 | 1.00 | 3567 | 188 | 0.3274 | 0.3640 |
| REMARK | 3 | 26 | 2.9754-2.9368 | 1.00 | 3479 | 183 | 0.3501 | 0.3890 |
| REMARK | 3 | 27 | 2.9368-2.9001 | 0.99 | 3526 | 186 | 0.3454 | 0.3774 |
| REMARK | 3 | 28 | 2.9001-2.8651 | 1.00 | 3508 | 184 | 0.3485 | 0.3573 |
| REMARK | 3 | 29 | 2.8651-2.8318 | 0.99 | 3504 | 185 | 0.3523 | 0.3921 |
| REMARK | 3 | 30 | 2.8318-2.8000 | 0.99 | 3530 | 186 | 0.3696 | 0.4033 |
| REMARK | 3 | | | | | | | |

| REMARK | 3 | BULK SOLVENT MODELLING. | |
|---|---|---|---|
| REMARK | 3 | METHOD USED | : FLAT BULK SOLVENT MODEL |
| REMARK | 3 | SOLVENT RADIUS | : 1.00 |
| REMARK | 3 | SHRINKAGE RADIUS | : 0.72 |
| REMARK | 3 | GRID STEP FACTOR | : 4.00 |
| REMARK | 3 | K_SOL | : 0.331 |
| REMARK | 3 | B_SOL | : 39.746 |

| REMARK | 3 | | | | |
|---|---|---|---|---|---|
| REMARK | 3 | ERROR ESTIMATES. | | | |
| REMARK | 3 | COORDINATE ERROR (MAXIMUM-LIKELIHOOD BASED): 0.39 | | | |
| REMARK | 3 | PHASE ERROR (DEGREES, MAXIMUM-LIKELIHOOD BASED): 28.80 | | | |
| REMARK | 3 | | | | |
| REMARK | 3 | OVERALL SCALE FACTORS. | | | |
| REMARK | 3 | SCALE = SUM(\|F_OBS\|*\|F_MODEL\|)/SUM(\|F_MODEL\|**2): 1.3488 | | | |
| REMARK | 3 | ANISOTROPIC SCALE MATRIX ELEMENTS (IN CARTESIAN BASIS). | | | |
| REMARK | 3 | B11: 4.5554 | | | |
| REMARK | 3 | B22: 1.7115 | | | |
| REMARK | 3 | B33: −6.2669 | | | |
| REMARK | 3 | B12: 0.0000 | | | |
| REMARK | 3 | B13: −0.0000 | | | |
| REMARK | 3 | B23: −0.0000 | | | |
| REMARK | 3 | | | | |
| REMARK | 3 | R FACTOR FORMULA. | | | |
| REMARK | 3 | R = SUM(\|\|F_OBS\|−SCALE*\|F_MODEL\|\|)/SUM(\|F_OBS\|) | | | |
| REMARK | 3 | | | | |
| REMARK | 3 | TOTAL MODEL STRUCTURE FACTOR (F_MODEL). | | | |
| REMARK | 3 | F_MODEL = FB_CART * (F_CALC_ATOMS + F_BULK) | | | |
| REMARK | 3 | F_BULK = K_SOL * EXP(−B_SOL * S**2/4) * F_MASK | | | |
| REMARK | 3 | F_CALC_ATOMS = ATOMIC MODEL STRUCTURE FACTORS | | | |
| REMARK | 3 | FB_CART = EXP(−H(t) * A(−1) * B * A(−1t) * H) | | | |
| REMARK | 3 | A = orthogonalization matrix, H = MILLER INDEX | | | |
| REMARK | 3 | (t) = TRANSPOSE, (−1) = INVERSE | | | |
| REMARK | 3 | | | | |
| REMARK | 3 | STRUCTURE FACTORS CALCULATION ALGORITHM: FFT | | | |
| REMARK | 3 | | | | |
| REMARK | 3 | DEVIATIONS FROM IDEAL VALUES. | | | |
| REMARK | 3 | | RMSD | MAX | COUNT |
| REMARK | 3 | BOND: | 0.011 | 0.227 | 22827 |
| REMARK | 3 | ANGLE: | 1.504 | 36.658 | 31021 |
| REMARK | 3 | CHIRALITY: | 0.100 | 1.191 | 3480 |
| REMARK | 3 | PLANARITY: | 0.005 | 0.050 | 3877 |
| REMARK | 3 | DIHEDRAL: | 25.623 | 178.542 | 8835 |

| REMARK | 3 | MIN NONBONDED DISTANCE: 1.616 | | |
|---|---|---|---|---|
| REMARK | 3 | | | |
| REMARK | 3 | ATOMIC DISPLACEMENT PARAMETERS. | | |
| REMARK | 3 | WILSON B: None | | |
| REMARK | 3 | RMS(B_ISO_OR_EQUIVALENT_BONDED): 8.91 | | |
| REMARK | 3 | ATOMS | NUMBER OF ATOMS | |
| REMARK | 3 | | ISO. | ANISO. |
| REMARK | 3 | ALL: | 22184 | 0 |
| REMARK | 3 | ALL (NO H): | 22184 | 0 |
| REMARK | 3 | SOLVENT: | 0 | 0 |
| REMARK | 3 | NON-SOLVENT: | 22184 | 0 |
| REMARK | 3 | HYDROGENS: | 0 | 0 |

| | | | |
|---|---|---|---|
| REMARK | 3 | | |
| REMARK | 3 | NCS DETAILS. | |
| REMARK | 3 | NUMBER OF NCS GROUPS | : 2 |
| REMARK | 3 | NCS GROUP: 1 | |
| REMARK | 3 | NCS OPERATOR: 1 | |
| REMARK | 3 | REFERENCE SELECTION | : chain A and (resseq 3:159 or |
| REMARK | 3 | | : resseq 164:209 or resseq 569:575) |
| REMARK | 3 | SELECTION: | : chain C and (resseq 3:159 or |
| REMARK | 3 | | : resseq 164:209 or resseq 569:575) |
| REMARK | 3 | ATOM PAIRS NUMBER | : 1737 |
| REMARK | 3 | RMSD | : 0.058 |
| REMARK | 3 | NCS OPERATOR: 2 | |
| REMARK | 3 | REFERENCE SELECTION | : chain A and (resseq 3:159 or |
| REMARK | 3 | | : resseq 164:209 or resseq 569:575) |
| REMARK | 3 | SELECTION | : chain E and (resseq 3:159 or |
| REMARK | 3 | | : resseq 164:209 or resseq 569:575) |
| REMARK | 3 | ATOM PAIRS NUMBER | : 1737 |
| REMARK | 3 | RMSD | : 0.058 |
| REMARK | 3 | NCS OPERATOR: 3 | |
| REMARK | 3 | REFERENCE SELECTION | : chain A and (resseq 3:159 or |
| REMARK | 3 | | : resseq 164:209 or resseq 569:575) |
| REMARK | 3 | SELECTION | : chain G and (resseq 3:159 or |
| REMARK | 3 | | : resseq 164:209 or resseq 569:575) |
| REMARK | 3 | ATOM PAIRS NUMBER | : 1737 |
| REMARK | 3 | RMSD | : 0.057 |
| REMARK | 3 | NCS OPERATOR: 4 | |
| REMARK | 3 | REFERENCE SELECTION | : chain A and (resseq 3:159 or |
| REMARK | 3 | | : resseq 164:209 or resseq 569:575) |
| REMARK | 3 | SELECTION | : chain I and (resseq 3:159 or |
| REMARK | 3 | | : resseq 164:209 or resseq 569:575) |
| REMARK | 3 | ATOM PAIRS NUMBER | : 1737 |
| REMARK | 3 | RMSD | : 0.061 |
| REMARK | 3 | NCS OPERATOR: 5 | |
| REMARK | 3 | REFERENCE SELECTION | : chain A and (resseq 3:159 or |
| REMARK | 3 | | : resseq 164:209 or resseq 569:575) |
| REMARK | 3 | SELECTION | : chain K and (resseq 3:159 or |
| REMARK | 3 | | : resseq 164:209 or resseq 569:575) |
| REMARK | 3 | ATOM PAIRS NUMBER | : 1737 |
| REMARK | 3 | RMSD | : 0.068 |
| REMARK | 3 | NCS OPERATOR: 6 | |
| REMARK | 3 | REFERENCE SELECTION | : chain A and (resseq 3:159 or |
| REMARK | 3 | | : resseq 164:209 or resseq 569:575) |
| REMARK | 3 | SELECTION | : chain M and (resseq 3:159 or |
| REMARK | 3 | | : resseq 164:209 or resseq 569:575) |
| REMARK | 3 | ATOM PAIRS NUMBER | : 1737 |
| REMARK | 3 | RMSD | : 0.058 |
| REMARK | 3 | NCS OPERATOR: 7 | |
| REMARK | 3 | REFERENCE SELECTION | : chain A and (resseq 3:159 or |
| REMARK | 3 | | : resseq 164:209 or resseq 569:575) |
| REMARK | 3 | SELECTION | : chain O and (resseq 3:159 or |
| REMARK | 3 | | : resseq 164:209 or resseq 569:575) |
| REMARK | 3 | ATOM PAIRS NUMBER | : 1737 |
| REMARK | 3 | RMSD | : 0.058 |
| REMARK | 3 | NCS GROUP: 2 | |
| REMARK | 3 | NCS OPERATOR: 1 | |
| REMARK | 3 | REFERENCE SELECTION | : chain B and (resseq 5:81 or resseq |
| REMARK | 3 | | : 93:119 or resseq 600:602) |
| REMARK | 3 | SELECTION | : chain D and (resseq 5:81 or resseq |
| REMARK | 3 | | : 93:119 or resseq 600:602) |
| REMARK | 3 | ATOM PAIRS NUMBER | : 857 |
| REMARK | 3 | RMSD | : 0.068 |
| REMARK | 3 | NCS OPERATOR: 2 | |
| REMARK | 3 | REFERENCE SELECTION | : chain B and (resseq 5:81 or resseq |
| REMARK | 3 | | : 93:119 or resseq 600:602) |
| REMARK | 3 | SELECTION | : chain F and (resseq 5:81 or resseq |
| REMARK | 3 | | : 93:119 or resseq 600:602) |
| REMARK | 3 | ATOM PAIRS NUMBER | : 857 |
| REMARK | 3 | RMSD | : 0.063 |
| REMARK | 3 | NCS OPERATOR: 3 | |
| REMARK | 3 | REFERENCE SELECTION | : chain B and (resseq 5:81 or resseq |
| REMARK | 3 | | : 93:119 or resseq 600:602) |
| REMARK | 3 | SELECTION | : chain H and (resseq 5:81 or resseq |
| REMARK | 3 | | : 93:119 or resseq 600:602) |
| REMARK | 3 | ATOM PAIRS NUMBER | : 857 |
| REMARK | 3 | RMSD | : 0.069 |
| REMARK | 3 | NCS OPERATOR: 4 | |
| REMARK | 3 | REFERENCE SELECTION | : chain B and (resseq 5:81 or resseq |
| REMARK | 3 | | : 93:119 or resseq 600:602) |
| REMARK | 3 | SELECTION | : chain J and (resseq 5:81 or resseq |
| REMARK | 3 | | : 93:119 or resseq 600:602) |
| REMARK | 3 | ATOM PAIRS NUMBER | : 857 |
| REMARK | 3 | RMSD | : 0.072 |
| REMARK | 3 | NCS OPERATOR: 5 | |
| REMARK | 3 | REFERENCE SELECTION | : chain B and (resseq 5:81 or resseq |
| REMARK | 3 | | : 93:119 or resseq 600:602) |
| REMARK | 3 | SELECTION | : chain L and (resseq 5:81 or resseq |
| REMARK | 3 | | : 93:119 or resseq 600:602) |
| REMARK | 3 | ATOM PAIRS NUMBER | : 857 |
| REMARK | 3 | RMSD | : 0.070 |
| REMARK | 3 | NCS OPERATOR: 6 | |
| REMARK | 3 | REFERENCE SELECTION | : chain B and (resseq 5:81 or resseq |
| REMARK | 3 | | : 93:119 or resseq 600:602) |
| REMARK | 3 | SELECTION | : chain N and (resseq 5:81 or resseq |
| REMARK | 3 | | : 93:119 or resseq 600:602) |
| REMARK | 3 | ATOM PAIRS NUMBER | : 857 |
| REMARK | 3 | RMSD | : 0.068 |
| REMARK | 3 | NCS OPERATOR: 7 | |
| REMARK | 3 | REFERENCE SELECTION | : chain B and (resseq 5:81 or resseq |
| REMARK | 3 | | : 93:119 or resseq 600:602) |
| REMARK | 3 | SELECTION | : chain P and (resseq 5:81 or resseq |
| REMARK | 3 | | : 93:119 or resseq 600:602) |
| REMARK | 3 | ATOM PAIRS NUMBER | : 857 |
| REMARK | 3 | RMSD | : 0.065 |
| REMARK | 3 | | |

TABLE 4
hIL-21, chain I, (SEQ ID NO: 1) interactions with the chain
(chain R) of hIL-21Rα (SEQ ID NO: 14). A distance cut-off
of 4.0 Å was used. The contacts were identified by the
CONTACT computer software program of the CCP4 suite [7].
In the last column “***” indicates a strong possibility
for a hydrogen bond at this contact (distance <3.3 Å)
as calculated by CONTACT, “*” indicates a weak possibility
(distance >3.3 Å). Blank indicates that the program considered
there to be no possibility of a hydrogen bond. Hydrogen-bonds
are specific between a donor and an acceptor, are typically
strong, and are easily identifiable.

| hIL-21 | | | hIL-21Rα | | | | |
|---|---|---|---|---|---|---|---|
| Res. | Res. # | Atom | Res. | Res. # | Atom | Distance | Possibly |
| Type | and Chain | name | Type | and Chain | name | [Å] | H-bond |
| Arg | 34 I | O | Asp | 74 R | CB | 3.67 | |
| Arg | 34 I | CD | Asp | 74 R | O | 3.75 | |
| Arg | 34 I | NE | Asp | 74 R | O | 3.39 | * |
| Arg | 34 I | CZ | Asp | 74 R | O | 3.61 | |
| Arg | 34 I | NH1 | Gln | 35 R | OE1 | 3.79 | * |
| | | | Ile | 76 R | CG1 | 3.30 | |
| Arg | 34 I | NH2 | Gln | 37 R | OE1 | 3.31 | * |
| Ile | 37 I | CG2 | Ala | 73 R | O | 3.98 | |
| | | | Ala | 73 R | C | 3.77 | |
| | | | Asp | 74 R | N | 3.65 | |
| | | | Asp | 74 R | CA | 3.84 | |
| | | | Asp | 74 R | CB | 3.78 | |
| | | | Ala | 73 R | CB | 3.82 | |
| Ile | 37 I | CD1 | Leu | 96 R | CD1 | 3.99 | |
| | | | Ala | 98 R | CB | 3.84 | |
| Arg | 38 I | CA | Asp | 74 R | OD1 | 3.69 | |
| Arg | 38 I | CB | Asp | 74 R | OD1 | 3.49 | |
| Arg | 38 I | CD | Asp | 74 R | OD1 | 3.43 | |
| Arg | 38 I | NE | Asp | 74 R | OD2 | 3.62 | * |
| | | | Asp | 74 R | CG | 3.35 | |
| | | | Asp | 74 R | OD1 | 2.49 | *** |
| Arg | 38 I | CZ | Asp | 74 R | OD2 | 3.73 | |
| | | | Asp | 74 R | CG | 3.90 | |
| | | | Asp | 74 R | OD1 | 3.33 | |
| Arg | 38 I | NH2 | Asp | 74 R | OD2 | 2.99 | *** |
| | | | Asp | 74 R | CG | 3.54 | |
| | | | Asp | 74 R | OD1 | 3.33 | * |
| Arg | 40 I | NE | Ser | 192 R | OG | 3.79 | * |
| Arg | 40 I | CZ | Ser | 192 R | CB | 3.72 | |
| | | | Ser | 192 R | OG | 3.62 | |
| Arg | 40 I | NH1 | Ser | 192 R | CA | 3.20 | |
| | | | Ser | 192 R | CB | 2.68 | |
| | | | Ser | 192 R | OG | 2.72 | *** |
| | | | Ser | 192 R | N | 3.29 | *** |
| Gln | 41 I | CD | Met | 72 R | CE | 3.89 | |
| | | | Ala | 73 R | N | 3.64 | |
| | | | Ala | 73 R | CB | 3.93 | |
| Gln | 41 I | OE1 | Met | 72 R | CA | 3.30 | |
| | | | Met | 72 R | C | 3.57 | |
| | | | Met | 72 R | CB | 3.29 | |
| | | | Met | 72 R | CE | 3.04 | |
| | | | Ala | 73 R | N | 2.88 | *** |
| | | | Ala | 73 R | CA | 3.96 | |
| | | | Ala | 73 R | CB | 3.87 | |
| Gln | 41 I | NE2 | Ala | 73 R | N | 3.74 | * |
| | | | Tyr | 12 R | OH | 3.39 | * |
| | | | Ala | 73 R | CB | 3.62 | |
| | | | Tyr | 193 R | OH | 3.02 | *** |
| Asp | 44 I | OD2 | Lys | 136 R | CE | 3.90 | |
| | | | Lys | 136 R | NZ | 3.04 | *** |
| Ile | 45 I | CG1 | Tyr | 131 R | OH | 3.72 | |
| Ile | 45 I | CD1 | Met | 72 R | CE | 3.49 | |
| Gln | 48 I | CG | Tyr | 131 R | OH | 3.93 | |
| | | | Tyr | 131 R | CZ | 3.63 | |
| | | | Tyr | 131 R | CE1 | 3.61 | |
| Gln | 48 I | CD | Tyr | 131 R | OH | 3.84 | |
| | | | Tyr | 131 R | CZ | 3.41 | |
| | | | Tyr | 131 R | CD2 | 3.72 | |
| | | | Tyr | 131 R | CE2 | 3.35 | |
| | | | Tyr | 131 R | CE1 | 3.81 | |
| Gln | 48 I | OE1 | Tyr | 131 R | OH | 3.45 | * |
| | | | Tyr | 131 R | CZ | 3.36 | |
| | | | Tyr | 131 R | CD2 | 3.93 | |
| | | | Tyr | 131 R | CE2 | 3.21 | |
| Gln | 48 I | NE2 | Tyr | 131 R | CG | 3.76 | |
| | | | Tyr | 131 R | CD2 | 3.48 | |
| | | | Tyr | 131 R | CE2 | 3.61 | |
| Tyr | 52 I | OH | Pro | 128 R | CB | 3.59 | |
| Arg | 94 I | NH2 | His | 70 R | CE1 | 3.84 | |
| | | | His | 70 R | NE2 | 3.37 | * |
| | | | Phe | 69 R | CZ | 3.73 | |
| Ile | 95 I | CG1 | Ala | 129 R | CB | 3.93 | |
| Ile | 95 I | CD1 | Ala | 129 R | CA | 3.60 | |
| | | | Ala | 129 R | CB | 3.77 | |
| | | | Pro | 128 R | O | 3.82 | |
| | | | Pro | 128 R | C | 3.77 | |
| | | | Ala | 129 R | N | 3.62 | |
| | | | Pro | 128 R | CB | 3.97 | |
| Val | 98 I | CG2 | His | 70 R | CD2 | 3.80 | |
| | | | His | 70 R | NE2 | 3.74 | |
| Ser | 99 I | OG | Tyr | 131 R | OH | 3.21 | *** |
| Lys | 102 I | CA | Met | 72 R | SD | 3.91 | |
| Lys | 102 I | O | Met | 72 R | SD | 3.70 | |
| Lys | 102 I | CB | Met | 72 R | SD | 3.49 | |
| | | | Met | 72 R | CE | 3.97 | |
| Lys | 102 I | CG | Met | 72 R | SD | 3.84 | |
| Lys | 102 I | CD | Met | 72 R | SD | 4.00 | |
| | | | Met | 72 R | CE | 3.98 | |
| Lys | 102 I | CE | His | 70 R | O | 3.33 | |
| | | | His | 70 R | C | 3.71 | |
| | | | Phe | 69 R | O | 3.62 | |
| | | | Phe | 71 R | O | 3.82 | |
| Lys | 102 I | NZ | Phe | 69 R | O | 3.09 | *** |
| | | | Phe | 71 R | O | 3.51 | * |
| | | | Met | 132 R | CE | 3.59 | |
| Arg | 105 I | CA | Glu | 40 R | OE1 | 3.56 | |
| | | | Glu | 40 R | CD | 3.84 | |
| | | | Glu | 40 R | OE2 | 3.77 | |
| Arg | 105 I | C | Glu | 40 R | OE1 | 3.85 | |
| | | | Glu | 40 R | OE2 | 3.89 | |
| Arg | 105 I | CB | Glu | 40 R | CD | 3.82 | |
| | | | Glu | 40 R | OE2 | 3.47 | |
| Arg | 105 I | CG | Glu | 40 R | CB | 3.87 | |
| | | | Glu | 40 R | OE1 | 3.77 | |
| | | | Glu | 40 R | CG | 3.70 | |
| | | | Glu | 40 R | CD | 3.28 | |
| | | | Glu | 40 R | OE2 | 3.17 | |
| Arg | 105 I | NE | Leu | 41 R | CD2 | 3.90 | |
| | | | Met | 72 R | CG | 3.61 | |
| | | | Met | 72 R | SD | 4.00 | |
| Arg | 105 I | CZ | Glu | 40 R | OE2 | 3.63 | |
| | | | Asp | 75 R | OD1 | 3.95 | |
| | | | Asp | 75 R | OD2 | 3.96 | |
| Arg | 105 I | NH1 | Glu | 40 R | CG | 3.05 | |
| | | | Glu | 40 R | CD | 3.03 | |
| | | | Glu | 40 R | OE2 | 2.41 | *** |
| Arg | 105 I | NH2 | Leu | 41 R | CD1 | 3.83 | |
| | | | Asp | 75 R | CG | 3.11 | |
| | | | Asp | 75 R | OD1 | 2.64 | *** |
| | | | Asp | 75 R | OD2 | 2.87 | *** |
| Lys | 106 I | N | Glu | 40 R | OE1 | 3.15 | *** |
| | | | Glu | 40 R | CD | 3.66 | |
| | | | Glu | 40 R | OE2 | 3.36 | * |
| Lys | 106 I | O | Tyr | 38 R | CD2 | 3.71 | |
| | | | Tyr | 38 R | CE2 | 3.59 | |
| | | | Glu | 40 R | OE2 | 3.62 | * |
| Lys | 106 I | CB | Tyr | 38 R | CD2 | 3.93 | |
| Lys | 106 I | CG | Tyr | 38 R | CE2 | 3.97 | |
| Pro | 107 I | O | Tyr | 38 R | CE2 | 3.94 | |
| | | | Tyr | 38 R | OH | 3.91 | * |
| Pro | 108 I | CG | Asp | 74 R | OD2 | 3.43 | |
| | | | Asp | 74 R | CG | 3.93 | |
| Ser | 109 I | N | Tyr | 38 R | OH | 3.54 | * |

### Example 3

In order to design mutants of NNC 0114-0005 which bind to the epitope described herein, the Kabat defined CDR-loops for anti-IL21 Fab NNC 0114-0009 are analysed. Fab NNC 0114-0009 designates the Fab fragments of NNC 0114-0005.

CDR-regions comprise the following residues (CDR-residues):
CDR_L1: R24 A25 S26 Q27 S28 V29 S30 S31 S32 Y33 L34 A35
CDR_L2: G51 A52 S53 S54 R55 A56 T57
CDR_L3: Q90 Q91 Y92 G93 S94 W95 T96
CDR_H1: S31 Y32 G33 M34 H35
CDR_H2: F50 I51 W52 Y53 D54 G55 S56 D57 K58 Y59 Y60 A61 D62 S63 V64 K65 G66
CDR_H3: D99 G100 D101 S102 S103 D104 W105 Y106 G107 D108 Y109 Y110 F111 G112 M113 D114 V115

The paratope defined using a 4 Å distance cut-off is determined from the crystal structure of the Fab NNC 0114-0009:hIL-21 complex. The paratope is determined to comprise the following residues:

In CDR_L1: Q27 S28 V29 S30 S32 Y33

In CDR_L2: None

In CDR_L3: Q91 Y92 G93 S94 W95

In CDR_H1: None

In CDR_H2: W52 S56 D57 Y59 Y60

In CDR_H3: D99 D101 S102 S103 D104 W105 Y106 G107 D108 Y109 F111

Thus, CDR-residues not included in the paratope are the following (in total 38):

In CDR_L1: R24 A25 S26 S31 L34 A35

In CDR_L2: G51 A52 S53 S54 R55 A56 T57

In CDR_L3: Q90 T96

In CDR_H1: S31 Y32 G33 M34 H35

In CDR_H2: F50 I51 Y53 D54 G55 K58 A61 D62 S63 V64 K65 G66

In CDR_H3: G100 Y110 G112 M113 D114 V115

Among the 38 non-paratope CDR-residues 13 were selected as potential mutation sites. The selection was based on inspection of the crystal structure. Extensively buried residues and residues for which the side chains appeared to be involved in several important interactions were deselected. The identified potential mutation sites are listed in Table 5. Specific mutations (Table 5) at these sites were chosen such that no or minimal effect on the protein structure would result.

TABLE 5
Selected mutation sites and suggested mutations. Each of the individual
mutations shown in this table represent different embodiments
of the present invention, i.e. monoclonal antibodies having the
ability to bind to essentially the same epitope as the hIL-21Rα
receptor. Antibodies according to the invention may also comprise
two or more of the mutations shown in this table. It follows that
variant antibodies according to the invention can only comprise
one mutation in a specific position.

| | | | | Muta- | NNCD | NNCD |
|---|---|---|---|---|---|---|
| CDR | # | Chain | Name | tions | no. mAb | no. Fab |
| H1 | 31 | H | SER | S31A | 0114-0000-0038 | 0114-0000-0051 |
| H2 | 53 | H | TYR | Y53F | | |
| H2 | 61 | H | ALA | A61S | | |
| H2 | 63 | H | SER | S63T | | |
| H2 | 63 | H | SER | S63A | | |
| H2 | 65 | H | LYS | K65R | | |
| L1 | 24 | L | ARG | R24K | | |
| L1 | 26 | L | SER | S26T | | |
| L1 | 31 | L | SER | S31T | | |
| L1 | 31 | L | SER | S31A | | |
| L2 | 53 | L | SER | S53A | 0114-0000-0039 | 0114-0000-0045 |
| L2 | 53 | L | SER | S53T | 0114-0000-0040 | 0114-0000-0046 |
| L2 | 54 | L | SER | S54T | 0114-0000-0042 | 0114-0000-0048 |
| L2 | 54 | L | SER | S54A | 0114-0000-0041 | 0114-0000-0047 |
| L2 | 55 | L | ARG | R55K | 0114-0000-0043 | 0114-0000-0049 |
| L2 | 57 | L | THR | T57S | 0114-0000-0044 | 0114-0000-0050 |
| L3 | 96 | L | THR | T96S | | |

This example describes one method applicable for designing antibodies according to the invention based on the information contained in the crystal structure of Fab NNC 0114-0009:IL-21. It follows that several other approaches can be taken in designing ligands according to the invention.

One approach could be e.g. to design a ligand essentially comprising the paratope of Fab0009 except that one or more conservative substitutions can be made.

Another approach could be to design an IL-21 ligand based on the structure of the binding interface between IL-21 and IL-21Rα. This ligand could be in the form of e.g. an antibody or an IL-21Rα variant/mimic that essentially retains the structure of said IL-21Rα binding interface.

It follows that one or more of such approaches can be combined.

Autoimmune disorders and other immune related disorders can be treated with e.g. therapeutic human monoclonal antibodies. However, said monoclonal antibodies may be immunogenic and give rise to the formation of anti-antibodies. It is conceivable that such anti-antibodies bind to areas of the therapeutic antibodies directly involved in binding to the target molecule. If such immunogenic sites, leading to development of anti-antibodies against NNC 0114-0005, are recognized and characterized, the detailed description of the paratope for the antibody NNC 0114-0005 derived from the 3-dimensional structure of the Fab NNC 0114-0009:IL-21 complex provides a possibility for rationally designing variants of NNC 0114-0005 that will retain high-affinity binding to hIL-21, but potentially are less immunogenic. Alternatively, variants of NNC 0114-0005 may be designed in such a way that unwanted binding to specific anti-antibodies is reduced or prevented. It is thus possible to use the crystal structure information to provide improved versions of NNC 0114-0005.

The provision of the crystal structure of this Fab fragment as well as its paratope also provides the possibility of e.g. replacing residues therein that could potentially result in improvement with respect to stability, solubility or other chemical or physical properties of a molecule comprising this paratope while maintaining its biological functionality including high-affinity binding to IL21. Stability can e.g. be improved by reducing aggregation, self association, fragmentation, and disulfide formation/exchange. Improved solubility might include properties leading to improved viscosity.

The provision of this crystal structure furthermore provides a possibility of providing a modified molecule having reduced tendency to undergo e.g. deamidation, isomerization and/or oxidation and thereby improving the physical/chemical stability of a molecule comprising this paratope while maintaining its biological functionality including high-affinity to IL-21.

One example of potential stability improving mutations in the antibody NNC 0114-0005 is the elimination of potential oxidation sites by mutation of Methionine residues. One specific example of such a mutation is the change of the Methionine in position 34 in the heavy chain (SEQ ID No 10) to an amino acid with similar properties, e.g. Isoleucine.

One example of potential stability improving mutations in the antibody NNC 0114-0005 is elimination of potential hot-spots (DX-motifs, e.g. DG- and DS-motifs) for isomerisation of Aspartate residues. Such potentially labile DX-motifs can be eliminated by appropriate mutation of one or both of the constituent D or X residues. One specific example af such a mutation is the change of the Aspartate (present in a DG motif) in position 54 in the heavy chain (SEQ ID No 10) to an amino acid with similar properties, e.g. Glutamate. A second specific example af such a mutation is the change of the Aspartate (present in a DG motif) in position 99 in the heavy chain (SEQ ID No 10) to an amino acid with similar properties, e.g. Glutamate. An third specific example af such a mutation is the change of the Aspartate (present in a DS motif) in position 101 in the heavy chain (SEQ ID No 10) to an amino acid with similar properties, e.g. Glutamate.

One example of potential stability improving mutations in the antibody NNC 0114-0005 is elimination of potential hot-spots (NX-motifs, e.g. NG- or NS-motifs) for deamidation of Asparagine residues. Such potentially labile NX-motifs can be eliminated by appropriate mutation of one or both of the constituent N or X residues. One specific example af such a mutation is the change of the Asparagine (present in a NS motif) in position 74 in the heavy chain (SEQ ID No 10) to an amino acid with similar properties, e.g. Glutamine. N74

### Example 4

### Studies of the Interaction Between Anti hIL-21 Antibodies and hIL-21 by Surface Plasmon Resonance (SPR)

All binding studies were performed on a Biacore T100 instrument that measures molecular interactions in real time through surface plasmon resonance. Experiments were run at 25° C. and the samples were stored at 15° C. in the sample compartment. The signal (RU, response units) reported by the Biacore is directly correlated to the mass on the individual sensor chip surfaces in four serial flow cells.

Anti-hFc monoclonal or anti-mFc polyclonal antibody from Biacore human or mouse Fc capture kits were immobilized onto flow cells of a CM4 sensor chip according to the manufacturer's instructions. The final immobilization level of capture antibody was approximately 2,000 RU in one experiment. Capture of the human anti-IL-21 antibodies NNC 0114-0005, NNC 0114-0015 and rat anti-IL-21 antibody NNC 0114-0019 was conducted by diluting each antibody to 0.25-1 μg/ml into running buffer (10 mM Hepes 0.3 M NaCl, 5 mM CaCl2, 0.05% surfactant P20, pH 8.0 containing 1 mg/ml BSA) and injected at 10 μl/min for 180s in one of flow cells 2-4, creating a reference surface in flow cell 1 with only anti-Fc antibody immobilized. The final capture level of test antibodies typically ranged from approximately 20 to 160 RU in one experiment. Binding of hIL-21 protein was conducted by injecting analyte over all flow cells to allow for comparative analyses of binding to different captured anti-IL-21 antibodies relative to binding to the reference flow cell. hIL-21 protein was diluted serially 1:2 to 0.008-2 nM into running buffer, injected at 50 μl/min for 600s and allowed to dissociate for 600 or 3600 s. The CM4 surface was regenerated after each injection cycle of analyte via two injections of 20 mM HCl at 50 μl/min. This regeneration step removed the anti-IL-21 antibody and any bound IL-21 from the immobilized capture antibody surface, and allowed for the subsequent binding of the next interaction sample pair. The regeneration procedure did not remove the directly immobilized anti-human-Fc capture antibody from the chip surface.

In order to obtain kinetic data, such as ka (association rate), kd (dissociation rate) and KD (binding affinity), data analysis was performed using the Biacore T100 evaluation software 2.0.3. No significant non-specific binding to the reference control surface was observed. Binding curves were processed by double referencing (subtraction of reference surface signals as well as blank buffer injections over captured anti-IL-21 antibodies). This allowed correction for instrument noise, bulk shift and drift during sample injections.

NNC 0114-0005 and 0114-0015 interact with hIL-21 at very high association rates resulting in mass transport limiting conditions and exact kinetic values could initially not be obtained. After assay modifications in flow rate, dissociation times, surface density and new hardware with better sensitivity as described in Example 11, new experiments where performed. Results then showed that human IL-21 binds to NNC 0114-0005 and 0114-0015 with an average KD of ≦1 pM, dissociation rates of 1 E-5 s⁻¹ and association rates of 3-4 E+7 (Ms)⁻¹. These two antibodies thus share similar binding properties to human IL-21.

Human IL-21 binds to NNC 0114-0019 with average >10-fold lower affinity due to a slower association rate, 5-6 E+7 (Ms)⁻¹ compared to that of 0114-0005 and 0114-0015. Results are based on 1-3 different experiments for each antibody. Relative standard errors of parameters ka and kd were 0.2-1.5% in the individual experiments.

TABLE 6
Results from individual experiments of binding constants ka
(association rate), kd (dissociation rate) and KD (equilibrium
dissociation constant) for the interaction of human IL-21
to monoclonal antibodies 0114-0005, 0015 and 0019.

| | Experi- | ka | kd | KD | RSE | RSE |
|---|---|---|---|---|---|---|
| Ligand | ment no | (1/Ms) | (1/s) | (M) | ka (%) | kd (%) |
| 0005 | 1 | 1.6E+07 | 9.5E−06 | 5.8E−13 | 0.4 | 0.3 |
| 0005 | 1 | 1.5E+07 | 1.1E−05 | 7.2E−13 | 0.4 | 0.3 |
| 0005 | 2 | 6.6E+07 | 1.1E−05 | 1.7E−13 | 1.5 | 0.4 |
| 0019 | 3 | 2.0E+06 | 1.3E−05 | 6.3E−12 | 0.2 | 0.3 |
| 0019 | 4 | 1.7E+06 | 1.0E−05 | 6.1E−12 | 0.4 | 0.3 |
| 0019 | 5 | 1.9E+06 | 1.0E−05 | 5.2E−12 | 0.4 | 0.3 |
| 0015 | 6 | 5.5E+07 | 6.8E−06 | 1.2E−13 | 0.4 | 0.5 |
| 0015 | 6 | 4.9E+07 | 8.9E−06 | 1.8E−13 | 0.8 | 0.9 |
| 0015 | 6 | 4.3E+07 | 1.2E−05 | 2.8E−13 | 1.1 | 0.7 |

### Example 5

### Bioactivity in NK-92 Assay

A bioassay was developed with the NK-92 cell line that expresses the IL-21Rα endogenously and is dependent on IL-2 or IL-21 for growth. This assay can be used for in vitro determination of the potency of anti-IL-21 antibodies. The NK-92 cell line is a human suspension lymphoblast derived from peripheral blood mononuclear cells, and it can be purchased from ATCC/LGC Promochem. The neutralization of IL-21 by anti-IL-21 is measured by growth inhibition via addition of alamarBlue® (a cell viability indicator).

During normal maintenance culture the NK-92 cells are kept viable by IL-2. For the assay NK-92 cells are washed and plated out in 96 well plates (Matrix Technology) at a density of 1.6×10⁵ cells/ml (equal to 12.800 cells per well), the cells are seed into the plates. The cells are stimulated with recombinant human IL-21 at a fixed concentration of 5431 pg/ml. Serial dilutions of Anti-IL-21 antibodies prepared in assay media ranging from 0-12,800 pg/ml are added in triplicates in three different positions on three individual plates. The cells are incubated for 3 days with CO₂ in an incubator from Heto Holten. On day three 10 μl alamarBlue® (Biosource) is added and fluorescence is measured after 5 hours on a Synergy instrument (Bio Tek).

Data are analyzed in BioCalc (MicroLex) as combined potency (U/ml) (geometric mean of the three independent positions) correlated to the protein concentration (mg/ml) and reported as specific activity (U/mg). The specific activity for NNC114-0005 was determined to be 1.0 U/mg.

### Example 6

### B Cell Proliferation and Maturation Assays

To test the effect of the anti-IL-21 antibody in a biologically relevant setting three functional assays were established where relevant IL-21 biology was studied in primary human cells.

Stimulation with a combination of Anti-CD40 antibody and recombinant IL-21 induces proliferation of primary B cells and B cells maturation as measured by the frequency of plasma blasts with a CD19+CD27highCD38high phenotype. The Anti-IL-21 antibody(ies) were able to prevent both proliferation and maturation.

The relevance of B cells to chronic inflammatory disease has been described in the literature as well as by the clinical effect of B-cell depletion with Rituximab in e.g. rheumatoid arthritis. In the literature B cells were shown to play an important role in driving chronic inflammation (Dorner T et al (2009) Arthritis Res. Therapy), both as antigen presenting cells as well as producers of (auto)antibodies. IL-21 induces B cell proliferation (when combined with CD40 co-stimulation), immunoglobulin (Ig) class switching to particular IgG1 and IgG3, and differentiation of activated B cells to Ig-producing plasma cells (Ozaki, K. et al., Science, 2002; Ettinger R. J. et al., J Immunol, 2005; Kuchen, S., et al., J Immunol, 2007; Ettinger, R. et al., Immunol Rev, 2008; Leonard, W. J. et al. Nat. Rev. Immunol. 2005). Neutralization of IL-21 activity is therefore expected to reduce B cell differentiation and thus potentially decrease B cell immune-stimulating properties and autoantibody production in autoimmune patients.

Blood bags were obtained from healthy human volunteers and PBMCs were isolated from 50 ml of heparinised peripheral blood by Ficoll-Paque™ Plus (GE Healthcare) gradient centrifugation. Blood was diluted to 100 ml in phosphate-buffered saline (PBS) at room temperature and 35 ml aliquots were distributed into 50 ml conical tubes carefully overlaying 14 ml of Ficoll-Paque™ Plus (Ge Healthcare) at room temperature. The tubes were spun for 25 minutes at 1680 rpm (600×g) at room temperature without brake. The PBMC interface layer was removed carefully and washed twice with PBS containing 2% FCS. B cells were isolated by negative selection using EasySep human B Cell enrichment Kit (StemCell Technologies SERL, Grenoble, France). A small sample of the purified B cells was tested for purity by FACS analysis and found to be >95-97% pure in all experiments.

B cells were cultured in RPMI-1640 media (InVitrogen) supplemented with heat inactivated foetal calf serum (FCS) (Gibco) or Healthy human serum (HS) (Sigma), and Penicillin/Streptomycin (Gibco). Purified human B cells were plated at 50,000 cells/well in a 96-well U-bottom tissue culture plate (BD Biosciences). The cells were treated with or without 0.1 μg/ml anti-CD40 (goat anti-human CD40 polyclonal; R&D Systems), plus a titration of recombinant human IL-21 (Novo Nordisk NS) prepared as a 1:3 serial dilution. The plate of cells was then incubated for 3 days at 37° C. and 5% CO₂ in a humidified incubator. After three days, the cells were pulsed with 1 μCi/well of [3H]-Thymidine (Perkin Elmer Life Sciences). After 16 hours, the cells were harvested onto UniFilter-96 GF/C filter plates (Packard, Perkin Elmer) and the amount of [3H]-Thymidine incorporation was quantitated using a TopCount NXT (Perkin Elmer Life Sciences). The effective concentration of IL-21 required for induction of 50% and 90% maximum proliferation (EC50 and EC90, respectively) were calculated using the GraphPad Prism v5.0 software (GraphPad Inc) and the sigmoidal dose-response (variable slope) equation.

To determine the neutralising potential of the anti-IL-21 mAb, B cells were plated at 50.000 cells per well in a 96-well U-bottom tissue culture plate. Cells were treated with 0.1 μg/ml anti-CD40 (R&D Systems), 50 ng/ml (3.21 nM) recombinant human IL-21 and a titration of anti IL-21 mAb. The cells were incubated for 3 days at 37° C. and 5% CO2 in a humidified incubator. After three days, the cells were pulsed with 1 μCi/well of [3H]-Thymidine (Perkin Elmer Life Sciences) for the last 20 hours. The cells were harvested onto UniFilter-96 GF/C filter plates (Packard Instruments, Perkin Elmer) and the amount of [3H]-thymidine incorporation was quantified using a TopCount NXT (Perkin Elmer). The inhibitive concentration of anti IL-21 mAb required for reducing proliferation by 50% (IC50) was calculated using the GraphPad Prism v5.0 software (GraphPad Inc.) and the sigmoidal dose-response (variable slope, 4-parameters) equation.

The IC50 for NNC114-0005 was shown to be in the low nanomolar range in these experiments.

IC50

| mAb | IC50 (nM) a) +/− SD |
|---|---|
| NNC114-0005 | 1.229 +/− 0.247 |

a) IC50 based on 4 individual donors

The inhibitory effect of anti-IL-21 antibodies on proliferation was further measured by flow cytometry using carboxyfluorescein succinimidyl ester (CFSE). Purified B cells were stained with 0.2 μM CFSE and plated at 50.000 cells per well in a 96-well U-bottom tissue culture plate. Cells were stimulated with 0.1 μg/ml anti-CD40 (R&D Systems), 50 ng/ml (3.21 nM) recombinant human IL-21 and a 10-fold titration of NNC114-0005 and NNC114-0015. Start concentration of NNC114-0005 and NNC114-0015 was 50 and 500 ng/ml respectively. The cells were incubated for 6 days at 37° C. and 5% CO2 in a humidified incubator. Proliferation was measured by gating on live B cells after staining with LIVE/DEAD Fixable Dead cell stain and for CD19 surface expression.

### Proliferation

| mAb | No mAb | 500 ng/ml | 50 ng/ml | 5 ng/ml | 0.5 ng/ml |
|---|---|---|---|---|---|
| NNC114- | 84% | — | 27% | 79% | 83% |
| 0005 | | | | | |
| NNC114- | 84% | 27% | 75% | 82% | — |
| 0015 | | | | | |

To further substantiate the important role of NNC114-0005 on B cells the effect on B cell maturation was measured by flow cytometry. Purified B cells were plated at 50.000 cells per well in a 96-well U-bottom tissue culture plate. Cells were stimulated with 0.1 μg/ml anti-CD40 (R&D Systems), 50 ng/ml (3.21 nM) recombinant human IL-21 and a 3-fold titration of NNC114-0005 and NNC114-0015. Start concentration of NNC114-0005 and NNC114-0015 was 1 and 10 ng/ml respectively. The cells were incubated for 6 days at 37° C. and 5% CO2 in a humidified incubator. Live B cells were gated after staining with LIVE/DEAD Fixable Dead cell stain and CD19 surface expression. The IL-21/anti-CD40 induced B cell maturation into plasma blasts and plasma cells was followed by staining the B cells for expression of CD27, CD38 and CD138. The inhibitory effect of NNC114-0005 and -0015 on plasma blast and plasma cell maturation was identified by gating on the CD19+CD27highCD38high and CD19+CD27highCD138+phenotype respectively.

Percentage of CD19+CD27highCD38high plasmablasts

| | No | 1 | 3 | 10 | 30 | 100 | 300 | 1000 |
|---|---|---|---|---|---|---|---|---|
| mAb | mAb | ng/ml | ng/ml | ng/ml | ng/ml | ng/ml | ng/ml | ng/ml |
| NNC114- | 9.4 | 9.1 | 10.7 | 8.2 | 0 | 0 | ND | ND |
| 0005 | | | | | | | | |
| NNC114- | 9.3 | ND | ND | 9.3 | 12.6 | 6.6 | 0 | 0 |
| 0015 | | | | | | | | |
| hIgG1 | 9.0 | ND | ND | 9.6 | 11.2 | 10.4 | 10.1 | 9.0 |

ND: not done

Percentage of CD138+ plasma cells (CD19+CD27highCD138+)

| | No | 1 | 3 | 10 | 30 | 100 | 300 | 1000 |
|---|---|---|---|---|---|---|---|---|
| mAb | mAb | ng/ml | ng/ml | ng/ml | ng/ml | ng/ml | ng/ml | ng/ml |
| NNC114- | 4.3 | 4.0 | 5.0 | 4.1 | 0 | 0 | ND | ND |
| 0005 | | | | | | | | |
| NNC114- | 3.3 | ND | ND | 5.0 | 4.7 | 4.1 | 0 | 0 |
| 0015 | | | | | | | | |
| hIgG1 | 4.3 | ND | ND | 3.7 | 6.0 | 4.6 | 4.0 | 4.0 |

ND: not done

### Example 7

### Epitope Mapping by HX-MS of mAbs NNC 0114-0005 and NNC 0114-0019 on hIL-21

### Introduction to HX-MS

The HX-MS technology exploits that hydrogen exchange (HX) of a protein can readily be followed by mass spectrometry (MS). By replacing the aqueous solvent containing hydrogen with aqueous solvent containing deuterium, incorporation of a deuterium atom at a given site in a protein will give rise to an increase in mass of 1 Da. This mass increase can be monitored as a function of time by mass spectrometry in quenched samples of the exchange reaction. The deuterium labelling information can be sub-localized to regions in the protein by pepsin digestion under quench conditions and following the mass increase of the resulting peptides.

One use of HX-MS is to probe for sites involved in molecular interactions by identifying regions of reduced hydrogen exchange upon protein-protein complex formation. Usually, binding interfaces will be revealed by marked reductions in hydrogen exchange due to steric exclusion of solvent. Protein-protein complex formation may be detected by HX-MS simply by measuring the total amount of deuterium incorporated in either protein members in the presence and absence of the respective binding partner as a function of time. The HX-MS technique uses the native components, ie protein and antibody or Fab fragment, and is performed in solution. Thus HX-MS provides the possibility for mimicking the in vivo conditions (for a recent review on the HX-MS technology, see Wales and Engen, Mass Spectrom. Rev. 25, 158 (2006)).

### Materials

Protein Batches Used were:

hIL-21: human recombinant IL-21, corresponding to residues 30-162 and with an N-terminal Methionine as residue 29.

mAb: NNC 0114-0005

mAb: NNC 0114-0019, clone number: 272.21.1.3.4.2 from WO2007/111714 (ATCC accession no. PTA-7142)

All proteins were buffer exchanged into PBS pH 7.4 before experiments.

### Methods: HX-MS Experiments

### Instrumentation and Data Recording

The HX experiments were automated by a Leap robot (H/D-x PAL; Leap Technologies Inc.) operated by the LeapShell software (Leap Technologies Inc.), which performed initiation of the deuterium exchange reaction, reaction time control, quench reaction, injection onto the UPLC system and digestion time control. The Leap robot was equipped with two temperature controlled stacks maintained at 20° C. for buffer storage and HX reactions and maintained at 2° C. for storage of protein and quench solution, respectively. The Leap robot furthermore contained a cooled Trio VS unit (Leap Technologies Inc.) holding the pre- and analytical columns, and the LC tubing and switching valves at 1° C. The switching valves of the Trio VS unit have been upgraded from HPLC to Microbore UHPLC switch valves (Cheminert, VICI AG). For the inline pepsin digestion, 100 μL quenched sample containing 200 pmol hIL-21 was loaded and passed over a Poroszyme® Immobilized Pepsin Cartridge (2.1×30 mm (Applied Biosystems)) placed at 20° C. using a isocratic flow rate of 200 μL/min (0.1% formic acid:CH₃CN 95:5). The resulting peptides were trapped and desalted on a VanGuard pre-column BEH C18 1.7 μm (2.1×5 mm (Waters Inc.)). Subsequently, the valves were switched to place the pre-column inline with the analytical column, UPLC-BEH C18 1.7 μm (2.1×100 mm (Waters Inc.)), and the peptides separated using a 9 min gradient of 15-35% B delivered at 200 μl/min from an AQUITY UPLC system (Waters Inc.). The mobile phases consisted of A: 0.1% formic acid and B: 0.1% formic acid in CH₃CN. The ESI MS data, and the separate data dependent MS/MS acquisitions (CID) and elevated energy (MS^{E}) experiments were acquired in positive ion mode using a Q-TOF Premier MS (Waters Inc.). Leucine-enkephalin was used as the lock mass ([M+H]⁺ ion at m/z 556.2771) and data was collected in continuum mode (For further description of the set-up, see Andersen and Faber, Int. J. Mass Spec., 302, 139-148 (2011))).

### Data Analysis

Peptic peptides were identified in separate experiments using standard CID MS/MS or MS^{E} methods (Waters Inc.). MS^{E} data were processed using BiopharmaLynx 1.2 (version 017). CID data-dependent MS/MS acquisition was analyzed using the MassLynx software and in-house MASCOT database.

HX-MS raw data files were subjected to continuous lock mass-correction. Data analysis, i.e., centroid determination of deuterated peptides and plotting of in-exchange curves, was performed using prototype custom software (HDX browser, Waters Inc.) and HX-Express ((Version Beta); Weis et al., J. Am. Soc. Mass Spectrom. 17, 1700 (2006)). All data were also visually evaluated to ensure only resolved peptide isotopic envelopes were subjected to analysis.

### Epitope Mapping Experiment

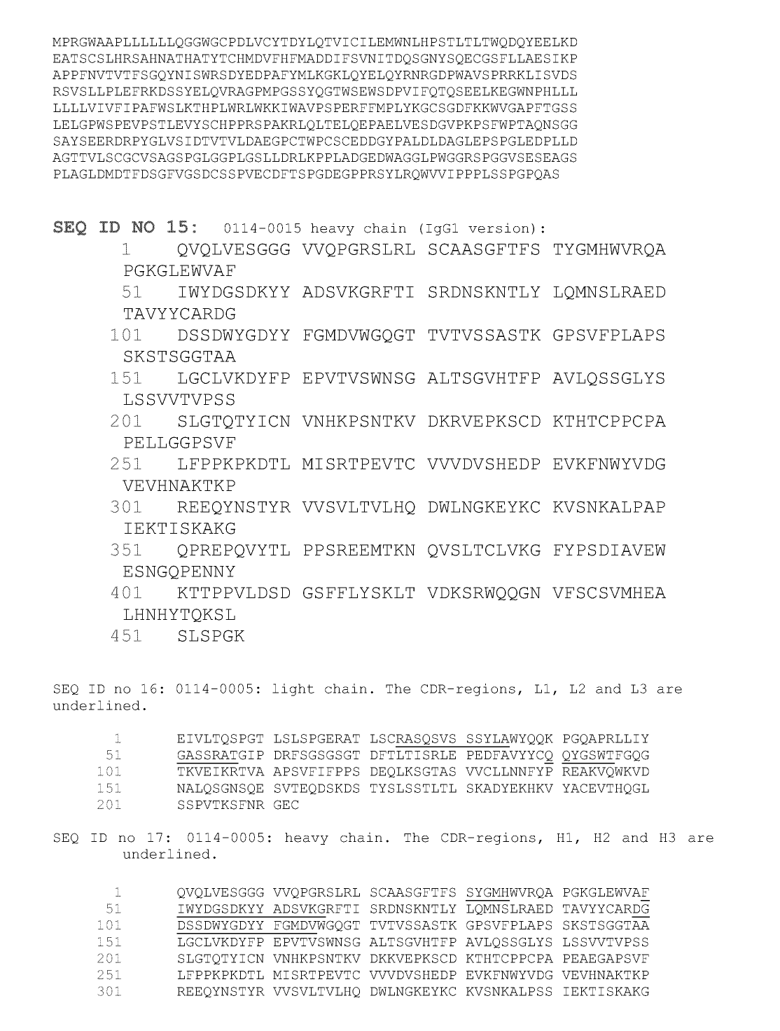

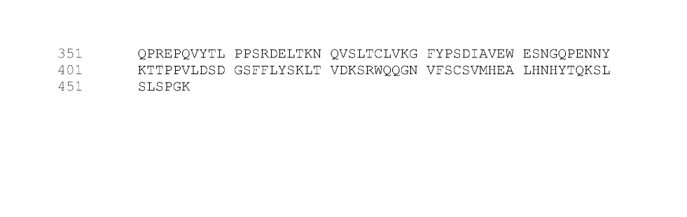

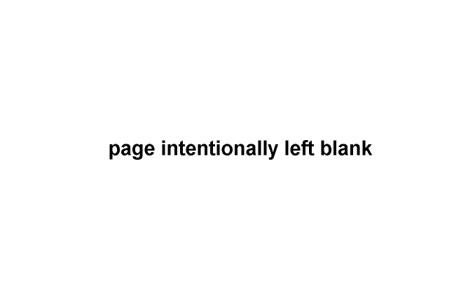

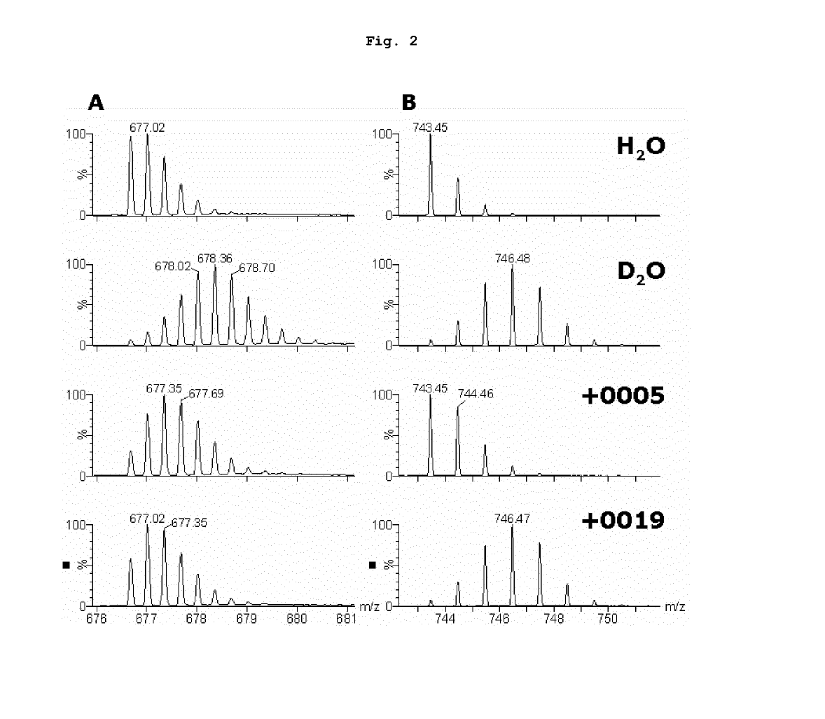

Amide hydrogen/deuterium exchange (HX) was initiated by a 10-fold dilution of hIL-21 in the presence or absence of NNC 0114-0005 or NNC 0114-0019 into the corresponding deuterated buffer (i.e. PBS prepared in D₂O, 96% D₂O final, pH 7.4 (uncorrected value)). All HX reactions were carried out at 20° C. and contained 4 μM hIL-21 in the absence or presence of 2.4 μM mAb thus giving a 1.2 fold molar excess of mAb binding sites. At appropriate time intervals ranging from 10 sec to 10000 sec, 50 μl aliquots of the HX reaction were quenched by 50 μl ice-cold quenching buffer (1.35M TCEP) resulting in a final pH of 2.5 (uncorrected value). Examples of raw data identifying the NNC 0114-0005 and NNC 0114-0019 epitope is shown in FIG. 2.

### Results and Discussion

### Epitope Mapping of NNC 0114-0005

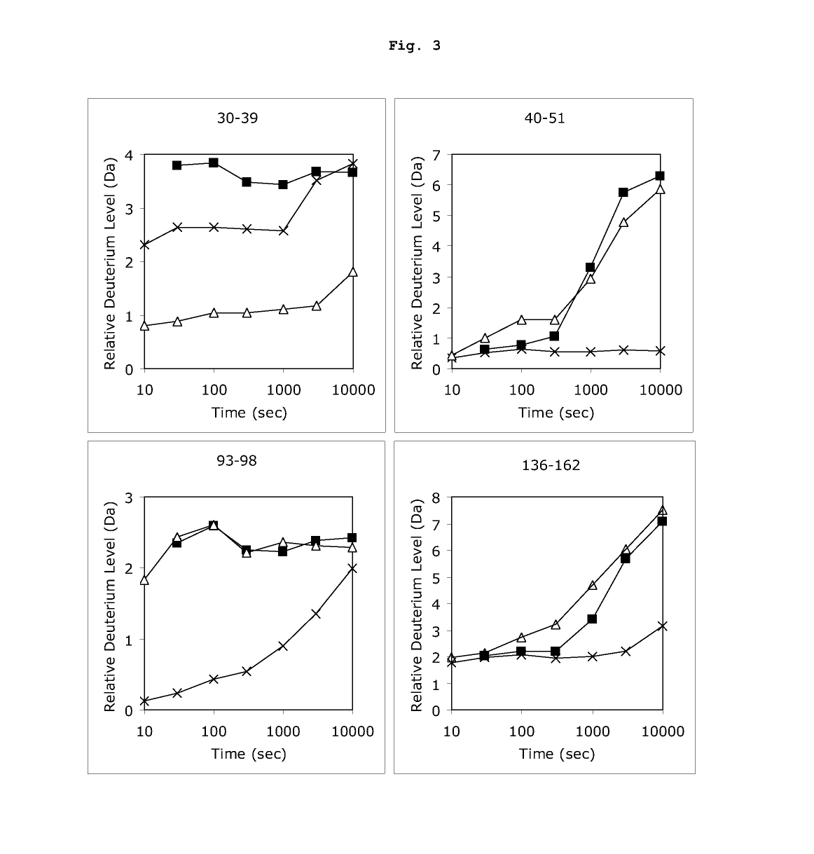

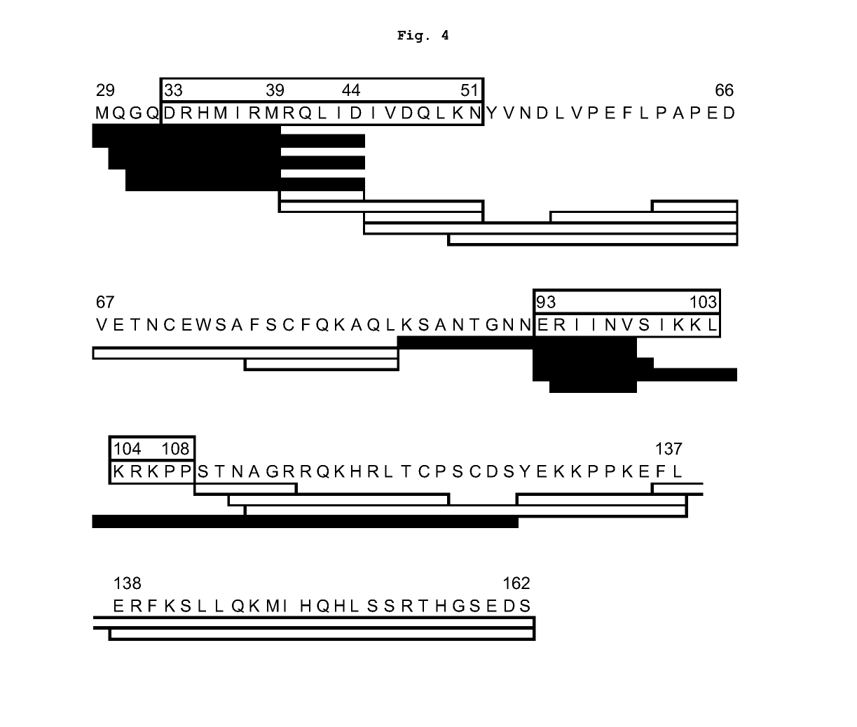

The HX time-course of 26 peptides, covering 100% of the primary sequence of hIL-21 were monitored in the absence or presence of NNC 0114-0005 for 10 to 10000 sec (FIGS. 2, 3, 4). However region 40-51, a central part of helix A, does not contain fast exchanging amide hydrogens (FIG. 3). Thus, no epitope information is gained from this region by HX-MS.

The observed exchange pattern in the early timepoints (<300 sec) in the presence or absence of NNC 0114-0005 can be divided into two different groups: One group of peptides display an exchange pattern that is unaffected by the binding of NNC 0114-0005 in the early timepoints. In contrast, another group of peptides in hIL-21 show protection from exchange upon NNC 0114-0005 binding (FIG. 3). For example at 100 sec exchange with D₂O, approximately 2 amides are protected from exchange in the region E93-V98 upon NNC 0114-0005 binding (FIGS. 2B, 3). The regions displaying protection upon NNC 0114-0005 binding encompass peptides covering residues M29-D44 and K85-S127. However, by comparing the relative amounts of exchange protection within each peptide the epitope for NNC 0114-0005 and the lack of epitope effects in peptides beginning at residue S109 and forward, the epitope can be narrowed to residues D33-D44 and E93-P108. Furthermore, since no epitope information is obtained for region 145-N51 and the epitope for NNC 0114-0005 borders to this region and additional parts of the NNC 0114-0005 epitope could also reside in the 145-N51 region. Although distant in sequence, the D33-D44 and E93-P108 regions are close in the 3D structure of hIL-21.

### Epitope Mapping of NNC 0114-0019

The HX time-course of 26 peptides, covering 100% of the primary sequence of hIL-21 were monitored in the absence or presence of NNC 0114-0019 for 10 to 10000 sec (FIGS. 2, 3, 5). However region R40-N51, a central part of helix A, does not contain fast exchanging amide hydrogens (FIG. 3). Thus, no epitope information is gained from this region by HX-MS.

The observed exchange pattern in the presence or absence of NNC 0114-0019 can be divided into two different groups: One group of peptides display an exchange pattern that is unaffected by the binding of NNC 0114-0019. In contrast, another group of peptides in hIL-21 show protection from exchange upon NNC 0114-0019 binding (FIG. 3). For example at 100 sec exchange with D₂O, approximately 3 amides are protected from exchange in the region Q30-M39 upon NNC 0114-0019 binding (FIG. 3). The regions displaying protection upon NNC 0114-0019 binding encompass peptides covering residues M29-D44 (FIG. 5). By comparing the relative amounts of exchange protection within each peptide the epitope for NNC 0114-0019 can be narrowed to residues Q32-M39, QDRHMIRM.

NNC 0114-0005 Stabilizes the Entire hIL-21 Structure

Apart from epitope effects; the HX time-course of all 26 peptides, covering 100% of the primary sequence of hIL-21 also displayed additional very interesting and surprising features. Binding of NNC 0114-0005 to hIL-21 resulted in a marked reduction of deuterium exchange of hIL-21 in the late time-points of HX exchange, i.e. above 300 sec (see for example R40-N51 and F136-S162 in FIG. 3). Effects observed late in the time course are related to slow exchanging amide hydrogens and thus related to the structural core of the protein. On the contrary, early effects are related to the surface exposed amide hydrogens. Thus, epitope effects appear in the early time points whereas structural stabilization effects will manifest as exchange reduction in late time points (Garcia, Pantazatos and Villareal, Assay and Drug Dev. Tech. 2, 81 (2004); Mandell, Falick and Komives, Proc. Natl. Acad. Sci. USA, 95, 14705 (1998); Andersen and Faber, Int. J. Mass Spec., 302, 139-148 (2011). While minor structural stabilization effects normally can occur locally around a binding site, the effects observed here in hIL-21 upon binding of NNC 0114-0005 to hIL-21 are highly remarkable for several reasons: 1) The R40-N51 and F136-S162 shown in FIG. 3 are only examples. The structural stabilization effects are observed in every part of the entire hIL-21 molecule. Thus, upon binding of NNC 0114-0005 to hIL-21 every region of hIL-21 is stabilized and thus also including all regions very distal to the NNC 0114-0005 epitope. 2) The magnitude of the stabilization effects are unusually large as shown in FIG. 3 with six and four amide hydrogens protected from exchange after 10000 sec in regions R40-N51 and F136-S162, respectively. 3) The effects are only observed upon NNC 0114-0005 binding to hIL-21 and not upon NNC 0114-0019 binding to hIL-21. NNC 0114-0019 binds only in hIL-21 Helix A whereas NNC 0114-0005 binds an overlapping part of helix A as well as a part of hIL-21 helix C. It appears that binding and stabilization of helix C has global effects on structural stabilization of the entire hIL-21 molecule. This structural stabilization of hIL-21 can afford an explanation for the high affinities observed for mAbs binding to this epitope and thus an explanation for the high efficiency of these molecules towards neutralizing the hIL-21 effects.

### Example 8

### Crystal Structure of hIL-21 in Complex with a Fab of Mutated Anti-hIL-21, NNCD 0114-0000-0048

The 3-dimensional structure of hIL-21 in complex with a Fab fragment (NNCD 0114-0000-0048) of a Light-chain residue 54 Ser to Thr mutated form (L:S54T) of the anti hIL-21 human monoclonal antibody NNC 0114-0005 was solved and refined to 1.95 Å resolution using X-ray crystallography. The results demonstrate that the epitope on hIL-21 has an extensive overlap with the binding site for the private hIL-21 receptor chain (IL-21Rα). Thus, by virtue of its high affinity, the anti-hIL-21 mAb efficiently blocks the binding of hIL-21 to IL-21Rα, and, hence, inhibits the biological effects mediated by hIL-21 through its cognate receptor.

### Materials and Methods

hIL-21 (residues 30-162 of SEQ ID NO:1) and anti-IL-21 Fab (NNCD 0114-0000-0048) were mixed with a slight molar excess of hIL-21 and the complex was purified using size exclusion chromatography. The complex was then concentrated to about 15 mg/ml. Crystals were grown with the hanging drop-technique in 25% w/v PEG 3350, 0.1 M Citric Acid, pH 3.5, mixed in a ratio of 1:1.5 (precipitant solution volume:protein solution volume). Total drop size was 3.0 μl. A crystal was prepared for cryo-freezing by transferring 3 μl of a cryo-solution containing 75% of the precipitant solution and 25% glycerol to the drop containing the crystal, and soaking was allowed for about one minute. The crystal was then flash frozen in liquid N₂ and kept at a temperature of 100 K during data collection by a cryogenic N₂ gas stream. Crystallographic data were collected to 1.95 Å resolution at beam-line BL911-3 at MAX-lab, Lund, Sweden. Space group determination, integration and scaling of the data were made by the XDS software package. Cell parameters for the data were determined to be 40.8, 132.8, 53.3 Å, 90°, 106.83° and 90°, respectively, and the space group P2₁. R-sym to 1.95 Å resolution was 5.6% and completeness 98.6%. As the crystal was highly isomorphous with crystal of the complex of the Fab fragment NNC 0114-0009 of the human anti-IL-21 monoclonal antibody NNC 0114-0005 in complex with hIL-21, see Example 1, the 3-dimensional coordinates of this complex were used as input for an initial run of crystallographic rigid-body refinemt using the software program Refmac5 of the CCP4 software package and followed by restrained individual! refinement. The refined model was subjected to computer-graphics inspection of the generated electron density maps, model corrections and building using the Coot software program. Difference density at the site of the mutation was clearly seen and the model corrected accordingly. The procedure was cycled until no further significant improvements could be made to the model. Final R- and R-free for all data were 0.165 and 0.223, respectively, and the model showed a root-mean-square deviation (RMSD) from ideal bond lengths of 0.023 Å (Table 7).

### Results

Light-chain residue 54, Ser to Thr, mutated Anti-IL-21 Fab effectively blocks IL-21Rα binding to site 1 of the hIL-21 molecule, in an almost identical manner as the Fab fragment NNC 0114-0009 of the human anti-IL-21 monoclonal antibody NNC 0114-0005 by binding to the same epitope on hIL-21

Calculation of the areas excluded in pair-wise interactions by the software program AREAIMOL of the CCP4 program suite gave for the hIL-21/anti-hIL-21(L:S54T) Fab molecular complex in the crystal structure 1313 for hIL-21 and 1226 A² for anti-IL-21, respectively. The average area excluded in pair-wise interaction between IL-21 molecule and anti-IL-21 Fab was calculated to be 1270 A².

The direct contacts between the hIL-21 and anti-hIL-21(L:S54T) Fab were identified by running the CONTACT software of the CCP4 program suite using a cut-off distance of 4.0 Å between the anti-IL-21 Fab and the hIL-21 molecules. The results from the hIL-21/anti-IL-21(L:S54T) Fab complex crystal structure are shown in Table 8. The resulting hIL-21 epitope for anti-IL-21(L:S54T) was found to comprise the following residues of hIL-21 (SEQ ID NO: 1): Arg 34, His 35, Ile 37, Arg 38, Gln 41, Asp 44, Ile 45, Gln 48, Asn 51, Tyr 52, Asn 92, Arg 94, Ile 95, Val 98, Ser 99, Lys 101, Lys 102, Arg 105, Arg 105, Pro 107 and Pro 108.

Thus, the anti-IL-21 hIL-21(L:S54T) epitope comprise residues of helix A and C. Additionally, several contact residues (Arg 105 to Pro 108) were identified in the loop segment proceeding helix C. These contact areas agreed very well with what have been determined as the binding site for IL-21Rα on hIL-21 (Example 2).

The anti-IL-21(L:S54T) paratope for hIL-21 included residues Glu 1, Gln 27, Ser 28, Val 29, Ser 30, Ser 32, Tyr 33, Gln 91, Tyr 92, Gly 93, Ser 94 and Trp 95 of the light (L) chain (Table 8), and residues Trp 47, Trp 52, Ser 56, Asp 57, Tyr 59, Tyr 60, Asp 101, Ser 102, Ser 103, Asp 104, Trp 105, Tyr 106, Gly 107, Asp 108, Tyr 109 and Phe 111 of the heavy (H) chain (Table 8).

TABLE 7
Results from the X-ray model refinement to the observed data of the hIL-21/anti-IL-21(L:
S54T) Fab complex by the software program Refmac5 of the CCP4 program software package.

| REMARK | 3 | REFINEMENT. | |
|---|---|---|---|
| REMARK | 3 | PROGRAM: REFMAC 5.5.0109 | |
| REMARK | 3 | AUTHORS: MURSHUDOV, VAGIN, DODSON | |
| REMARK | 3 | | |
| REMARK | 3 | REFINEMENT TARGET: MAXIMUM LIKELIHOOD | |
| REMARK | 3 | | |
| REMARK | 3 | DATA USED IN REFINEMENT. | |
| REMARK | 3 | RESOLUTION RANGE HIGH (ANGSTROMS) | : 1.95 |
| REMARK | 3 | RESOLUTION RANGE LOW (ANGSTROMS) | : 28.20 |
| REMARK | 3 | DATA CUTOFF (SIGMA(F)) | : NONE |
| REMARK | 3 | COMPLETENESS FOR RANGE (%) | : 98.64 |
| REMARK | 3 | NUMBER OF REFLECTIONS | : 37052 |
| REMARK | 3 | | |
| REMARK | 3 | FIT TO DATA USED IN REFINEMENT. | |

| | | | |
|---|---|---|---|
| REMARK | 3 | CROSS-VALIDATION METHOD | : THROUGHOUT |
| REMARK | 3 | FREE R VALUE TEST SET SELECTION | : RANDOM |
| REMARK | 3 | R VALUE (WORKING + TEST SET) | : 0.16805 |
| REMARK | 3 | R VALUE (WORKING SET) | : 0.16507 |
| REMARK | 3 | FREE R VALUE | : 0.22327 |
| REMARK | 3 | FREE R VALUE TEST SET SIZE (%) | : 5.1 |
| REMARK | 3 | FREE R VALUE TEST SET COUNT | : 1981 |
| REMARK | 3 | | |

| | | | |
|---|---|---|---|
| REMARK | 3 | FIT IN THE HIGHEST RESOLUTION BIN. | |
| REMARK | 3 | TOTAL NUMBER OF BINS USED | : 20 |
| REMARK | 3 | BIN RESOLUTION RANGE HIGH | : 1.949 |
| REMARK | 3 | BIN RESOLUTION RANGE LOW | : 2.000 |
| REMARK | 3 | REFLECTION IN BIN (WORKING SET) | : 2479 |
| REMARK | 3 | BIN COMPLETENESS (WORKING + TEST) (%) | : 91.13 |
| REMARK | 3 | BIN R VALUE (WORKING SET) | : 0.223 |
| REMARK | 3 | BIN FREE R VALUE SET COUNT | : 150 |
| REMARK | 3 | BIN FREE R VALUE | : 0.326 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS: 4714 | |
| REMARK | 3 | | |
| REMARK | 3 | B VALUES. | |
| REMARK | 3 | FROM WILSON PLOT (A**2) | : NULL |
| REMARK | 3 | MEAN B VALUE (OVERALL, A**2) | : 26.825 |

| REMARK | 3 | OVERALL ANISOTROPIC B VALUE. | |
|---|---|---|---|
| REMARK | 3 | B11 (A**2): | 21.10 |
| REMARK | 3 | B22 (A**2): | −22.19 |
| REMARK | 3 | B33 (A**2): | 1.09 |
| REMARK | 3 | B12 (A**2): | 0.00 |
| REMARK | 3 | B13 (A**2): | −4.95 |
| REMARK | 3 | B23 (A**2): | 0.00 |
| REMARK | 3 | | |

| | | | |
|---|---|---|---|
| REMARK | 3 | ESTIMATED OVERALL COORDINATE ERROR. | |
| REMARK | 3 | ESU BASED ON R VALUE (A) | : 0.035 |
| REMARK | 3 | ESU BASED ON FREE R VALUE (A) | : 0.034 |
| REMARK | 3 | ESU BASED ON MAXIMUM LIKELIHOOD (A) | : 0.103 |
| REMARK | 3 | ESU FOR B VALUES BASED ON MAXIMUM LIKELIHOOD (A**2) | : 8.810 |
| REMARK | 3 | | |

| | | | |
|---|---|---|---|
| REMARK | 3 | CORRELATION COEFFICIENTS. | |
| REMARK | 3 | CORRELATION COEFFICIENT FO-FC | : 0.963 |
| REMARK | 3 | CORRELATION COEFFICIENT FO-FC FREE | : 0.941 |
| REMARK | 3 | | |

| | | | | | |
|---|---|---|---|---|---|
| REMARK | 3 | RMS DEVIATIONS FROM IDEAL VALUES | COUNT | RMS | WEIGHT |
| REMARK | 3 | BOND LENGTHS REFINED ATOMS (A): | 4398; | 0.023; | 0.022 |
| REMARK | 3 | BOND ANGLES REFINED ATOMS (DEGREES): | 5993; | 1.940; | 1.955 |
| REMARK | 3 | TORSION ANGLES, PERIOD 1 (DEGREES): | 565; | 7.446; | 5.000 |
| REMARK | 3 | TORSION ANGLES, PERIOD 2 (DEGREES): | 187; | 33.270; | 24.064 |
| REMARK | 3 | TORSION ANGLES, PERIOD 3 (DEGREES): | 728; | 15.552; | 15.000 |
| REMARK | 3 | TORSION ANGLES, PERIOD 4 (DEGREES): | 23; | 22.353; | 15.000 |
| REMARK | 3 | CHIRAL-CENTER RESTRAINTS (A**3): | 661; | 0.156; | 0.200 |
| REMARK | 3 | GENERAL PLANES REFINED ATOMS (A): | 3332; | 0.011; | 0.021 |
| REMARK | 3 | | | | |
| REMARK | 3 | ISOTROPIC THERMAL FACTOR RESTRAINTS. | COUNT | RMS | WEIGHT |
| REMARK | 3 | MAIN-CHAIN BOND REFINED ATOMS (A**2): | 2737; | 1.146; | 1.500 |
| REMARK | 3 | MAIN-CHAIN ANGLE REFINED ATOMS (A**2): | 4445; | 1.885; | 2.000 |
| REMARK | 3 | SIDE-CHAIN BOND REFINED ATOMS (A**2): | 1661; | 3.121; | 3.000 |
| REMARK | 3 | SIDE-CHAIN ANGLE REFINED ATOMS (A**2): | 1533; | 4.870; | 4.500 |
| REMARK | 3 | | | | |

| REMARK | 3 | NCS RESTRAINTS STATISTICS | | | | | |
|---|---|---|---|---|---|---|---|
| REMARK | 3 | NUMBER OF NCS GROUPS: NULL | | | | | |
| REMARK | 3 | | | | | | |
| REMARK | 3 | TWIN DETAILS | | | | | |
| REMARK | 3 | NUMBER OF TWIN DOMAINS: NULL | | | | | |
| REMARK | 3 | | | | | | |
| REMARK | 3 | | | | | | |
| REMARK | 3 | TLS DETAILS | | | | | |
| REMARK | 3 | NUMBER OF TLS GROUPS: 3 | | | | | |
| REMARK | 3 | ATOM RECORD CONTAINS SUM OF TLS AND RESIDUAL B FACTORS | | | | | |
| REMARK | 3 | ANISOU RECORD CONTAINS SUM OF TLS AND RESIDUAL U FACTORS | | | | | |
| REMARK | 3 | | | | | | |
| REMARK | 3 | TLS GROUP: 1 | | | | | |
| REMARK | 3 | NUMBER OF COMPONENTS GROUP: 2 | | | | | |
| REMARK | 3 | COMPONENTS | C | SSSEQI | TO | C | SSSEQI |
| REMARK | 3 | RESIDUE RANGE: | L | 1 | | L | 107 |
| REMARK | 3 | RESIDUE RANGE: | H | 1 | | H | 126 |

| | | | | | |
|---|---|---|---|---|---|
| REMARK | 3 | ORIGIN FOR THE GROUP (A): | −4.3834 | 2.4246 | 61.0225 |

| REMARK | 3 | T TENSOR | | | | | |
|---|---|---|---|---|---|---|---|
| REMARK | 3 | T11: | −0.0137 | T22: | 0.1793 | | |
| REMARK | 3 | T33: | 0.0238 | T12: | 0.0154 | | |
| REMARK | 3 | T13: | 0.0266 | T23: | 0.0048 | | |
| REMARK | 3 | L TENSOR | | | | | |
| REMARK | 3 | L11: | 1.4195 | L22: | 2.1152 | | |
| REMARK | 3 | L33: | 1.4674 | L12: | 0.6224 | | |
| REMARK | 3 | L13: | 0.6539 | L23: | 0.0356 | | |
| REMARK | 3 | S TENSOR | | | | | |
| REMARK | 3 | S11: | −0.1027 | S12: | 0.0400 | S13: | 0.0761 |
| REMARK | 3 | S21: | 0.0102 | S22: | 0.0213 | S23: | −0.0125 |
| REMARK | 3 | S31: | −0.1299 | S32: | 0.0295 | S33: | 0.0814 |

| REMARK | 3 | | | | | | |
|---|---|---|---|---|---|---|---|
| REMARK | 3 | TLS GROUP: 2 | | | | | |
| REMARK | 3 | NUMBER OF COMPONENTS GROUP: 2 | | | | | |
| REMARK | 3 | COMPONENTS | C | SSSEQI | TO | C | SSSEQI |
| REMARK | 3 | RESIDUE RANGE: | L | 108 | | L | 250 |
| REMARK | 3 | RESIDUE RANGE: | H | 127 | | H | 250 |

| | | | | | |
|---|---|---|---|---|---|
| REMARK | 3 | ORIGIN FOR THE GROUP (A): | −9.3499 | 28.0027 | 37.7431 |

| REMARK | 3 | T TENSOR | | | | | |
|---|---|---|---|---|---|---|---|
| REMARK | 3 | T11: | 0.0544 | T22: | 0.2010 | | |
| REMARK | 3 | T33: | 0.0746 | T12: | −0.0040 | | |
| REMARK | 3 | T13: | −0.0083 | T23: | −0.0002 | | |
| REMARK | 3 | L TENSOR | | | | | |
| REMARK | 3 | L11: | 1.2074 | L22: | 2.4642 | | |
| REMARK | 3 | L33: | 1.6327 | L12: | −0.2580 | | |
| REMARK | 3 | L13: | 0.7491 | L23: | −0.0910 | | |
| REMARK | 3 | S TENSOR | | | | | |
| REMARK | 3 | S11: | 0.0622 | S12: | 0.1538 | S13: | −0.0260 |
| REMARK | 3 | S21: | −0.2285 | S22: | −0.0478 | S23: | 0.0131 |
| REMARK | 3 | S31: | 0.0989 | S32: | 0.0983 | S33: | −0.0144 |
| REMARK | 3 | | | | | | |

| REMARK | 3 | TLS GROUP: 3 | | | | | |
|---|---|---|---|---|---|---|---|
| REMARK | 3 | NUMBER OF COMPONENTS GROUP: 1 | | | | | |
| REMARK | 3 | COMPONENTS | C | SSSEQI | TO | C | SSSEQI |
| REMARK | 3 | RESIDUE RANGE: | I | 1 | | I | 200 |

| | | | | | |
|---|---|---|---|---|---|
| REMARK | 3 | ORIGIN FOR THE GROUP (A): | −4.6180 | −12.7040 | 86.2350 |

| REMARK | 3 | T TENSOR | | | | | |
|---|---|---|---|---|---|---|---|
| REMARK | 3 | T11: | 0.0951 | T22: | 0.2126 | | |
| REMARK | 3 | T33: | 0.0329 | T12: | −0.0284 | | |
| REMARK | 3 | T13: | 0.0315 | T23: | −0.0135 | | |
| REMARK | 3 | L TENSOR | | | | | |
| REMARK | 3 | L11: | 2.2038 | L22: | 3.6547 | | |
| REMARK | 3 | L33: | 5.2454 | L12: | −0.2189 | | |
| REMARK | 3 | L13: | 0.7775 | L23: | −2.1960 | | |
| REMARK | 3 | S TENSOR | | | | | |
| REMARK | 3 | S11: | 0.0444 | S12: | −0.0533 | S13: | −0.1207 |
| REMARK | 3 | S21: | 0.0846 | S22: | −0.0289 | S23: | 0.0031 |
| REMARK | 3 | S31: | 0.0887 | S32: | 0.0059 | S33: | −0.0155 |
| REMARK | 3 | | | | | | |
| REMARK | 3 | | | | | | |

| REMARK | 3 | BULK SOLVENT MODELLING. | | | |
|---|---|---|---|---|---|
| REMARK | 3 | METHOD USED: MASK | | | |
| REMARK | 3 | PARAMETERS FOR MASK CALCULATION | | | |
| REMARK | 3 | VDW PROBE RADIUS | | | : 1.40 |
| REMARK | 3 | ION PROBE RADIUS | | | : 0.80 |
| REMARK | 3 | SHRINKAGE RADIUS | | | : 0.80 |
| REMARK | 3 | | | | |
| REMARK | 3 | OTHER REFINEMENT REMARKS: | | | |
| REMARK | 3 | HYDROGENS HAVE BEEN ADDED IN THE RIDING POSITIONS | | | |
| REMARK | 3 | U VALUES: RESIDUAL ONLY | | | |
| REMARK | 3 | | | | |
| SSBOND | 1 | CYS L | 89 | CYS L | 23 |

| | | | |
|---|---|---|---|
| LINKR | SG ACYS L 193 | SG ACYS L 133 | SS |
| LINKR | SG BCYS L 193 | SG BCYS L 133 | SS |

| | | | | | |
|---|---|---|---|---|---|
| SSBOND | 2 | CYS H | 96 | CYS H | 22 |
| SSBOND | 3 | CYS H | 209 | CYS H | 153 |
| SSBOND | 4 | CYS I | 71 | CYS I | 122 |

| | | | |
|---|---|---|---|
| LINKR | SG ACYS I 78 | SG ACYS I 125 | SS |
| LINKR | SG BCYS I 78 | SG BCYS I 125 | SS |

| | | | | | | |
|---|---|---|---|---|---|---|
| CISPEP | 1 | SER L | 7 | PRO L | 8 | 0.00 |
| CISPEP | 2 | TYR L | 139 | PRO L | 140 | 0.00 |

| | | | |
|---|---|---|---|
| LINKR | SER H 140 | GLY H 147 | gap |

| | | | | | | |
|---|---|---|---|---|---|---|
| CISPEP | 3 | PHE H | 159 | PRO H | 160 | 0.00 |
| CISPEP | 4 | GLU H | 161 | PRO H | 162 | 0.00 |

| | | | | | |
|---|---|---|---|---|---|
| LINKR | ASN I | 88 | ASN I | 92 | gap |
| LINKR | THR I | 110 | ARG I | 119 | gap |
| LINKR | SER H | 140 | THR H | 148 | gap |
| LINKR | PRO I | 108 | ARG I | 119 | gap |
| LINKR | PHE I | 60 | PRO I | 64 | gap |

| | | | | | | | |
|---|---|---|---|---|---|---|---|
| CRYST1 | 40.850 | 132.840 | 53.320 | 90.00 | 106.83 | 90.00 | P 1 21 1 |

| | | | | |
|---|---|---|---|---|
| SCALE1 | 0.024480 | 0.000000 | 0.007405 | 0.00000 |
| SCALE2 | 0.000000 | 0.007528 | 0.000000 | 0.00000 |
| SCALE3 | 0.000000 | 0.000000 | 0.019594 | 0.00000 |

TABLE 8
hIL-21, chain I, (SEQ ID NO: 1) interactions with the the heavy chain
(chain H) of anti-IL-21 (SEQ ID NO: 17) and light chain (chain L)
mutation Sen 54 to Thr of anti-IL-21 Fab (NNCD 0114-0000-0048). A
distance cut-off of 4.0 Å was used. The contacts were identified
by the CONTACT computer software program of the CCP4 suite. In the
last column “***” indicates a strong possibility for a hydrogen
bond at this contact (distance <3.3 Å) as calculated by CONTACT,
“*” indicates a weak possibility (distance >3.3 Å).
Blank indicates that the program considered there to be no possibility
of a hydrogen bond. Hydrogen-bonds are specific between a donor and
an acceptor, are typically strong, and are easily identifiable.

| hIL-21 | | | aIL-21 (L: S54T) | | | | |
|---|---|---|---|---|---|---|---|
| Res. | Res. # | Atom | Res. | Res. # | Atom | Distance | Possibly |
| Type | and Chain | name | Type | and Chain | name | [Å] | H-bond |
| Arg | 34 I | CB | Tyr | 106 H | CD1 | 3.94 | |
| Arg | 34 I | CD | Tyr | 109 H | CD2 | 3.72 | |
| | | | Tyr | 109 H | CE2 | 3.68 | |
| Arg | 34 I | NE | Tyr | 109 H | CD2 | 3.85 | |
| | | | Tyr | 109 H | CE2 | 3.40 | |
| Arg | 34 I | CZ | Gly | 107 H | O | 3.99 | |
| | | | Tyr | 106 H | O | 3.84 | |
| | | | Tyr | 109 H | CE2 | 3.85 | |
| Arg | 34 I | NH1 | Gly | 107 H | N | 3.79 | * |
| | | | Gly | 107 H | CA | 3.60 | |
| | | | Gly | 107 H | C | 3.39 | |
| | | | Gly | 107 H | O | 3.06 | *** |
| | | | Tyr | 106 H | C | 3.46 | |
| | | | Tyr | 106 H | O | 2.70 | *** |
| His | 35 I | CD2 | Trp | 105 H | NE1 | 3.99 | |
| Ile | 37 I | CD1 | Tyr | 109 H | CZ | 3.87 | |
| | | | Tyr | 109 H | OH | 3.86 | |
| Ile | 37 I | CG2 | Tyr | 109 H | CG | 3.93 | |
| | | | Tyr | 109 H | CD1 | 3.54 | |
| | | | Tyr | 109 H | CE1 | 3.81 | |
| Arg | 38 I | CB | Trp | 105 H | CE2 | 3.63 | |
| | | | Trp | 105 H | CH2 | 3.71 | |
| | | | Trp | 105 H | CZ2 | 3.48 | |
| Arg | 38 I | CG | Trp | 105 H | CE2 | 3.85 | |
| | | | Trp | 105 H | CD2 | 3.90 | |
| | | | Trp | 105 H | O | 3.29 | |
| Arg | 38 I | CD | Trp | 105 H | O | 3.60 | |
| Arg | 38 I | NE | Trp | 105 H | C | 3.62 | |
| | | | Trp | 105 H | CA | 3.80 | |
| | | | Trp | 105 H | O | 2.81 | *** |
| Arg | 38 I | CZ | Asp | 101 H | OD1 | 3.30 | |
| | | | Asp | 104 H | O | 3.87 | |
| | | | Trp | 105 H | O | 3.77 | |
| | | | Asp | 108 H | O | 3.97 | |
| | | | Tyr | 109 H | CA | 3.92 | |
| | | | Tyr | 109 H | CB | 3.56 | |
| Arg | 38 I | NH1 | Asp | 101 H | CG | 3.59 | |
| | | | Asp | 101 H | OD1 | 2.54 | *** |
| | | | Tyr | 109 H | CA | 3.66 | |
| | | | Tyr | 109 H | CB | 3.45 | |
| Arg | 38 I | NH2 | Asp | 101 H | OD1 | 3.26 | *** |
| | | | Ser | 102 H | O | 3.33 | * |
| | | | Asp | 104 H | C | 3.93 | |
| | | | Asp | 104 H | O | 2.91 | *** |
| | | | Trp | 105 H | O | 3.86 | * |
| | | | Gly | 107 H | O | 3.99 | * |
| | | | Asp | 108 H | C | 3.86 | |
| | | | Asp | 108 H | O | 3.00 | *** |
| | | | Tyr | 109 H | CA | 3.85 | |
| | | | Tyr | 109 H | CB | 3.85 | |
| Gln | 41 I | CG | Phe | 111 H | CE1 | 3.74 | |
| | | | Phe | 111 H | CZ | 3.66 | |
| Gln | 41 I | CD | Phe | 111 H | CE1 | 3.47 | |
| | | | Phe | 111 H | CZ | 3.91 | |
| | | | Tyr | 109 H | O | 3.72 | |
| Gln | 41 I | OE1 | Phe | 111 H | CE1 | 3.85 | |
| Gln | 41 I | NE2 | Phe | 111 H | CD1 | 3.93 | |
| | | | Phe | 111 H | CE1 | 3.62 | |
| | | | Tyr | 109 H | CB | 3.77 | |
| | | | Tyr | 109 H | C | 3.71 | |
| | | | Tyr | 109 H | O | 2.63 | *** |
| Asp | 44 I | CA | Ser | 30 L | OG | 3.57 | |
| Asp | 44 I | CB | Ser | 32 L | CB | 3.52 | |
| | | | Ser | 30 L | OG | 3.23 | |
| | | | Ser | 32 L | OG | 3.43 | |
| Asp | 44 I | CG | Ser | 32 L | CB | 3.83 | |
| | | | Ser | 30 L | OG | 3.49 | |
| | | | Ser | 32 L | OG | 3.51 | |
| Asp | 44 I | OD1 | Ser | 30 L | OG | 3.94 | * |
| Asp | 44 I | OD2 | Ser | 32 L | CB | 3.26 | |
| | | | Ser | 30 L | OG | 3.90 | * |
| | | | Ser | 32 L | OG | 2.86 | *** |
| | | | Ser | 32 L | N | 3.17 | *** |
| | | | Ser | 32 L | CA | 3.69 | |
| Asp | 44 I | C | Ser | 30 L | OG | 3.59 | |
| Asp | 44 I | O | Tyr | 33 L | CE2 | 3.82 | |
| | | | Ser | 30 L | CB | 3.57 | |
| | | | Ser | 30 L | OG | 2.93 | *** |
| Ile | 45 I | CA | Tyr | 33 L | OH | 3.68 | |
| | | | Tyr | 33 L | CE2 | 3.99 | |
| Ile | 45 I | CB | Tyr | 33 L | OH | 3.73 | |
| Ile | 45 I | CG1 | Tyr | 33 L | OH | 3.46 | |
| Ile | 45 I | CG2 | Tyr | 33 L | OH | 3.54 | |
| Gln | 48 I | N | Ser | 30 L | CB | 3.97 | |
| Gln | 48 I | CB | Tyr | 33 L | CZ | 3.78 | |
| | | | Tyr | 33 L | CE2 | 3.79 | |
| | | | Ser | 30 L | CB | 3.91 | |
| Gln | 48 I | CG | Tyr | 33 L | CE1 | 3.73 | |
| | | | Tyr | 33 L | CD1 | 4.00 | |
| | | | Tyr | 33 L | CZ | 3.84 | |
| Gln | 48 I | CD | Gln | 91 L | NE2 | 3.68 | |
| | | | Ser | 30 L | N | 3.96 | |
| Gln | 48 I | OE1 | Tyr | 33 L | CD1 | 3.90 | |
| | | | Gln | 91 L | NE2 | 3.38 | * |
| | | | Tyr | 33 L | CB | 3.92 | |
| | | | Tyr | 33 L | CG | 3.78 | |
| | | | Ser | 30 L | N | 3.04 | *** |
| | | | Ser | 30 L | CA | 3.97 | |
| | | | Ser | 30 L | CB | 3.71 | |
| | | | Val | 29 L | CA | 3.64 | |
| | | | Val | 29 L | C | 3.82 | |
| Gln | 48 I | NE2 | Gln | 91 L | NE2 | 3.77 | * |
| | | | Ser | 28 L | O | 3.29 | *** |
| Asn | 51 I | CG | Ser | 28 L | CB | 3.89 | |
| | | | Ser | 28 L | O | 3.97 | |
| Asn | 51 I | OD1 | Ser | 28 L | CB | 3.65 | |
| | | | Ser | 28 L | OG | 3.87 | * |
| Asn | 51 I | ND2 | Ser | 28 L | CB | 3.91 | |
| | | | Ser | 28 L | C | 3.97 | |
| | | | Ser | 28 L | O | 3.43 | * |
| | | | Ser | 28 L | OG | 3.84 | * |
| Tyr | 52 I | CE1 | Gln | 27 L | CD | 3.94 | |
| | | | Gln | 27 L | OE1 | 3.50 | |
| Tyr | 52 I | CZ | Gln | 27 L | OE1 | 3.53 | |
| Tyr | 52 I | OH | Gln | 27 L | CD | 3.72 | |
| | | | Gln | 27 L | OE1 | 2.66 | *** |
| | | | Gln | 91 L | NE2 | 3.95 | * |
| Asn | 92 I | CG | Glu | 1 L | N | 3.74 | |
| Asn | 92 I | OD1 | Glu | 1 L | N | 2.84 | *** |
| | | | Glu | 1 L | CA | 3.67 | |
| | | | Gln | 27 L | OE1 | 3.91 | * |
| | | | Gln | 27 L | NE2 | 3.61 | * |
| Asn | 92 I | ND2 | Gln | 27 L | NE2 | 3.63 | * |
| Arg | 94 I | CG | Tyr | 59 H | CD1 | 3.91 | |
| | | | Gly | 93 L | O | 3.34 | |
| Arg | 94 I | CD | Tyr | 59 H | CB | 3.86 | |
| | | | Trp | 47 H | CZ3 | 3.86 | |
| | | | Trp | 47 H | CH2 | 3.59 | |
| | | | Gly | 93 L | O | 3.71 | |
| Arg | 94 I | NE | Ser | 94 L | O | 3.21 | *** |
| | | | Trp | 47 H | CZ3 | 3.46 | |
| | | | Trp | 47 H | CH2 | 3.73 | |
| | | | Gly | 93 L | O | 3.55 | * |
| | | | Ser | 94 L | C | 3.83 | |
| Arg | 94 I | CZ | Ser | 94 L | O | 3.43 | |
| | | | Trp | 47 H | CZ3 | 3.34 | |
| Arg | 94 I | NH1 | Tyr | 60 H | O | 3.57 | * |
| | | | Trp | 47 H | CZ3 | 3.56 | |
| Arg | 94 I | NH2 | Ser | 94 L | O | 2.77 | *** |
| | | | Trp | 47 H | CZ3 | 3.76 | |
| | | | Ser | 94 L | C | 3.80 | |
| Ile | 95 I | N | Gly | 93 L | O | 3.82 | * |
| Ile | 95 I | CA | Gly | 93 L | C | 3.81 | |
| | | | Gly | 93 L | O | 3.79 | |
| Ile | 95 I | CB | Gly | 93 L | C | 3.76 | |
| | | | Ser | 94 L | N | 3.53 | |
| Ile | 95 I | CG1 | Gly | 93 L | CA | 3.90 | |
| | | | Tyr | 33 L | CE1 | 3.98 | |
| | | | Gly | 93 L | C | 3.86 | |
| | | | Tyr | 92 L | O | 3.68 | |
| | | | Ser | 94 L | N | 3.65 | |
| Ile | 95 I | CD1 | Tyr | 33 L | CE1 | 3.90 | |
| | | | Tyr | 33 L | CD1 | 3.87 | |
| | | | Gln | 91 L | CD | 3.48 | |
| | | | Gln | 91 L | OE1 | 3.74 | |
| | | | Gln | 91 L | NE2 | 3.34 | |
| | | | Tyr | 92 L | O | 3.41 | |
| | | | Ser | 94 L | N | 3.98 | |
| Val | 98 I | CG2 | Tyr | 59 H | CZ | 3.61 | |
| | | | Tyr | 59 H | OH | 3.88 | |
| | | | Tyr | 59 H | CE2 | 3.75 | |
| | | | Tyr | 59 H | CE1 | 3.98 | |
| | | | Trp | 95 L | CH2 | 3.84 | |
| Val | 98 I | O | Trp | 52 H | CH2 | 3.90 | |
| Ser | 99 I | CA | Tyr | 33 L | OH | 3.95 | |
| Ser | 99 I | CB | Tyr | 33 L | OH | 3.33 | |
| Ser | 99 I | OG | Tyr | 33 L | CE1 | 3.45 | |
| | | | Tyr | 33 L | OH | 2.67 | *** |
| | | | Tyr | 33 L | CZ | 3.46 | |
| Lys | 101 I | CB | Trp | 52 H | CH2 | 3.81 | |
| | | | Trp | 52 H | CZ2 | 3.99 | |
| Lys | 101 I | CG | Trp | 52 H | CH2 | 3.86 | |
| | | | Trp | 52 H | CZ2 | 3.55 | |
| Lys | 101 I | CD | Asp | 57 H | OD2 | 3.52 | |
| | | | Trp | 52 H | CE2 | 3.69 | |
| | | | Trp | 52 H | CD2 | 3.96 | |
| | | | Trp | 52 H | CH2 | 3.84 | |
| | | | Trp | 52 H | CZ2 | 3.65 | |
| Lys | 101 I | CE | Ser | 56 H | OG | 3.39 | |
| | | | Asp | 57 H | OD2 | 3.44 | |
| | | | Trp | 52 H | CG | 3.84 | |
| | | | Trp | 52 H | CD1 | 3.71 | |
| | | | Trp | 52 H | NE1 | 3.47 | |
| | | | Trp | 52 H | CE2 | 3.46 | |
| | | | Trp | 52 H | CD2 | 3.69 | |
| | | | Trp | 52 H | CZ2 | 3.96 | |
| Lys | 101 I | NZ | Ser | 56 H | CB | 3.72 | |
| | | | Ser | 56 H | OG | 2.94 | *** |
| | | | Asp | 57 H | OD2 | 3.00 | *** |
| Lys | 102 I | CG | Trp | 52 H | CH2 | 3.87 | |
| Lys | 102 I | CD | Tyr | 92 L | CE2 | 3.81 | |
| Lys | 102 I | CE | Tyr | 92 L | CZ | 3.62 | |
| | | | Tyr | 92 L | OH | 2.98 | |
| | | | Tyr | 92 L | CE2 | 3.39 | |
| Lys | 102 I | NZ | Asp | 101 H | CB | 3.93 | |
| | | | Asp | 101 H | CG | 3.85 | |
| | | | Asp | 101 H | OD2 | 3.01 | *** |
| | | | Tyr | 92 L | CZ | 3.88 | |
| | | | Tyr | 92 L | OH | 2.80 | *** |
| Arg | 105 I | CG | Trp | 105 H | CE3 | 3.87 | |
| Arg | 105 I | CD | Trp | 105 H | CE3 | 3.30 | |
| | | | Trp | 105 H | CZ3 | 3.64 | |
| Arg | 105 I | CZ | Ser | 102 H | O | 3.48 | |
| Arg | 105 I | NH1 | Ser | 103 H | CA | 3.93 | |
| | | | Ser | 103 H | C | 3.45 | |
| | | | Ser | 103 H | O | 3.21 | *** |
| | | | Ser | 102 H | O | 3.06 | *** |
| | | | Asp | 104 H | C | 3.79 | |
| | | | Asp | 104 H | O | 3.87 | * |
| | | | Trp | 105 H | N | 3.78 | * |
| | | | Trp | 105 H | CA | 3.92 | |
| Arg | 105 I | NH2 | Asp | 101 H | CG | 3.41 | |
| | | | Asp | 101 H | OD1 | 2.93 | *** |
| | | | Asp | 101 H | OD2 | 3.32 | * |
| | | | Ser | 102 H | O | 2.98 | *** |
| Pro | 107 I | CA | Trp | 105 H | CZ3 | 4.00 | |
| Pro | 108 I | CB | Tyr | 106 H | OH | 3.75 | |
| | | | Tyr | 106 H | CE2 | 3.81 | |
| Pro | 108 I | CG | Trp | 105 H | NE1 | 3.82 | |
| | | | Tyr | 106 H | OH | 3.73 | |
| Pro | 108 I | CD | Trp | 105 H | NE1 | 3.92 | |
| | | | Trp | 105 H | CE2 | 3.59 | |
| | | | Trp | 105 H | CD2 | 3.71 | |
| | | | Trp | 105 H | CZ2 | 3.91 | |

### Example 9

### Crystal Structure of hIL-21 in Complex with a Fab of Mutated Anti-hIL-21, NNCD 0114-0000-0050

The 3-dimensional structure of hIL-21 in complex with a Fab fragment (NNCD 0114-0000-0050) of a Light-chain residue 57 Thr to Ser mutated form (L:T57S) of the alL-21 human monoclonal antibody NNC 0114-0005 was solved and refined to 1.77 Å resolution using X-ray crystallography. The results demonstrate that the epitope on hIL-21 has an extensive overlap with the binding site for the private hIL-21 receptor chain (IL-21Rα). Thus, by virtue of its high affinity, the anti-IL-21 mAb efficiently blocks the binding of hIL-21 to IL-21Rα, and, hence, inhibits the biological effects mediated by hIL-21 through its cognate receptor.

### Materials and Methods

hIL-21 (residues 30-162 of SEQ ID NO:1) and anti-IL-21 Fab (NNCD 0114-0000-0050) were mixed with a slight molar excess of hIL-21 and the complex was purified using size exclusion chromatography. The complex was then concentrated to about 12 mg/ml. Crystals were grown with the hanging drop-technique in 25% w/v PEG 3350, 0.1 M Citric Acid, pH 3.5, mixed in a ratio of 1:2 (precipitant solution volume:protein solution volume). Total drop size was 3.0 μl. A crystal was prepared for cryo-freezing by transferring 3 μl of a cryo-solution containing 75% of the precipitant solution and 25% glycerol to the drop containing the crystal, and soaking was allowed for about one minute. The crystal was then flash frozen in liquid N₂ and kept at a temperature of 100 K during data collection by a cryogenic N₂ gas stream. Crystallographic data were collected to 1.77 Å resolution at beam-line BL911-2 at MAX-lab, Lund, Sweden. Space group determination, integration and scaling of the data were made by the XDS software package. Cell parameters for the data were determined to be 40.9, 133.0, 53.4 Å, 90°, 106.90° and 90°, respectively, and the space group P2₁. R-sym to 1.77 Å resolution was 5.3% and completeness 97.8%. As the crystal was highly isomorphous with the crystal of the complex of the Fab fragment NNC 0114-0009 of the human anti-IL-21 monoclonal antibody NNC 0114-0005 in complex with hIL-21, see Example 1, the 3-dimensional coordinates of this complex were used at input for an initial run of crystallographic rigid-body refinemt using the software program Refmac5 of the CCP4 software package and followed by restrained individual refinement. The refined model was subjected to computer-graphics inspection of the generated electron density maps, model corrections and building using the Coot software program. Difference density at the site of the mutation was clearly seen and the model corrected accordingly. The procedure was cycled until no further significant improvements could be made to the model. Final R- and R-free for all data were 0.180 and 0.230, respectively, and the model showed a root-mean-square deviation (RMSD) from ideal bond lengths of 0.023 Å (Table 9).

### Results

Light-chain residue 57, Thr to Ser, mutated Anti-IL-21 Fab effectively blocks IL-21Rα binding to site 1 of the hIL-21 molecule, in an almost identical manner as the Fab fragment NNC 0114-0009 of the human anti-IL-21 monoclonal antibody NNC 0114-0005 by binding to the same epitope on hIL-21.

Calculation of the areas excluded in pair-wise interactions by the software program AREAIMOL of the CCP4 program suite gave for the hIL-21/anti-IL-21(L:T57S) Fab molecular complex in the crystal structure 1311 for hIL-21 and 1216 A² for anti-IL-21, respectively. The average area excluded in pair-wise interaction between IL-21 molecule and anti-IL-21 Fab was calculated to be 1263 Å².

The direct contacts between the hIL-21 and anti-IL-21(L:T57S) Fab were identified by running the CONTACT software of the CCP4 program suite using a cut-off distance of 4.0 Å between the anti-IL-21 Fab and the hIL-21 molecules. The results from the hIL-21/anti-IL-21(L:T57S) Fab complex crystal structure are shown in Table 10. The resulting hIL-21 epitope for anti-IL-21(L:T57S) was found to comprise the following residues of hIL-21 (SEQ ID NO: 1): Arg 34, Ile 37, Arg 38, Gln 41, Asp 44, Ile 45, Asp 47, Gln 48, Asn 51, Tyr 52, Asn 92, Arg 94, Ile 95, Val 98, Ser 99, Lys 101, Lys 102, Arg 105, Lys 106, Pro 107 and Pro 108.

Thus, the anti-IL-21 hIL-21(L:T57S) epitope comprise residues of helix A and C. Additionally, several contact residues (Arg 105 to Pro 108) were identified in the loop segment proceeding helix C. These contact areas agreed very well with what have been determined as the binding site for IL-21Rα on hIL-21 (Example 2).

The anti-IL-21(L:T57S) paratope for hIL-21 included residues Glu 1, Gln 27, Ser 28, Val 29, Ser 30, Ser 32, Tyr 33, Gln 91, Tyr 92, Gly 93, Ser 94 and Trp 95 of the light (L) chain (Table 10), and residues Trp 47, Trp 52, Ser 56, Asp 57, Tyr 59, Tyr 60, Asp 101, Ser 102, Ser 103, Asp 104, Trp 105, Tyr 106, Gly 107, Asp 108, Tyr 109 and Phe 111 of the heavy (H) chain (Table 9)

TABLE 9
Results from the X-ray model refinement to the observed data of the hIL-21/anti-IL-21(L: T57S)
Fab complex by the software program Refmac5 (3) of the CCP4 program software package.

| REMARK | 3 | REFINEMENT. | |
|---|---|---|---|
| REMARK | 3 | PROGRAM: REFMAC 5.5.0109 | |
| REMARK | 3 | AUTHORS: MURSHUDOV, VAGIN, DODSON | |
| REMARK | 3 | | |
| REMARK | 3 | REFINEMENT TARGET: MAXIMUM LIKELIHOOD | |
| REMARK | 3 | | |
| REMARK | 3 | DATA USED IN REFINEMENT. | |
| REMARK | 3 | RESOLUTION RANGE HIGH (ANGSTROMS) | : 1.78 |
| REMARK | 3 | RESOLUTION RANGE LOW (ANGSTROMS) | : 28.25 |
| REMARK | 3 | DATA CUTOFF (SIGMA(F)) | : NONE |
| REMARK | 3 | COMPLETENESS FOR RANGE (%) | : 98.99 |
| REMARK | 3 | NUMBER OF REFLECTIONS | : 48822 |
| REMARK | 3 | | |
| REMARK | 3 | FIT TO DATA USED IN REFINEMENT. | |

| | | | |
|---|---|---|---|
| REMARK | 3 | CROSS-VALIDATION METHOD | : THROUGHOUT |
| REMARK | 3 | FREE R VALUE TEST SET SELECTION | : RANDOM |
| REMARK | 3 | R VALUE (WORKING + TEST SET) | : 0.18220 |
| REMARK | 3 | R VALUE (WORKING SET) | : 0.17964 |
| REMARK | 3 | FREE R VALUE | : 0.22976 |
| REMARK | 3 | FREE R VALUE TEST SET SIZE (%) | : 5.0 |
| REMARK | 3 | FREE R VALUE TEST SET COUNT | : 2593 |
| REMARK | 3 | | |

| | | | |
|---|---|---|---|
| REMARK | 3 | FIT IN THE HIGHEST RESOLUTION BIN. | |
| REMARK | 3 | TOTAL NUMBER OF BINS USED | : 20 |
| REMARK | 3 | BIN RESOLUTION RANGE HIGH | : 1.782 |
| REMARK | 3 | BIN RESOLUTION RANGE LOW | : 1.828 |
| REMARK | 3 | REFLECTION IN BIN (WORKING SET) | : 3344 |
| REMARK | 3 | BIN COMPLETENESS (WORKING + TEST) (%) | : 92.53 |
| REMARK | 3 | BIN R VALUE (WORKING SET) | : 0.326 |
| REMARK | 3 | BIN FREE R VALUE SET COUNT | : 150 |
| REMARK | 3 | BIN FREE R VALUE | : 0.364 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS: 4683 | |
| REMARK | 3 | | |
| REMARK | 3 | B VALUES. | |
| REMARK | 3 | FROM WILSON PLOT (A**2) | : NULL |
| REMARK | 3 | MEAN B VALUE (OVERALL, A**2) | : 41.772 |

| REMARK | 3 | OVERALL ANISOTROPIC B VALUE. | |
|---|---|---|---|
| REMARK | 3 | B11 (A**2): | 0.31 |
| REMARK | 3 | B22 (A**2): | −0.48 |
| REMARK | 3 | B33 (A**2): | −0.33 |
| REMARK | 3 | B12 (A**2): | 0.00 |
| REMARK | 3 | B13 (A**2): | −0.85 |
| REMARK | 3 | B23 (A**2): | 0.00 |
| REMARK | 3 | | |

| | | | |
|---|---|---|---|
| REMARK | 3 | ESTIMATED OVERALL COORDINATE ERROR. | |
| REMARK | 3 | ESU BASED ON R VALUE (A) | : 0.129 |
| REMARK | 3 | ESU BASED ON FREE R VALUE (A) | : 0.129 |
| REMARK | 3 | ESU BASED ON MAXIMUM LIKELIHOOD (A) | : 0.091 |
| REMARK | 3 | ESU FOR B VALUES BASED ON MAXIMUM LIKELIHOOD (A**2) | : 2.922 |
| REMARK | 3 | | |

| | | | |
|---|---|---|---|
| REMARK | 3 | CORRELATION COEFFICIENTS. | |
| REMARK | 3 | CORRELATION COEFFICIENT FO-FC | : 0.963 |
| REMARK | 3 | CORRELATION COEFFICIENT FO-FC FREE | : 0.944 |
| REMARK | 3 | | |

| | | | | | |
|---|---|---|---|---|---|
| REMARK | 3 | RMS DEVIATIONS FROM IDEAL VALUES | COUNT | RMS | WEIGHT |
| REMARK | 3 | BOND LENGTHS REFINED ATOMS (A): | 4406; | 0.023; | 0.022 |
| REMARK | 3 | BOND ANGLES REFINED ATOMS (DEGREES): | 6008; | 1.910; | 1.955 |
| REMARK | 3 | TORSION ANGLES, PERIOD 1 (DEGREES): | 572; | 6.567; | 5.000 |
| REMARK | 3 | TORSION ANGLES, PERIOD 2 (DEGREES): | 189; | 33.508; | 23.915 |
| REMARK | 3 | TORSION ANGLES, PERIOD 3 (DEGREES): | 736; | 14.859; | 15.000 |
| REMARK | 3 | TORSION ANGLES, PERIOD 4 (DEGREES): | 25; | 18.841; | 15.000 |
| REMARK | 3 | CHIRAL-CENTER RESTRAINTS (A**3): | 660; | 0.147; | 0.200 |
| REMARK | 3 | GENERAL PLANES REFINED ATOMS (A): | 3346; | 0.010; | 0.021 |
| REMARK | 3 | | | | |
| REMARK | 3 | ISOTROPIC THERMAL FACTOR RESTRAINTS. | COUNT | RMS | WEIGHT |
| REMARK | 3 | MAIN-CHAIN BOND REFINED ATOMS (A**2): | 2734; | 1.320; | 1.500 |
| REMARK | 3 | MAIN-CHAIN ANGLE REFINED ATOMS (A**2): | 4443; | 2.288; | 2.000 |
| REMARK | 3 | SIDE-CHAIN BOND REFINED ATOMS (A**2): | 1672; | 3.443; | 3.000 |
| REMARK | 3 | SIDE-CHAIN ANGLE REFINED ATOMS (A**2): | 1544; | 5.354; | 4.500 |
| REMARK | 3 | | | | |

| REMARK | 3 | NCS RESTRAINTS STATISTICS | | | |
|---|---|---|---|---|---|
| REMARK | 3 | NUMBER OF NCS GROUPS: NULL | | | |
| REMARK | 3 | | | | |
| REMARK | 3 | TWIN DETAILS | | | |
| REMARK | 3 | NUMBER OF TWIN DOMAINS: NULL | | | |
| REMARK | 3 | | | | |
| REMARK | 3 | | | | |
| REMARK | 3 | TLS DETAILS | | | |
| REMARK | 3 | NUMBER OF TLS GROUPS: NULL | | | |
| REMARK | 3 | ATOM RECORD CONTAINS SUM OF TLS AND RESIDUAL B FACTORS | | | |
| REMARK | 3 | ANISOU RECORD CONTAINS SUM OF TLS AND RESIDUAL U FACTORS | | | |
| REMARK | 3 | | | | |
| REMARK | 3 | | | | |
| REMARK | 3 | BULK SOLVENT MODELLING. | | | |
| REMARK | 3 | METHOD USED: MASK | | | |
| REMARK | 3 | PARAMETERS FOR MASK CALCULATION | | | |
| REMARK | 3 | VDW PROBE RADIUS | | | : 1.40 |
| REMARK | 3 | ION PROBE RADIUS | | | : 0.80 |
| REMARK | 3 | SHRINKAGE RADIUS | | | : 0.80 |
| REMARK | 3 | | | | |
| REMARK | 3 | OTHER REFINEMENT REMARKS: | | | |
| REMARK | 3 | HYDROGENS HAVE BEEN ADDED IN THE RIDING POSITIONS | | | |
| REMARK | 3 | U VALUES: REFINED INDIVIDUALLY | | | |
| REMARK | 3 | | | | |
| SSBOND | 1 | CYS L | 89 | CYS L | 23 |

| | | | |
|---|---|---|---|
| LINKR | SG ACYS L 193 | SG ACYS L 133 | SS |
| LINKR | SG BCYS L 193 | SG BCYS L 133 | SS |

| | | | | | |
|---|---|---|---|---|---|
| SSBOND | 2 | CYS H | 96 | CYS H | 22 |
| SSBOND | 3 | CYS H | 209 | CYS H | 153 |
| SSBOND | 4 | CYS I | 71 | CYS I | 122 |

| | | | |
|---|---|---|---|
| LINKR | SG ACYS I 78 | SG ACYS I 125 | SS |
| LINKR | SG BCYS I 78 | SG BCYS I 125 | SS |

| | | | | | | |
|---|---|---|---|---|---|---|
| CISPEP | 1 | SER L | 7 | PRO L | 8 | 0.00 |
| CISPEP | 2 | TYR L | 139 | PRO L | 140 | 0.00 |

| | | | |
|---|---|---|---|
| LINKR | SER H 140 | GLY H 147 | gap |

| | | | | | | |
|---|---|---|---|---|---|---|
| CISPEP | 3 | PHE H | 159 | PRO H | 160 | 0.00 |
| CISPEP | 4 | GLU H | 161 | PRO H | 162 | 0.00 |

| | | | | | |
|---|---|---|---|---|---|
| LINKR | GLY H | 203 | GLN H | 205 | gap |
| LINKR | ASN I | 88 | ASN I | 92 | gap |
| LINKR | PRO I | 108 | ARG I | 119 | gap |
| LINKR | SER H | 140 | THR H | 148 | gap |
| LINKR | PHE I | 60 | PRO I | 64 | gap |

| | | | | | | | |
|---|---|---|---|---|---|---|---|
| CRYST1 | 40.930 | 133.020 | 53.390 | 90.00 | 106.90 | 90.00 | P 1 21 1 |

| | | | | |
|---|---|---|---|---|
| SCALE1 | 0.024432 | 0.000000 | 0.007424 | 0.00000 |
| SCALE2 | 0.000000 | 0.007518 | 0.000000 | 0.00000 |
| SCALE3 | 0.000000 | 0.000000 | 0.019576 | 0.00000 |

TABLE 10
hIL-21, chain I, (SEQ ID NO: 1) interactions with the the heavy chain
(chain H) of anti-IL-21 (SEQ ID NO: 17) and light chain (chain L)
mutation Thr 57 to Ser of anti-IL-21 Fab (NNCD 0114-0000-0050). A
distance cut-off of 4.0 Å was used. The contacts were identified
by the CONTACT computer software program of the CCP4 suite. In the
last column “***” indicates a strong possibility for a hydrogen bond
at this contact (distance <3.3 Å) as calculated by CONTACT,
“*” indicates a weak possibility (distance >3.3 Å). Blank indicates
that the program considered there to be no possibility of a hydrogen
bond. Hydrogen-bonds are specific between a donor and an acceptor,
are typically strong, and are easily identifiable.

| hIL-21 | | | aIL-21 (L:T57S) | | | | |
|---|---|---|---|---|---|---|---|
| | Res. # | | | Res. # | | | |
| Res. | and | Atom | Res. | and | Atom | Distance | Possibly |
| Type | Chain | name | Type | Chain | name | [Å] | H-bond |
| Arg | 34 I | CB | Tyr | 106 H | CD1 | 3.78 | |
| Arg | 34 I | NE | Tyr | 109 H | CD2 | 3.94 | |
| | | | Tyr | 109 H | CE2 | 3.57 | |
| Arg | 34 I | CZ | Gly | 107 H | O | 3.89 | |
| | | | Tyr | 106 H | O | 3.80 | |
| | | | Tyr | 109 H | CE2 | 3.79 | |
| Arg | 34 I | NH1 | Gly | 107 H | CA | 3.59 | |
| | | | Gly | 107 H | N | 3.78 | * |
| | | | Gly | 107 H | C | 3.37 | |
| | | | Gly | 107 H | O | 2.91 | *** |
| | | | Tyr | 106 H | C | 3.38 | |
| | | | Tyr | 106 H | O | 2.63 | *** |
| Ile | 37 I | CD1 | Tyr | 109 H | CE1 | 3.88 | |
| | | | Tyr | 109 H | CZ | 3.63 | |
| | | | Tyr | 109 H | OH | 3.75 | |
| Ile | 37 I | CG2 | Tyr | 109 H | CG | 3.90 | |
| | | | Tyr | 109 H | CD1 | 3.44 | |
| | | | Tyr | 109 H | CE1 | 3.77 | |
| Arg | 38 I | CB | Trp | 105 H | CE2 | 3.62 | |
| | | | Trp | 105 H | CH2 | 3.76 | |
| | | | Trp | 105 H | CZ2 | 3.49 | |
| Arg | 38 I | CG | Trp | 105 H | NE1 | 3.98 | |
| | | | Trp | 105 H | CE2 | 3.74 | |
| | | | Trp | 105 H | CD2 | 3.82 | |
| | | | Trp | 105 H | O | 3.43 | |
| Arg | 38 I | CD | Trp | 105 H | O | 3.70 | |
| Arg | 38 I | NE | Trp | 105 H | CA | 3.82 | |
| | | | Trp | 105 H | C | 3.67 | |
| | | | Trp | 105 H | O | 2.86 | *** |
| Arg | 38 I | CZ | Asp | 101 H | OD1 | 3.40 | |
| | | | Trp | 105 H | O | 3.75 | |
| | | | Tyr | 109 H | CB | 3.64 | |
| | | | Tyr | 109 H | CA | 3.98 | |
| Arg | 38 I | NH1 | Asp | 101 H | CG | 3.61 | |
| | | | Asp | 101 H | OD1 | 2.62 | *** |
| | | | Tyr | 109 H | CB | 3.43 | |
| | | | Tyr | 109 H | CA | 3.62 | |
| Arg | 38 I | NH2 | Asp | 101 H | OD1 | 3.34 | * |
| | | | Ser | 102 H | O | 3.35 | * |
| | | | Asp | 104 H | C | 3.96 | |
| | | | Asp | 104 H | O | 3.00 | *** |
| | | | Trp | 105 H | O | 3.77 | * |
| | | | Gly | 107 H | O | 3.98 | * |
| | | | Asp | 108 H | C | 3.99 | |
| | | | Asp | 108 H | O | 3.10 | *** |
| | | | Tyr | 109 H | CB | 3.93 | |
| | | | Tyr | 109 H | CA | 3.91 | |
| Gln | 41 I | CD | Phe | 111 H | CE1 | 3.64 | |
| | | | Tyr | 109 H | O | 3.78 | |
| Gln | 41 I | OE1 | Phe | 111 H | CE1 | 3.60 | |
| Gln | 41 I | NE2 | Phe | 111 H | CE1 | 3.78 | |
| | | | Tyr | 109 H | CB | 3.64 | |
| | | | Tyr | 109 H | CD1 | 3.79 | |
| | | | Tyr | 109 H | C | 3.71 | |
| | | | Tyr | 109 H | O | 2.62 | *** |
| Asp | 44 I | CA | Ser | 30 L | OG | 3.38 | |
| Asp | 44 I | CB | Ser | 32 L | CB | 3.44 | |
| | | | Ser | 30 L | OG | 3.34 | |
| | | | Ser | 32 L | OG | 3.31 | |
| Asp | 44 I | CG | Ser | 32 L | CB | 3.75 | |
| | | | Ser | 30 L | OG | 3.65 | |
| | | | Ser | 32 L | OG | 3.31 | |
| | | | Ser | 32 L | N | 3.94 | |
| Asp | 44 I | OD1 | Ser | 30 L | OG | 3.92 | * |
| Asp | 44 I | OD2 | Ser | 32 L | CB | 3.16 | |
| | | | Ser | 32 L | OG | 2.60 | *** |
| | | | Ser | 32 L | N | 3.05 | *** |
| | | | Ser | 32 L | CA | 3.54 | |
| Asp | 44 I | C | Ser | 30 L | OG | 3.44 | |
| Asp | 44 I | O | Tyr | 33 L | CE2 | 3.86 | |
| | | | Ser | 30 L | CB | 3.43 | |
| | | | Ser | 30 L | OG | 2.78 | *** |
| Ile | 45 I | CA | Tyr | 33 L | OH | 3.71 | |
| Ile | 45 I | CB | Tyr | 33 L | OH | 3.81 | |
| Ile | 45 I | CG1 | Tyr | 33 L | OH | 3.45 | |
| Ile | 45 I | CG2 | Tyr | 33 L | OH | 3.71 | |
| Asp | 47 I | CB | Ser | 30 L | OG | 3.98 | |
| Gln | 48 I | CB | Tyr | 33 L | OH | 3.95 | |
| | | | Tyr | 33 L | CZ | 3.72 | |
| | | | Tyr | 33 L | CE2 | 3.68 | |
| | | | Ser | 30 L | CB | 3.81 | |
| Gln | 48 I | CG | Tyr | 33 L | CE1 | 3.74 | |
| | | | Tyr | 33 L | CZ | 3.78 | |
| Gln | 48 I | CD | Gln | 91 L | NE2 | 3.67 | |
| | | | Ser | 30 L | N | 3.85 | |
| Gln | 48 I | OE1 | Tyr | 33 L | CD1 | 3.90 | |
| | | | Gln | 91 L | NE2 | 3.40 | * |
| | | | Ser | 30 L | N | 2.87 | *** |
| | | | Tyr | 33 L | CB | 3.88 | |
| | | | Tyr | 33 L | CG | 3.70 | |
| | | | Ser | 30 L | CA | 3.82 | |
| | | | Ser | 30 L | CB | 3.50 | |
| | | | Val | 29 L | CA | 3.52 | |
| | | | Val | 29 L | C | 3.66 | |
| Gln | 48 I | NE2 | Gln | 91 L | NE2 | 3.76 | * |
| | | | Ser | 28 L | O | 3.26 | *** |
| Asn | 51 I | ND2 | Ser | 28 L | CB | 3.81 | |
| | | | Ser | 28 L | C | 3.80 | |
| | | | Ser | 28 L | O | 3.27 | *** |
| | | | Ser | 28 L | OG | 3.92 | * |
| Tyr | 52 I | CE1 | Gln | 27 L | OE1 | 3.23 | |
| Tyr | 52 I | CZ | Gln | 27 L | OE1 | 3.31 | |
| Tyr | 52 I | OH | Gln | 27 L | CD | 3.75 | |
| | | | Gln | 27 L | OE1 | 2.53 | *** |
| Asn | 92 I | CG | Glu | 1 L | N | 3.62 | |
| Asn | 92 I | OD1 | Glu | 1 L | N | 2.67 | *** |
| | | | Glu | 1 L | CA | 3.60 | |
| | | | Gln | 27 L | OE1 | 3.71 | * |
| | | | Gln | 27 L | NE2 | 3.54 | * |
| Arg | 94 I | CG | Tyr | 59 H | CD1 | 3.81 | |
| | | | Gly | 93 L | O | 3.54 | |
| Arg | 94 I | CD | Tyr | 59 H | CB | 3.88 | |
| | | | Trp | 47 H | CZ3 | 3.85 | |
| | | | Trp | 47 H | CH2 | 3.51 | |
| | | | Gly | 93 L | O | 3.78 | |
| Arg | 94 I | NE | Trp | 47 H | CZ3 | 3.52 | |
| | | | Trp | 47 H | CH2 | 3.72 | |
| | | | Ser | 94 L | O | 3.21 | *** |
| | | | Gly | 93 L | O | 3.56 | * |
| | | | Ser | 94 L | C | 3.85 | |
| Arg | 94 I | CZ | Trp | 47 H | CZ3 | 3.42 | |
| | | | Ser | 94 L | O | 3.39 | |
| Arg | 94 I | NH1 | Tyr | 60 H | O | 3.70 | * |
| | | | Trp | 47 H | CZ3 | 3.62 | |
| Arg | 94 I | NH2 | Trp | 47 H | CZ3 | 3.86 | |
| | | | Ser | 94 L | O | 2.74 | *** |
| | | | Ser | 94 L | C | 3.77 | |
| Ile | 95 I | N | Gly | 93 L | O | 3.81 | * |
| Ile | 95 I | CA | Gly | 93 L | C | 3.81 | |
| | | | Gly | 93 L | O | 3.84 | |
| Ile | 95 I | CB | Gly | 93 L | C | 3.88 | |
| | | | Ser | 94 L | N | 3.65 | |
| Ile | 95 I | CG1 | Tyr | 92 L | O | 3.86 | |
| | | | Ser | 94 L | N | 3.92 | |
| Ile | 95 I | CD1 | Tyr | 92 L | O | 3.34 | |
| | | | Tyr | 33 L | CD1 | 3.86 | |
| | | | Tyr | 33 L | CE1 | 3.80 | |
| | | | Gln | 91 L | CD | 3.57 | |
| | | | Gln | 91 L | NE2 | 3.35 | |
| | | | Gln | 91 L | OE1 | 3.81 | |
| Val | 98 I | CG2 | Tyr | 59 H | OH | 3.94 | |
| | | | Tyr | 59 H | CE1 | 3.86 | |
| | | | Tyr | 59 H | CZ | 3.65 | |
| | | | Tyr | 59 H | CE2 | 3.87 | |
| | | | Trp | 95 L | CZ3 | 3.93 | |
| | | | Trp | 95 L | CH2 | 3.94 | |
| Val | 98 I | O | Trp | 52 H | CH2 | 3.94 | |
| Ser | 99 I | CB | Tyr | 33 L | OH | 3.48 | |
| Ser | 99 I | OG | Tyr | 33 L | OH | 2.71 | *** |
| | | | Tyr | 33 L | CE1 | 3.64 | |
| | | | Tyr | 33 L | CZ | 3.54 | |
| Lys | 101 I | CB | Trp | 52 H | CH2 | 3.70 | |
| | | | Trp | 52 H | CZ2 | 3.89 | |
| Lys | 101 I | CG | Trp | 52 H | CE2 | 3.91 | |
| | | | Trp | 52 H | CH2 | 3.73 | |
| | | | Trp | 52 H | CZ2 | 3.43 | |
| Lys | 101 I | CD | Asp | 57 H | OD2 | 3.62 | |
| | | | Trp | 52 H | CE2 | 3.68 | |
| | | | Trp | 52 H | CD2 | 3.93 | |
| | | | Trp | 52 H | CH2 | 3.81 | |
| | | | Trp | 52 H | CZ2 | 3.65 | |
| Lys | 101 I | CE | Ser | 56 H | OG | 3.58 | |
| | | | Asp | 57 H | OD2 | 3.46 | |
| | | | Trp | 52 H | CG | 3.77 | |
| | | | Trp | 52 H | CD1 | 3.62 | |
| | | | Trp | 52 H | NE1 | 3.45 | |
| | | | Trp | 52 H | CE2 | 3.36 | |
| | | | Trp | 52 H | CD2 | 3.55 | |
| | | | Trp | 52 H | CZ2 | 3.86 | |
| Lys | 101 I | NZ | Ser | 56 H | OG | 3.25 | *** |
| | | | Asp | 57 H | OD2 | 3.38 | * |
| Lys | 102 I | CG | Trp | 52 H | CH2 | 3.95 | |
| Lys | 102 I | CD | Asp | 101 H | OD2 | 3.63 | |
| Lys | 102 I | CE | Asp | 101 H | CG | 3.97 | |
| | | | Asp | 101 H | OD2 | 2.95 | |
| | | | Tyr | 92 L | CZ | 3.90 | |
| | | | Tyr | 92 L | OH | 3.12 | |
| | | | Tyr | 92 L | CE2 | 3.83 | |
| Lys | 102 I | NZ | Asp | 101 H | OD2 | 3.81 | * |
| | | | Tyr | 92 L | CZ | 3.81 | |
| | | | Tyr | 92 L | OH | 2.73 | *** |
| Arg | 105 I | CG | Trp | 105 H | CE3 | 3.93 | |
| Arg | 105 I | CD | Trp | 105 H | CE3 | 3.45 | |
| | | | Trp | 105 H | CZ3 | 3.74 | |
| Arg | 105 I | CZ | Asp | 101 H | OD1 | 3.98 | |
| | | | Ser | 102 H | O | 3.40 | |
| Arg | 105 I | NH1 | Ser | 103 H | CA | 3.95 | |
| | | | Ser | 103 H | C | 3.50 | |
| | | | Ser | 103 H | O | 3.27 | *** |
| | | | Ser | 102 H | O | 3.12 | *** |
| | | | Asp | 104 H | C | 3.85 | |
| | | | Trp | 105 H | N | 3.75 | * |
| | | | Trp | 105 H | CA | 3.97 | |
| Arg | 105 I | NH2 | Ser | 102 H | C | 3.97 | |
| | | | Asp | 101 H | CG | 3.34 | |
| | | | Asp | 101 H | OD1 | 2.88 | *** |
| | | | Asp | 101 H | OD2 | 3.19 | *** |
| | | | Ser | 102 H | O | 2.85 | *** |
| Lys | 106 I | O | Trp | 105 H | CE3 | 3.91 | |
| Pro | 107 I | CA | Trp | 105 H | CE3 | 3.93 | |
| | | | Trp | 105 H | CZ3 | 3.91 | |
| Pro | 108 I | CB | Tyr | 106 H | OH | 3.81 | |
| | | | Tyr | 106 H | CE2 | 3.78 | |
| Pro | 108 I | CG | Trp | 105 H | CD1 | 3.82 | |
| | | | Trp | 105 H | NE1 | 3.71 | |
| | | | Tyr | 106 H | OH | 3.51 | |
| Pro | 108 I | CD | Trp | 105 H | NE1 | 3.84 | |
| | | | Trp | 105 H | CE2 | 3.60 | |
| | | | Trp | 105 H | CD2 | 3.71 | |
| | | | Trp | 105 H | CZ2 | 3.96 | |

### Example 10

### Crystal Structure of hIL-21 in Complex with a Fab of Mutated Anti-IL-21, NNCD 0114-0000-0051

The 3-dimensional structure of hIL-21 in complex with a Fab fragment (NNCD 0114-0000-0051) of a Heavy-chain residue 31 Ser to Ala mutated form (H:S31A) of the anti-IL-21 human monoclonal antibody NNC 0114-0005 was solved and refined to 1.88 Å resolution using X-ray crystallography. The results demonstrate that the epitope on hIL-21 has an extensive overlap with the binding site for the private hIL-21 receptor chain (IL-21Rα). Thus, by virtue of its high affinity, the anti-IL-21 mAb efficiently blocks the binding of hIL-21 to IL-21Rα, and, hence, inhibits the biological effects mediated by hIL-21 through its cognate receptor.

### Materials and Methods

hIL-21 (residues 30-162 of SEQ ID NO:1) and anti-IL-21 Fab (NNCD 0114-0000-0051) were mixed with a slight molar excess of hIL-21 and the complex was purified using size exclusion chromatography. The complex was then concentrated to about 16 mg/ml. Crystals were grown with the hanging drop-technique in 25% w/v PEG 3350, 0.1 M Citric Acid pH 3.5 mixed in a ratio of 1:2 (precipitant solution volume:protein solution volume). Total drop size was 3.0 μl. A crystal was prepared for cryo-freezing by transferring 3 μl of a cryo-solution containing 75% of the precipitant solution and 25% glycerol to the drop containing the crystal, and soaking was allowed for about one minute. The crystal was then flash frozen in liquid N₂ and kept at a temperature of 100 K during data collection by a cryogenic N₂ gas stream. Crystallographic data were collected to 1.88 Å resolution at beam-line BL911-3 at MAX-lab, Lund, Sweden. Space group determination, integration and scaling of the data were made by the XDS software package. Cell parameters for the data were determined to be 41.0, 133.1, 53.4 Å, 90°, 107.00° and 90°, respectively, and the space group P2₁. R-sym to 1.88 Å resolution was 6.2% and completeness 96.2%. As the crystal was highly isomorphous with the complex of the crystal of the complex of the Fab fragment NNC 0114-0009 of the human anti-IL-21 monoclonal antibody NNC 0114-0005 in complex with hIL-21, see Example 1, the 3-dimensional coordinates of this complex were used at input for an initial run of crystallographic rigid-body refinement using the software program Refmac5 of the CCP4 software package and followed by restrained individual refinement. The refined model was subjected to computer-graphics inspection of the generated electron density maps, model corrections and building using the Coot software program. Difference density at the site of the mutation was clearly seen and the model corrected accordingly. The procedure was cycled until no further significant improvements could be made to the model. Final R- and R-free for all data were 0.169 and 0.222, respectively, and the model showed a root-mean-square deviation (RMSD) from ideal bond lengths of 0.022 Å (Table 11).

### Results

Heavy-chain residue 31, Ser to Ala, mutated Anti-IL-21 Fab effectively blocks IL-21Rα binding to site 1 of the hIL-21 molecule, in an almost identical manner as Anti-IL-21 Fab. fragment NNC 0114-0009 of the human anti-IL-21 monoclonal antibody NNC 0114-0005 by binding to the same epitope on hIL-21.

Calculation of the areas excluded in pair-wise interactions by the software program AREAIMOL of the CCP4 program suite gave for the hIL-21/anti-IL-21(H:S31A) Fab molecular complex in the crystal structure 1305 for hIL-21 and 1222 Å² for anti-IL-21, respectively. The average area excluded in pair-wise interaction between IL-21 molecule and anti-IL-21 Fab was calculated to be 1264 Å².

The direct contacts between the hIL-21 and anti-IL-21(H:S31A) Fab were identified by running the CONTACT software of the CCP4 program suite (4) using a cut-off distance of 4.0 Å between the anti-IL-21 Fab and the hIL-21 molecules. The results from the hIL-21/anti-IL-21(H:S31A) Fab complex crystal structure are shown in Table 12. The resulting hIL-21 epitope for anti-IL-21(H:S31A) was found to comprise the following residues of hIL-21 (SEQ ID NO: 1): Arg 34, Ile 37, Arg 38, Gln 41, Asp 44, Ile 45, Asp 47, Gln 48, Asn 51, Tyr 52, Asn 92, Arg 94, Ile 95, Asn 97, Val 98, Ser 99, Lys 101, Lys 102, Arg 105, Lys 106, Pro 107 and Pro 108.

Thus, the anti-IL-21 hIL-21(H:S31A) epitope comprise residues of helix A and C. Additionally, several contact residues (Arg 105 to Pro 108) were identified in the loop segment proceeding helix C. These contact areas agreed very well with what have been determined as the binding site for IL-21Rα on hIL-21 (Example 2).

The anti-IL-21(H:S31A) paratope for hIL-21 included residues Glu 1, Gln 27, Ser 28, Val 29, Ser 30, Ser 32, Tyr 33, Gln 91, Tyr 92, Gly 93, Ser 94 and Trp 95 of the light (L) chain (Table 12), and residues Trp 47, Trp 52, Ser 56, Asp 57, Tyr 59, Tyr 60, Asp 99, Asp 101, Ser 102, Ser 103, Asp 104, Trp 105, Tyr 106, Gly 107, Asp 108, Tyr 109 and Phe 111 of the heavy (H) chain (Table 12)

TABLE 11
Results from the X-ray model refinement to the observed data of the hIL-
21/anti-IL-21(H:S31A) Fab complex by the software program Refmac of the CCP4
program software package.

| REMARK | 3 | REFINEMENT. | | |
|---|---|---|---|---|
| REMARK | 3 | PROGRAM : REFMAC 5.5.0109 | | |
| REMARK | 3 | AUTHORS : MURSHUDOV, VAGIN, DODSON | | |
| REMARK | 3 | | | |
| REMARK | 3 | REFINEMENT TARGET: MAXIMUM LIKELIHOOD | | |
| REMARK | 3 | | | |
| REMARK | 3 | DATA USED IN REFINEMENT. | | |
| REMARK | 3 | RESOLUTION RANGE HIGH | (ANGSTROMS) | : 1.88 |
| REMARK | 3 | RESOLUTION RANGE LOW | (ANGSTROMS) | : 29.37 |
| REMARK | 3 | DATA CUTOFF | (SIGMA(F)) | : NONE |
| REMARK | 3 | COMPLETENESS FOR RANGE | (%) | : 96.30 |
| REMARK | 3 | NUMBER OF REFLECTIONS | | : 40833 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.17189 |
| REMARK | 3 | R VALUE | (WORKING SET) | : 0.16920 |
| REMARK | 3 | FREE R VALUE | | : 0.22155 |

| | | | | |
|---|---|---|---|---|
| REMARK | 3 | FREE R VALUE TEST SET SIZE | (%) | : 5.1 |
| REMARK | 3 | FREE R VALUE TEST SET COUNT | | : 2193 |
| REMARK | 3 | | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 1.875 |
| REMARK | 3 | BIN RESOLUTION RANGE LOW | | : 1.924 |
| REMARK | 3 | REFLECTION IN BIN | (WORKING SET) | : 2318 |
| REMARK | 3 | BIN COMPLETENESS | (WORKING + TEST) (%) | : 74.92 |
| REMARK | 3 | BIN R VALUE | (WORKING SET) | : 0.310 |

| | | | |
|---|---|---|---|
| REMARK | 3 | BIN FREE R VALUE SET COUNT | : 135 |
| REMARK | 3 | BIN FREE R VALUE | : 0.399 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | : 4706 |
| REMARK | 3 | | |
| REMARK | 3 | B VALUES. | |

| | | | | |
|---|---|---|---|---|
| REMARK | 3 | FROM WILSON PLOT | (A**2) | : NULL |
| REMARK | 3 | MEAN B VALUE | (OVERALL, A**2) | : 24.084 |

| REMARK | 3 | OVERALL ANISOTROPIC B VALUE. | |
|---|---|---|---|
| REMARK | 3 | B11 (A**2) : | 0.49 |
| REMARK | 3 | B22 (A**2) : | −0.88 |
| REMARK | 3 | B33 (A**2) : | 0.13 |
| REMARK | 3 | B12 (A**2) : | 0.00 |
| REMARK | 3 | B13 (A**2) : | −0.45 |
| REMARK | 3 | B23 (A**2) : | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A): | 0.151 |
| REMARK | 3 | ESU BASED ON FREE R VALUE | (A): | 0.145 |
| REMARK | 3 | ESU BASED ON MAXIMUM LIKELIHOOD | (A): | 0.104 |
| REMARK | 3 | ESU FOR B VALUES BASED ON MAXIMUM LIKELIHOOD | (A**2): | 7.906 |
| REMARK | 3 | | | |

| REMARK | 3 | CORRELATION COEFFICIENTS. | |
|---|---|---|---|
| REMARK | 3 | CORRELATION COEFFICIENT FO-FC | : 0.961 |
| REMARK | 3 | CORRELATION COEFFICIENT FO-FC FREE | : 0.939 |
| REMARK | 3 | | |

| REMARK | 3 | RMS DEVIATIONS FROM IDEAL VALUES | | COUNT | | RMS | | WEIGHT |
|---|---|---|---|---|---|---|---|---|
| REMARK | 3 | BOND LENGTHS REFINED ATOMS | (A): | 4396 | ; | 0.022 | ; | 0.022 |
| REMARK | 3 | BOND ANGLES REFINED ATOMS | (DEGREES): | 5996 | ; | 1.938 | ; | 1.955 |
| REMARK | 3 | TORSION ANGLES, PERIOD 1 | (DEGREES): | 568 | ; | 7.110 | ; | 5.000 |
| REMARK | 3 | TORSION ANGLES, PERIOD 2 | (DEGREES): | 189 | ; | 32.128 | ; | 24.021 |
| REMARK | 3 | TORSION ANGLES, PERIOD 3 | (DEGREES): | 730 | ; | 15.330 | ; | 15.000 |
| REMARK | 3 | TORSION ANGLES, PERIOD 4 | (DEGREES): | 24 | ; | 21.565 | ; | 15.000 |
| REMARK | 3 | CHIRAL-CENTER RESTRAINTS | (A**3): | 660 | ; | 0.151 | ; | 0.200 |
| REMARK | 3 | GENERAL PLANES REFINED ATOMS | (A): | 3341 | ; | 0.010 | ; | 0.021 |
| REMARK | 3 | | | | | | | |
| REMARK | 3 | ISOTROPIC THERMAL FACTOR RESTRAINTS. | | COUNT | | RMS | | WEIGHT |
| REMARK | 3 | MAIN-CHAIN BOND REFINED ATOMS | (A**2): | 2730 | ; | 1.084 | ; | 1.500 |
| REMARK | 3 | MAIN-CHAIN ANGLE REFINED ATOMS | (A**2): | 4437 | ; | 1.847 | ; | 2.000 |
| REMARK | 3 | SIDE-CHAIN BOND REFINED ATOMS | (A**2): | 1666 | ; | 3.042 | ; | 3.000 |
| REMARK | 3 | SIDE-CHAIN ANGLE REFINED ATOMS | (A**2): | 1540 | ; | 4.778 | ; | 4.500 |

| REMARK | 3 | | |
|---|---|---|---|
| REMARK | 3 | NCS RESTRAINTS STATISTICS | |
| REMARK | 3 | NUMBER OF NCS GROUPS : NULL | |
| REMARK | 3 | | |
| REMARK | 3 | TWIN DETAILS | |
| REMARK | 3 | NUMBER OF TWIN DOMAINS : NULL | |
| REMARK | 3 | | |
| REMARK | 3 | | |
| REMARK | 3 | TLS DETAILS | |
| REMARK | 3 | NUMBER OF TLS GROUPS : 3 | |
| REMARK | 3 | ATOM RECORD CONTAINS SUM OF TLS AND RESIDUAL B FACTORS | |
| REMARK | 3 | ANISOU RECORD CONTAINS SUM OF TLS AND RESIDUAL U FACTORS | |
| REMARK | 3 | | |
| REMARK | 3 | TLS GROUP : 1 | |
| REMARK | 3 | NUMBER OF COMPONENTS GROUP : 2 | |
| REMARK | 3 | COMPONENTS C SSSEQI TO C SSSEQI | |
| REMARK | 3 | RESIDUE RANGE : L 1 L 107 | |
| REMARK | 3 | RESIDUE RANGE : H 1 H 126 | |
| REMARK | 3 | ORIGIN FOR THE GROUP (A): −4.6768 2.4150 61.0686 | |
| REMARK | 3 | T TENSOR | |
| REMARK | 3 | T11: 0.0432 T22: 0.0779 | |
| REMARK | 3 | T33: 0.0540 T12: 0.0066 | |
| REMARK | 3 | T13: 0.0249 T23: 0.0104 | |
| REMARK | 3 | L TENSOR | |
| REMARK | 3 | L11: 1.6162 L22: 2.1931 | |
| REMARK | 3 | L33: 1.3176 L12: 0.5381 | |
| REMARK | 3 | L13: 0.6022 L23: 0.0497 | |
| REMARK | 3 | S TENSOR | |
| REMARK | 3 | S11: −0.0962 S12: 0.0484 S13: 0.0935 | |
| REMARK | 3 | S21: 0.0410 S22: −0.0049 S23: −0.0233 | |
| REMARK | 3 | S31: −0.1087 S32: 0.0184 S33: 0.1011 | |
| REMARK | 3 | | |
| REMARK | 3 | TLS GROUP : 2 | |
| REMARK | 3 | NUMBER OF COMPONENTS GROUP : 2 | |
| REMARK | 3 | COMPONENTS C SSSEQI TO C SSSEQI | |
| REMARK | 3 | RESIDUE RANGE : L 108 L 250 | |
| REMARK | 3 | RESIDUE RANGE : H 127 H 250 | |
| REMARK | 3 | ORIGIN FOR THE GROUP (A): −9.4732 28.0431 37.9480 | |
| REMARK | 3 | T TENSOR | |
| REMARK | 3 | T11: 0.1120 T22: 0.1086 | |
| REMARK | 3 | T33: 0.0948 T12: 0.0003 | |
| REMARK | 3 | T13: 0.0060 T23: 0.0023 | |
| REMARK | 3 | L TENSOR | |
| REMARK | 3 | L11: 1.4185 L22: 2.6305 | |
| REMARK | 3 | L33: 1.8409 L12: −0.3782 | |
| REMARK | 3 | L13: 0.9195 L23: −0.4436 | |
| REMARK | 3 | S TENSOR | |
| REMARK | 3 | S11: 0.0781 S12: 0.1649 S13: −0.0440 | |
| REMARK | 3 | S21: −0.2634 S22: −0.0539 S23: −0.0181 | |
| REMARK | 3 | S31: 0.1650 S32: 0.1180 S33: −0.0241 | |
| REMARK | 3 | | |
| REMARK | 3 | TLS GROUP : 3 | |
| REMARK | 3 | NUMBER OF COMPONENTS GROUP : 1 | |
| REMARK | 3 | COMPONENTS C SSSEQI TO C SSSEQI | |
| REMARK | 3 | RESIDUE RANGE : I 1 I 200 | |
| REMARK | 3 | ORIGIN FOR THE GROUP (A): −4.6180 −12.7040 86.2350 | |
| REMARK | 3 | T TENSOR | |
| REMARK | 3 | T11: 0.1542 T22: 0.1173 | |
| REMARK | 3 | T33: 0.0575 T12: −0.0321 | |
| REMARK | 3 | T13: 0.0319 T23: −0.0205 | |
| REMARK | 3 | L TENSOR | |
| REMARK | 3 | L11: 2.2467 L22: 3.5382 | |
| REMARK | 3 | L33: 5.2432 L12: −0.1443 | |
| REMARK | 3 | L13: 0.7369 L23: −2.3610 | |
| REMARK | 3 | S TENSOR | |
| REMARK | 3 | S11: 0.0335 S12: −0.0359 S13: −0.0827 | |
| REMARK | 3 | S21: 0.0969 S22: −0.0429 S23: −0.0744 | |
| REMARK | 3 | S31: 0.0655 S32: 0.0416 S33: 0.0094 | |
| REMARK | 3 | | |
| REMARK | 3 | | |
| REMARK | 3 | BULK SOLVENT MODELLING. | |
| REMARK | 3 | METHOD USED : MASK | |
| REMARK | 3 | PARAMETERS FOR MASK CALCULATION | |
| REMARK | 3 | VDW PROBE RADIUS | : 1.40 |
| REMARK | 3 | ION PROBE RADIUS | : 0.80 |
| REMARK | 3 | SHRINKAGE RADIUS | : 0.80 |
| REMARK | 3 | | |

| | | |
|---|---|---|
| REMARK | 3 | OTHER REFINEMENT REMARKS: |
| REMARK | 3 | U VALUES : RESIDUAL ONLY |
| REMARK | 3 | |
| SSBOND | 1 | CYS L 89 CYS L 23 |

| | | |
|---|---|---|
| LINKR | SG ACYS L 193 SG ACYS L 133 | SS |
| LINKR | SG BCYS L 193 SG BCYS L 133 | SS |

| | | |
|---|---|---|
| SSBOND | 2 | CYS H 96 CYS H 22 |
| SSBOND | 3 | CYS H 209 CYS H 153 |
| SSBOND | 4 | CYS I 71 CYS I 122 |

| | | |
|---|---|---|
| LINKR | SG ACYS I 78 SG ACYS I 125 | SS |
| LINKR | SG BCYS I 78 SG BCYS I 125 | SS |

| | | |
|---|---|---|
| CISPEP | 1 | SER L 7 PRO L 8 0.00 |
| CISPEP | 2 | TYR L 139 PRO L 140 0.00 |

| | | |
|---|---|---|
| LINKR | SER H 140 THR H 148 | gap |

| | | |
|---|---|---|
| CISPEP | 3 | PHE H 159 PRO H 160 0.00 |
| CISPEP | 4 | GLU H 161 PRO H 162 0.00 |

| | | | |
|---|---|---|---|
| LINKR | ASN I 88 | ASN I 92 | gap |
| LINKR | PRO I 108 | ARG I 119 | gap |
| LINKR | PHE I 60 | PRO I 64 | gap |

CRYST1 40.970 133.080 53.370 90.00 107.00 90.00 P 1 21 1

| | | |
|---|---|---|
| SCALE1 | 0.024408 0.000000 0.007461 | 0.00000 |
| SCALE2 | 0.000000 0.007514 0.000000 | 0.00000 |
| SCALE3 | 0.000000 0.000000 0.019593 | 0.00000 |

TABLE 12
hIL-21, chain I, (SEQ ID NO: 1) interactions with the the light chain
(chain L) of anti-IL-21 (SEQ ID NO: 16) and heavy chain (chain H)
mutation Ser 31 to Ala of anti-IL-21 Fab (NNCD 0114-0000-0051).
A distance cut-off of 4.0 Å was used. The contacts were
identified by the CONTACT computer software program of the
CCP4 suite. In the last column “***” indicates a strong possibility
for a hydrogen bond at this contact (distance <3.3 Å) as calculated
by CONTACT, “*” indicates a weak possibility (distance >3.3 Å).
Blank indicates that the program considered there to be no possibility of
a hydrogen bond. Hydrogen-bonds are specific between a donor and an
acceptor, are typically strong, and are easily identifiable.

| hIL-21 | | | aIL-21(H:S31A) | | | | |
|---|---|---|---|---|---|---|---|
| | Res. # | | | Res. # | | | |
| Res. | and | Atom | Res. | and | Atom | Distance | Possibly |
| Type | Chain | name | Type | Chain | name | [Å] | H-bond |
| Arg | 34 I | CB | Tyr | 106 H | CD1 | 3.98 | |
| Arg | 34 I | CD | Tyr | 109 H | CD2 | 3.98 | |
| | | | Tyr | 109 H | CE2 | 3.87 | |
| Arg | 34 I | NE | Tyr | 109 H | CE2 | 3.67 | |
| Arg | 34 I | CZ | Gly | 107 H | O | 3.97 | |
| | | | Tyr | 106 H | O | 3.93 | |
| Arg | 34 I | NH1 | Gly | 107 H | N | 3.91 | * |
| | | | Gly | 107 H | CA | 3.51 | |
| | | | Gly | 107 H | C | 3.24 | |
| | | | Gly | 107 H | O | 2.98 | *** |
| | | | Tyr | 106 H | C | 3.62 | |
| | | | Tyr | 106 H | O | 2.89 | *** |
| Ile | 37 I | CD1 | Tyr | 109 H | CZ | 3.84 | |
| | | | Tyr | 109 H | OH | 3.73 | |
| Ile | 37 I | CG2 | Tyr | 109 H | CG | 3.93 | |
| | | | Tyr | 109 H | CD1 | 3.46 | |
| | | | Tyr | 109 H | CE1 | 3.60 | |
| Arg | 38 I | CB | Trp | 105 H | CE2 | 3.66 | |
| | | | Trp | 105 H | CH2 | 3.86 | |
| | | | Trp | 105 H | CZ2 | 3.57 | |
| Arg | 38 I | CG | Trp | 105 H | CE2 | 3.88 | |
| | | | Trp | 105 H | CD2 | 3.95 | |
| | | | Trp | 105 H | O | 3.32 | |
| Arg | 38 I | CD | Trp | 105 H | O | 3.68 | |
| Arg | 38 I | NE | Trp | 105 H | CA | 3.80 | |
| | | | Trp | 105 H | C | 3.67 | |
| | | | Trp | 105 H | O | 2.92 | *** |
| Arg | 38 I | CZ | Asp | 101 H | OD1 | 3.31 | |
| | | | Asp | 104 H | O | 3.97 | |
| | | | Trp | 105 H | O | 3.89 | |
| | | | Tyr | 109 H | CA | 3.92 | |
| | | | Tyr | 109 H | CB | 3.51 | |
| Arg | 38 I | NH1 | Asp | 101 H | CG | 3.61 | |
| | | | Asp | 101 H | OD1 | 2.51 | *** |
| | | | Tyr | 109 H | CA | 3.70 | |
| | | | Tyr | 109 H | CB | 3.40 | |
| Arg | 38 I | NH2 | Asp | 101 H | OD1 | 3.31 | * |
| | | | Ser | 102 H | O | 3.36 | * |
| | | | Asp | 104 H | O | 3.05 | *** |
| | | | Trp | 105 H | O | 3.98 | * |
| | | | Gly | 107 H | O | 3.93 | * |
| | | | Asp | 108 H | C | 3.83 | |
| | | | Asp | 108 H | O | 3.05 | *** |
| | | | Tyr | 109 H | CA | 3.72 | |
| | | | Tyr | 109 H | CB | 3.69 | |
| Gln | 41 I | CG | Phe | 111 H | CE1 | 3.51 | |
| | | | Phe | 111 H | CZ | 3.49 | |
| Gln | 41 I | CD | Phe | 111 H | CE1 | 3.29 | |
| | | | Phe | 111 H | CZ | 3.85 | |
| Gln | 41 I | OE1 | Phe | 111 H | CE1 | 3.70 | |
| Gln | 41 I | NE2 | Phe | 111 H | CD1 | 3.89 | |
| | | | Phe | 111 H | CE1 | 3.45 | |
| | | | Tyr | 109 H | CB | 3.91 | |
| | | | Tyr | 109 H | O | 3.06 | *** |
| Asp | 44 I | CA | Ser | 30 L | OG | 3.47 | |
| Asp | 44 I | CB | Ser | 32 L | CB | 3.54 | |
| | | | Ser | 30 L | OG | 3.39 | |
| | | | Ser | 32 L | OG | 3.50 | |
| Asp | 44 I | CG | Ser | 32 L | N | 3.98 | |
| | | | Ser | 32 L | CB | 3.83 | |
| | | | Ser | 30 L | OG | 3.69 | |
| | | | Ser | 32 L | OG | 3.49 | |
| Asp | 44 I | OD2 | Ser | 32 L | N | 3.09 | *** |
| | | | Ser | 32 L | CA | 3.65 | |
| | | | Ser | 32 L | CB | 3.20 | |
| | | | Ser | 32 L | OG | 2.74 | *** |
| Asp | 44 I | C | Ser | 30 L | OG | 3.54 | |
| Asp | 44 I | O | Tyr | 33 L | CE2 | 3.76 | |
| | | | Ser | 30 L | CB | 3.42 | |
| | | | Ser | 30 L | OG | 2.86 | *** |
| Ile | 45 I | CA | Tyr | 33 L | OH | 3.60 | |
| | | | Tyr | 33 L | CE2 | 3.96 | |
| Ile | 45 I | CB | Tyr | 33 L | OH | 3.76 | |
| Ile | 45 I | CG1 | Tyr | 33 L | OH | 3.52 | |
| Ile | 45 I | CD1 | Phe | 111 H | CZ | 3.97 | |
| Ile | 45 I | CG2 | Tyr | 33 L | OH | 3.74 | |
| Asp | 47 I | CB | Ser | 30 L | OG | 3.55 | |
| Gln | 48 I | CB | Tyr | 33 L | CZ | 3.74 | |
| | | | Tyr | 33 L | OH | 3.99 | |
| | | | Tyr | 33 L | CE2 | 3.70 | |
| | | | Ser | 30 L | CB | 3.75 | |
| Gln | 48 I | CG | Tyr | 33 L | CZ | 3.78 | |
| | | | Tyr | 33 L | CE1 | 3.78 | |
| Gln | 48 I | CD | Gln | 91 L | NE2 | 3.68 | |
| | | | Ser | 30 L | N | 3.87 | |
| Gln | 48 I | OE1 | Tyr | 33 L | CD1 | 3.89 | |
| | | | Gln | 91 L | NE2 | 3.30 | * |
| | | | Tyr | 33 L | CB | 3.86 | |
| | | | Tyr | 33 L | CG | 3.68 | |
| | | | Ser | 30 L | N | 2.96 | *** |
| | | | Ser | 30 L | CA | 3.87 | |
| | | | Ser | 30 L | CB | 3.59 | |
| | | | Val | 29 L | CA | 3.66 | |
| | | | Val | 29 L | C | 3.79 | |
| Gln | 48 I | NE2 | Gln | 91 L | NE2 | 3.77 | * |
| | | | Ser | 28 L | O | 3.41 | * |
| Asn | 51 I | ND2 | Ser | 28 L | C | 3.94 | |
| | | | Ser | 28 L | O | 3.33 | * |
| Tyr | 52 I | CE1 | Gln | 27 L | OE1 | 3.50 | |
| Tyr | 52 I | CZ | Gln | 27 L | OE1 | 3.55 | |
| Tyr | 52 I | OH | Gln | 27 L | CD | 3.84 | |
| | | | Gln | 27 L | OE1 | 2.65 | *** |
| Asn | 92 I | CG | Glu | 1 L | N | 3.67 | |
| Asn | 92 I | OD1 | Glu | 1 L | N | 2.62 | *** |
| | | | Glu | 1 L | CA | 3.58 | |
| | | | Gln | 27 L | OE1 | 3.75 | * |
| | | | Gln | 27 L | NE2 | 3.93 | * |
| Arg | 94 I | CG | Tyr | 59 H | CD1 | 3.85 | |
| | | | Gly | 93 L | O | 3.40 | |
| Arg | 94 I | CD | Tyr | 59 H | CB | 3.92 | |
| | | | Trp | 47 H | CZ3 | 3.93 | |
| | | | Trp | 47 H | CH2 | 3.58 | |
| | | | Gly | 93 L | O | 3.76 | |
| Arg | 94 I | NE | Trp | 47 H | CZ3 | 3.60 | |
| | | | Trp | 47 H | CH2 | 3.76 | |
| | | | Gly | 93 L | O | 3.56 | * |
| | | | Ser | 94 L | C | 3.87 | |
| | | | Ser | 94 L | O | 3.24 | *** |
| Arg | 94 I | CZ | Trp | 47 H | CZ3 | 3.50 | |
| | | | Ser | 94 L | O | 3.42 | |
| Arg | 94 I | NH1 | Tyr | 60 H | O | 3.65 | * |
| | | | Trp | 47 H | CZ3 | 3.67 | |
| Arg | 94 I | NH2 | Trp | 47 H | CZ3 | 3.93 | |
| | | | Ser | 94 L | C | 3.78 | |
| | | | Ser | 94 L | O | 2.73 | *** |
| Ile | 95 I | N | Gly | 93 L | O | 3.91 | * |
| Ile | 95 I | CA | Gly | 93 L | O | 3.86 | |
| | | | Gly | 93 L | C | 3.85 | |
| Ile | 95 I | CB | Gly | 93 L | C | 3.87 | |
| | | | Ser | 94 L | N | 3.54 | |
| Ile | 95 I | CG1 | Ser | 94 L | N | 3.73 | |
| | | | Tyr | 92 L | O | 3.82 | |
| Ile | 95 I | CD1 | Ser | 94 L | N | 3.95 | |
| | | | Gln | 91 L | CD | 3.57 | |
| | | | Gln | 91 L | NE2 | 3.38 | |
| | | | Tyr | 92 L | O | 3.52 | |
| | | | Gln | 91 L | OE1 | 3.72 | |
| Asn | 97 I | CB | Tyr | 59 H | OH | 4.00 | |
| Asn | 97 I | C | Tyr | 59 H | OH | 3.85 | |
| Asn | 97 I | O | Tyr | 59 H | OH | 3.92 | * |
| Val | 98 I | N | Tyr | 59 H | OH | 3.92 | * |
| Val | 98 I | CG2 | Tyr | 59 H | OH | 3.90 | |
| | | | Tyr | 59 H | CE1 | 3.97 | |
| | | | Tyr | 59 H | CZ | 3.64 | |
| | | | Tyr | 59 H | CE2 | 3.79 | |
| | | | Trp | 95 L | CH2 | 4.00 | |
| Val | 98 I | O | Trp | 52 H | CH2 | 3.86 | |
| Ser | 99 I | CB | Tyr | 33 L | OH | 3.44 | |
| Ser | 99 I | OG | Tyr | 33 L | CZ | 3.61 | |
| | | | Tyr | 33 L | OH | 2.74 | *** |
| | | | Tyr | 33 L | CE1 | 3.65 | |
| Lys | 101 I | CB | Trp | 52 H | CH2 | 3.75 | |
| | | | Trp | 52 H | CZ2 | 3.93 | |
| Lys | 101 I | CG | Trp | 52 H | CE2 | 3.99 | |
| | | | Trp | 52 H | CH2 | 3.85 | |
| | | | Trp | 52 H | CZ2 | 3.56 | |
| Lys | 101 I | CD | Asp | 57 H | OD2 | 3.54 | |
| | | | Trp | 52 H | CE2 | 3.71 | |
| | | | Trp | 52 H | CD2 | 3.95 | |
| | | | Trp | 52 H | CH2 | 3.86 | |
| | | | Trp | 52 H | CZ2 | 3.70 | |
| Lys | 101 I | CE | Ser | 56 H | OG | 3.53 | |
| | | | Asp | 57 H | OD2 | 3.35 | |
| | | | Trp | 52 H | CG | 3.66 | |
| | | | Trp | 52 H | CD1 | 3.57 | |
| | | | Trp | 52 H | NE1 | 3.37 | |
| | | | Trp | 52 H | CE2 | 3.26 | |
| | | | Trp | 52 H | CD2 | 3.43 | |
| | | | Trp | 52 H | CZ2 | 3.79 | |
| Lys | 101 I | NZ | Ser | 56 H | OG | 3.23 | *** |
| | | | Asp | 57 H | OD2 | 3.34 | * |
| Lys | 102 I | CG | Trp | 52 H | CH2 | 3.82 | |
| Lys | 102 I | CD | Asp | 101 H | OD2 | 3.44 | |
| Lys | 102 I | CE | Asp | 101 H | OD2 | 3.56 | |
| | | | Tyr | 92 L | CZ | 3.79 | |
| | | | Tyr | 92 L | OH | 3.14 | |
| | | | Tyr | 92 L | CE2 | 3.48 | |
| Lys | 102 I | NZ | Asp | 101 H | CG | 3.73 | |
| | | | Asp | 101 H | OD2 | 2.90 | *** |
| | | | Tyr | 92 L | CZ | 3.84 | |
| | | | Tyr | 92 L | OH | 2.76 | *** |
| | | | Asp | 99 H | OD2 | 3.94 | * |
| | | | Asp | 101 H | CB | 3.74 | |
| Arg | 105 I | CD | Trp | 105 H | CE3 | 3.34 | |
| | | | Trp | 105 H | CZ3 | 3.70 | |
| Arg | 105 I | CZ | Ser | 102 H | O | 3.57 | |
| Arg | 105 I | NH1 | Ser | 103 H | CA | 3.89 | |
| | | | Ser | 103 H | C | 3.53 | |
| | | | Ser | 103 H | O | 3.44 | * |
| | | | Ser | 102 H | O | 3.08 | *** |
| | | | Asp | 104 H | C | 3.88 | |
| | | | Asp | 104 H | O | 3.95 | * |
| | | | Trp | 105 H | N | 3.88 | * |
| | | | Trp | 105 H | CA | 3.93 | |
| Arg | 105 I | NH2 | Asp | 101 H | CG | 3.38 | |
| | | | Asp | 101 H | OD1 | 2.95 | *** |
| | | | Asp | 101 H | OD2 | 3.25 | *** |
| | | | Ser | 102 H | O | 3.16 | *** |
| Lys | 106 I | O | Trp | 105 H | CE3 | 3.99 | |
| Pro | 107 I | CA | Trp | 105 H | CE3 | 3.80 | |
| | | | Trp | 105 H | CZ3 | 3.75 | |
| Pro | 108 I | CB | Tyr | 106 H | OH | 3.90 | |
| | | | Tyr | 106 H | CE2 | 3.69 | |
| Pro | 108 I | CG | Trp | 105 H | CD1 | 3.88 | |
| | | | Trp | 105 H | NE1 | 3.82 | |
| | | | Tyr | 106 H | CZ | 3.92 | |
| | | | Tyr | 106 H | OH | 3.37 | |
| | | | Tyr | 106 H | CE2 | 3.98 | |
| Pro | 108 I | CD | Trp | 105 H | NE1 | 3.91 | |
| | | | Trp | 105 H | CE2 | 3.75 | |
| | | | Trp | 105 H | CD2 | 3.93 | |

The determined epitopes for mAb 0114-0038, 0042 and 0044 determined by X-ray crystallography from the co-crystals of complexes of IL-21:Fab fragment 0114-0051, 0048 and 0050 respectively are similar to the determined epitope for mAb 0114-0005 to IL-21.

### Example 11

### Comparison of Interaction Kinetics for Anti hIL-21 Antibodies NNC 0114-0005, NNC 0114-0038, NNC 0114-0042 and NNC 0114-0044 to hIL-21 by Surface Plasmon Resonance (SPR)

All binding studies were performed on a Biacore T200 instrument that measures molecular interactions in real time through surface plasmon resonance. Experiments were run at 25° C. and the samples were stored at 10° C. in the sample compartment. The signal (RU, response units) reported by the Biacore is directly correlated to the mass on the individual sensor chip surfaces in four serial flow cells.

Anti-human Fc monoclonal from Biacore human Fc capture kit was immobilized onto flow cells of a CM4 sensor chip according to the manufacturer's instructions. The final immobilization level of capture antibody was approximately 2,000 RU in one experiment. Capture of the human anti-hIL21 antibody (1) NNCD 0114-0000-0005 (2) NNCD-0114-0000-0038 a Heavy-chain residue 31 Ser to Ala mutated form (H:S31A) of the anti hIL-21 human monoclonal antibody NNC 0114-0005 (3) NNC-0114-0042 a Light-chain residue 54 Ser to T mutated form (L:S53T) of the anti hIL-21 human monoclonal antibody NNC 0114-0005 or (4) NNC-0114-0044 a Light-chain residue 57 Thr to S mutated form (L:T57S) of the anti hIL-21 human monoclonal antibody NNC 0114-0005 was conducted by diluting each antibody to 0.06 μg/ml into running buffer (10 mM Hepes 0.3 M NaCl, 5 mM CaCl2, 0.05% surfactant P20, pH 8.0 containing 1 mg/ml BSA) and injected at 10 μl/min for 180s in one of flow cells 2-4, creating a reference surface in flow cell 1 with only anti-Fc antibody immobilized. This typically resulted in final capture levels of test antibodies of approximately 20 RU and Rmax values of analyte of 3-5 RU. Binding of hIL-21 protein was conducted by injecting analyte over all flow cells to allow for comparative analyses of binding to different captured anti-IL21 antibodies relative to binding to the reference flow cell. hIL-21 protein was diluted serially 1:3 to 0.008-2 nM into running buffer, injected at 100 μl/min for 210 s and allowed to dissociate for 600 or 14000 s. The CM4 surface was regenerated after each injection cycle of analyte via two injections of 3M MgCl₂ at 50 μl/min. This regeneration step removed the anti-IL-21 antibody and any bound IL-21 from the immobilized capture antibody surface, and allowed for the subsequent binding of the next interaction sample pair. The regeneration procedure did not remove the directly immobilized anti-Fc capture antibody from the chip surface.

In order to receive kinetic data, such as ka (association rate), kd (dissociation rate) and KD (equilibrium dissociation constant), data analysis was performed using the Biacore T200 evaluation software 1.0. No significant non-specific binding to the reference control surface was observed. Binding curves were processed by double referencing (subtraction of reference surface signals as well as blank buffer injections over captured anti-IL21 antibodies). This allowed correction for instrument noise, bulk shift and drift during sample injections.

Human IL-21 binds to NNCD 0114-0000-0005, 0038, 0042 and 0044 with an average KD of ≦1 pM, dissociation rates of 1 E-5 s⁻¹ and association rates of 3-4 E+7 (Ms)⁻¹. Results are based on two different experiments. Relative standard errors of parameters ka and kd were 0.2-2.1% in the individual experiments.

These data clearly demonstrates that the four different antibodies tested share similar binding properties to human IL-21

TABLE 13
Results from individual experiments of binding constants ka (association
rate), kd (dissociation rate) and KD (equilibrium dissociation constant)
for the interaction of human IL-21 to human and rat monoclonal antibodies
0114-0005, 0038, 0042 and 0044.

| | Exp | | | | RSE | RSE |
|---|---|---|---|---|---|---|
| Antibody | no | ka (1/Ms) | kd (1/s) | KD (M) | ka (%) | kd (%) |
| 0038 | 1 | 2.7E+07 | 1.2E−05 | 4.3E−13 | 0.4 | 0.3 |
| 0038 | 1 | 2.7E+07 | 1.1E−05 | 4.2E−13 | 0.7 | 0.5 |
| 0038 | 2 | 7.5E+07 | 6.8E−06 | 9.0E−14 | 2.1 | 0.7 |
| 0042 | 1 | 1.8E+07 | 2.8E−05 | 1.5E−12 | 0.7 | 0.2 |
| 0042 | 2 | 3.5E+07 | 2.1E−05 | 6.1E−13 | 0.7 | 0.4 |
| 0044 | 1 | 2.2E+07 | 1.0E−05 | 4.6E−13 | 0.4 | 0.3 |
| 0044 | 2 | 5.4E+07 | 1.3E−05 | 2.4E−13 | 2.0 | 0.7 |
| 0005 | 1 | 1.6E+07 | 9.5E−06 | 5.8E−13 | 0.4 | 0.3 |
| 0005 | 1 | 1.5E+07 | 1.1E−05 | 7.2E−13 | 0.4 | 0.3 |
| 0005 | 2 | 6.6E+07 | 1.1E−05 | 1.7E−13 | 1.5 | 0.4 |

### Example 12

### B Cell Proliferation and Maturation Assays of Anti hIL-21 Antibodies NNC 0114-0005, NNC 0114-0038, NNC 0114-0039 and NNC 0114-0040, NNC 0114-0041 and NNC 0114-0042, NNC 0114-0043, NNC 0114-0044

The neutralising potential of seven anti-IL-21 antibodies was compared. The antibodies were all based on the reference antibody 0114-0000-0005 but each of the antibodies contained a point mutation making it differing by a single amino acid (Table 6 in Example 3). The antibodies were tested for their ability to neutralise the recombinant human IL-21 in the B cell proliferation assay. The anti-IL-21 0114-0000-0006 and 0114-0000-0019 was included as a neutralising and partly-neutralising control, respectively.

mAb 0114-0000-0005 and mAb 0114-0000-0006 are recombinantly produced antibodies, identical in amino acid sequence, but produced in different CHO cell lines. MAb 0114-0000-0005 was produced in a stable CHO DXB-11 cell line and mAb 0114-0000-0006 was produced in a stable CHO-K1SV cell line.

Human B cells were isolated from 4 individual donors. The B cells were plated at 50.000 cells per well in a 96-well U-bottom tissue culture plate. Cells were treated with 0.1 μg/ml anti-CD40 (R&D Systems), 50 ng/ml (3.21 nM) recombinant human IL-21. Due to the number of antibodies tested it was not possible to test all antibodies with cells isolated from same donor. Consequently, antibodies -0038, -0039, -0040 and -0041 were tested with B cells from donor 1 and 3 and antibodies -0042, -0043 and -0044 were tested with B cells from donor 2 and 4. All 4 donors were tested with -0006 and -0015 as controls for donor variation. Antibody 0114-0000-0019 was tested with donor 1 and 3. The cells were incubated for 3 days at 37° C. and 5% CO₂ in a humidified incubator. The antibodies were titrated and after three days, the cells were pulsed with 1 μCi/well of [³H]-Thymidine (Perkin Elmer Life Sciences) for the last 20 hours. The cells were harvested onto UniFilter-96 GF/C filter plates (Packard Instruments, Perkin Elmer) and the amount of [³H]-thymidine incorporation was quantified using a TopCount NXT (Perkin Elmer). The inhibitive concentration of anti IL-21 mAb required for reducing proliferation by 50% (IC₅₀) was calculated using the GraphPad Prism v5.0 software (GraphPad Inc.) and the sigmoidal dose-response (variable slope, 4-parameters) equation.

The IC₅₀ for the 7 mutated antibodies were all found to be very similar with IC₅₀ values in the low nanomolar range and comparable to the IC₅₀ for NNC-0114-0005.

TABLE 14
IC₅₀

| | IC₅₀ (nM) | IC₅₀ (nM) | IC₅₀ (nM) | IC₅₀ (nM) |
|---|---|---|---|---|
| | Donor 1 | Donor 3 | Donor 2 | Donor 4 |
| 0114-0000-0038 | 1.18 | 1.21 | | |
| 0114-0000-0039 | 0.78 | 0.92 | | |
| 0114-0000-0040 | 0.93 | 1.00 | | |
| 0114-0000-0041 | 1.15 | 1.12 | | |
| 0114-0000-0042 | | | 1.23 | 1.4 |
| 0114-0000-0043 | | | 0.3 | 1.35 |
| 0114-0000-0044 | | | 0.8 | 1.17 |
| 0114-0000-0006 | 0.82 | 0.57 | 0.83 | 1.33 |
| 0114-0000-0015 | 1.23 | 1.13 | 0.86 | 0.91 |
| 0114-0000-0019 | NA | NA | | |

### Example 13

### Bioactivity in NK-92 Assay of Anti hIL-21 Antibodies NNC 0114-0006, NNC 0114-0038, NNC 0114-0039 and NNC 0114-0040, NNC 0114-0041 and NNC 0114-0042, NNC 0114-0043, NNC 0114-0044

The antibodies were tested for their ability to neutralise the recombinant human IL-21 in the NK-cell based bioassay. The anti-IL-21 mAb (NNC0114-0000-0006) was included as reference material.

The NK-cell based bioassay was used for in vitro determination of the bioactivity of anti-IL-21 antibodies. The NK-92 cell line (ATCC/LGC Promochem) is a human suspension lymphoblast derived from peripheral blood mononuclear cells. Cells express the IL-21 receptor endogenously and are dependent on IL-2 or IL-21 for cell proliferation. The neutralization of IL-21 by anti-IL-21 is measured by growth inhibition via addition of alamarBlue® (a cell viability indicator).

During maintenance the NK-92 cells were kept proliferating by addition of IL-2. For assay, NK-92 cells were washed and plated out in 96 well plates (Matrix Technology) at a density of 1.6×10⁵ cells/ml (equal to 12,800 cells per well). The cells were stimulated with recombinant human IL-21 at a fixed concentration of 5431 pg/ml. Serial dilutions of Anti-IL-21 antibodies prepared in assay media, ranging from 0-12,800 pg/ml, was added in triplicates in three different positions in the 96-well plate. The cells were incubated for 3 days at 37° C. and 5% CO₂ in a humidified incubator. On day three 10 μl alamarBlue® (Biosource) was added and fluorescence was measured after 5 hours of incubation on a Synergy instrument (Bio Tek).

Data was analyzed in BioCalc (MicroLex) in a four-parameter logistic curve model. Results are given as percentage (%) of reference material (0114-0000-0006), based on two independent setups each with a triple determination.

The bioactivity measured for the 7 mutated antibodies were all found to be very similar when compared relative to the bioactivity of the reference material (NNC0114-0000-0006).

TABLE 15
Bioactivity

| | Bioactivity as % of RM |
|---|---|
| | (NCC0114-0000-0006) |
| 0114-0000-0038 | 89.2 |
| 0114-0000-0039 | 95.2 |
| 0114-0000-0040 | 92.9 |
| 0114-0000-0041 | 83.9 |
| 0114-0000-0042 | 96.5 |
| 0114-0000-0043 | 89.8 |
| 0114-0000-0044 | 77.8 |
| 0114-0000-0015-2A | 111.9 |

### Example 14

### Generation of Expression Vectors for Transient Expression of Anti-IL21 mAb 0006 and Fab Fragment

To enable epitope mapping and binding analyses, a series of CMV promotor-based expression vectors (pTT vectors) were generated for transient expression of mAb 0006 variants in the HEK293-6E EBNA-based expression system developed by Yves Durocher (Durocher et al. Nucleic Acid Research, 2002). In addition to the CMV promotor, the vectors contain a pMB1 origin, an EBV origin and the Amp resistance gene.

The region corresponding to the mAb 0006 VH domain was cloned from the original mAb 0005 expression vector into a linearized pTT-based vector containing the sequence of the engineered human IgG1.1 (containing 5 amino acid substitutions: L234A, L235E, G237A, A330S, P331S)CH domain using standard PCR and restriction-based cloning methods. Vector constructs were transformed into E. coli for selection. The sequence of the final construct was verified by DNA sequencing.

A pTT-based vector was also generated for transient expression of the mAb 0006 Fab fragment. The region corresponding to aa residues 1-253 was PCR amplified from the mAb 0006 expression vector with a generic vector specific primer and a primer containing a premature stop codon in the HC hinge region and a terminal restriction site adaptor for cloning purposes. The PCR generated insert was cloned into a linearized pTT vector by restriction-based cloning and transformed into E. coli for selection. The sequence of the final construct was verified by DNA sequencing.

The region corresponding to the mAb 0006 VL domain was cloned from the original mAb 0005 expression vector into a linearized pTT-based vector containing the sequence for a human kappa CL domain using standard PCR and restriction-based cloning methods. Vectors constructs were transformed into E. coli for selection. The sequence of the final construct was verified by DNA sequencing.

### Recombinant Expression of mAb Variants:

Variants of mAb 0006 including Fab fragments were expressed by co-transfection of HEK293-6E cells with pTT-based HC and LC vectors according to the generic antibody expression protocol listed below.

### Cell Maintenance:

HEK293-6E cells were grown in suspension in FreeStyle™ 293 expression medium (Gibco) supplemented with 25 μg/ml Geneticin (Gibco), 0.1% v/v of the surfactant Pluronic F-68 (Gibco) & 1% v/v Penicillin-Streptomycin (Gibco). Cells were cultured in Erlenmeyer shaker flasks in shaker incubators at 37° C., 8% CO₂ and 125 rpm and maintained at cell densities between 0.1−1.5×10⁶ cells/ml.

### DNA Transfection:

The cell density of cultures used for transfection was 0.9−2.0×10⁶ cells/ml.
A mix of 0.5 μg LC vector DNA+0.5 μg HC vector DNA was used per ml cell culture.
The DNA was diluted in Opti-MEM media (Gibco) 30 μl media/μg DNA, mixed and incubated at room temperature (23-25° C.) for 5 min.
293Fectin™ (Invitrogen) was used as transfection reagent at a concentration of 1 μl per μg DNA.
The 293Fectin™ was diluted 30× in Opti-MEM media (Gibco), mixed and incubated at room temperature (23-25° C.) for 5 min.
The DNA and 293Fectin solutions were mixed and left to incubate at room temperature (23-25° C.) for 25 min.
The DNA-293Fectin mix was then added directly to the cell culture.
The transfected cell culture was transferred to a shaker incubator at 37° C., 8% CO₂ and 125 rpm.
5 days post transfection, cell culture supernatants were harvested by centrifugation, followed by filtration through a 0.22 μm PES filter (Corning).
Quantitative analysis of antibody production was performed by Biolayer Interferometry directly on clarified cell culture supernatants using the ForteBio Octet system or by SDS-PAGE analysis.

### Example 15

### Site-Directed Mutagenesis of Anti-IL21 mAb 0006

Site-directed mutagenesis was performed to generate variants of anti-IL21 mAb 0006. Mutations were introduced in the mAb 0006 HC or LC by two different methods:
1) The QuickChange® Site-Directed mutagenesis kit from Stratagene and specific mutagenic primers were used to introduce point mutations. The kit was used according to the manufacturer's protocol.
2) Standard 2-step overlapping PCR with mutagenic primers was also used as an alternative to introduce point mutation

The pTT-based expression plasmids for mAb 0006 LC and HC described in example 14 were used as templates for the mutagenesis. The sequences of all final constructs were verified by DNA sequencing.

The plasmid for expression of the truncated version of HC mutant S31A (for Fab fragment expression) was generated by replacing the VH domain in the WT Fab expression vector (described previously) with the S31A mutant VH domain. Domain swapping was done by standard restriction-based cloning methods. Vectors constructs were transformed into E. coli for selection. The sequence of the final construct was verified by DNA sequencing.

TABLE 16
Variants of mAb 0006

| CDR | # | Chain | Name | Mutations |
|---|---|---|---|---|
| H1 | 31 | H | SER | S31A |
| L2 | 53 | L | SER | S53A |
| L2 | 53 | L | SER | S53T |
| L2 | 54 | L | SER | S54A |
| L2 | 54 | L | SER | S54T |
| L2 | 55 | L | ARG | R55K |
| L2 | 57 | L | THR | T57S |

To express mAb 0006 mutants, HEK293-6E cells were co-transfected with LC plasmids (WT or mutants) and HC plasmids (WT or mutant). To express mAb 0006 Fab fragment, HEK293-6E cells were co-transfected with LC plasmids (WT or mutants) and truncated HC plasmids (WT or mutant). The LC:HC combinations are listed in table 17 below.

TABLE 17
LC:HC combinations

| LC | | HC | Fab | | | |
|---|---|---|---|---|---|---|
| plasmid | LC | plasmid | plasmid | HC | | |
| ID | Mutation | ID | ID | mutation | mAb ID | Fab ID |
| 353 | WT | 354 | 398 | WT | 0006 | 0009 |
| 353 | WT | 479 | 520 | S31A | 0038 | 0051 |
| 480 | S53A | 354 | 398 | WT | 0039 | 0045 |
| 481 | S53T | 354 | 398 | WT | 0040 | 0046 |
| 482 | S54A | 354 | 398 | WT | 0041 | 0047 |
| 483 | S54T | 354 | 398 | WT | 0042 | 0048 |
| 484 | R55K | 354 | 398 | WT | 0043 | 0049 |
| 485 | T57S | 354 | 398 | WT | 0044 | 0050 |

### Purification of mAb and Fab Fragment Variants:

Mab 0006 variants were purified by standard affinity chromatography using MabSelectSuRe resin from GE Healthcare. The purified antibodies were buffer exchanged to PBS buffer pH7.2.

Fab fragments were purified by standard affinity chromatography using KappaSelect resin from GE Healthcare. The purified Fab fragments were buffer exchanged to PBS buffer pH7.2.

Quality assessment and concentration determination was done by SEC-HPLC

### Example 16

### Epitope Mapping by HX-MS of mAbs 0114-0005 and 0114-0038, -0039, -0040, -0041 and -0042 on hIL-21

### Materials

Protein Batches Used were:

hIL-21: human recombinant IL-21, corresponding to residues 30-162 and with an N-terminal Methionine as residue 29.

mAb: 0114-0005

mAb variants of 0005: 0114-0038, -0039, -0040, -0041 and -0042. (see table 17 in example 15 for description of the variants)

All proteins were buffer exchanged into PBS pH 7.4 before experiments.

### Methods: HX-MS Experiments

### Instrumentation and Data Recording

The HX experiments were automated by a Leap robot (H/D-x PAL; Leap Technologies Inc.) operated by the LeapShell software (Leap Technologies Inc.), which performed initiation of the deuterium exchange reaction, reaction time control, quench reaction, injection onto the UPLC system and digestion time control. The Leap robot was equipped with two temperature controlled stacks maintained at 20° C. for buffer storage and HX reactions and maintained at 2° C. for storage of protein and quench solution, respectively. The Leap robot furthermore contained a cooled Trio VS unit (Leap Technologies Inc.) holding the pre- and analytical columns, and the LC tubing and switching valves at 1° C. The switching valves of the Trio VS unit have been upgraded from HPLC to Microbore UHPLC switch valves (Cheminert, VICI AG). For the inline pepsin digestion, 100 μL quenched sample containing 200 pmol hIL-21 was loaded and passed over a Poroszyme® Immobilized Pepsin Cartridge (2.1×30 mm (Applied Biosystems)) placed at 20° C. using a isocratic flow rate of 200 μL/min (0.1% formic acid:CH₃CN 95:5). The resulting peptides were trapped and desalted on a VanGuard pre-column BEH C18 1.7 μm (2.1×5 mm (Waters Inc.)). Subsequently, the valves were switched to place the pre-column inline with the analytical column, UPLC-BEH C18 1.7 μm (2.1×100 mm (Waters Inc.)), and the peptides separated using a 9 min gradient of 15-35% B delivered at 200 μl/min from an AQUITY UPLC system (Waters Inc.). The mobile phases consisted of A: 0.1% formic acid and B: 0.1% formic acid in CH₃CN. The ESI MS data, and the separate data dependent MS/MS acquisitions (CID) and elevated energy (MS^{E}) experiments were acquired in positive ion mode using a Q-TOF Premier MS (Waters Inc.). Leucine-enkephalin was used as the lock mass ([M+H]⁺ ion at m/z 556.2771) and data was collected in continuum mode (For further description of the set-up, see Andersen and Faber, Int. J. Mass Spec., 302, 139-148 (2011)).

### Data Analysis

Peptic peptides were identified in separate experiments using standard CID MS/MS or MS^{E} methods (Waters Inc.). MS^{E} data were processed using BiopharmaLynx 1.2 (version 017). CID data-dependent MS/MS acquisition was analyzed using the MassLynx software and in-house MASCOT database.

The HX-MS raw data files were processed in the DynamX software (Waters Inc.). DynamX performs the lock mass-correction and deuterium incorporation determination, i.e., centroid determination of deuterated peptides.

### Epitope Mapping Experiment

Amide hydrogen/deuterium exchange (HX) was initiated by a 16-fold dilution of hIL-21 in the presence or absence of 0114-0005 or 0114-0038, -0039, -0040, -0041 or -0042 into the corresponding deuterated buffer (i.e. PBS prepared in D₂O, 96% D₂O final, pH 7.4 (uncorrected value)). All HX reactions were carried out at 20° C. and contained 4 μM hIL-21 in the absence or presence of 2.4 μM mAb thus giving a 1.2 fold molar excess of mAb binding sites. At appropriate time intervals ranging from 10 sec to 28800 sec, 50 μl aliquots of the HX reaction were quenched by 50 μl ice-cold quenching buffer (1.35M TCEP) resulting in a final pH of 2.5 (uncorrected value).

### Results and Discussion

Epitope Mapping of 0114-0005, -0038, -0039, -0040, -0041 and -0042

The epitope mapping of mAb 0005 on hIL-21 has already been described in example 7. However, mAb 0005 was also included in the present experiments for reference. The HX time-course of 29 peptides, covering 91% of the primary sequence of hIL-21

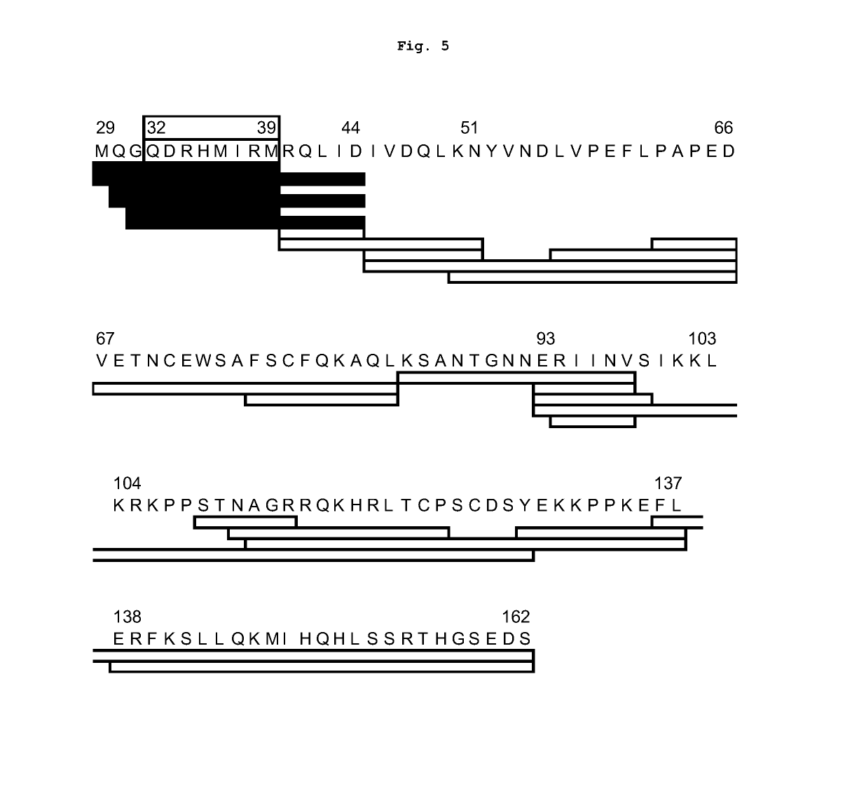

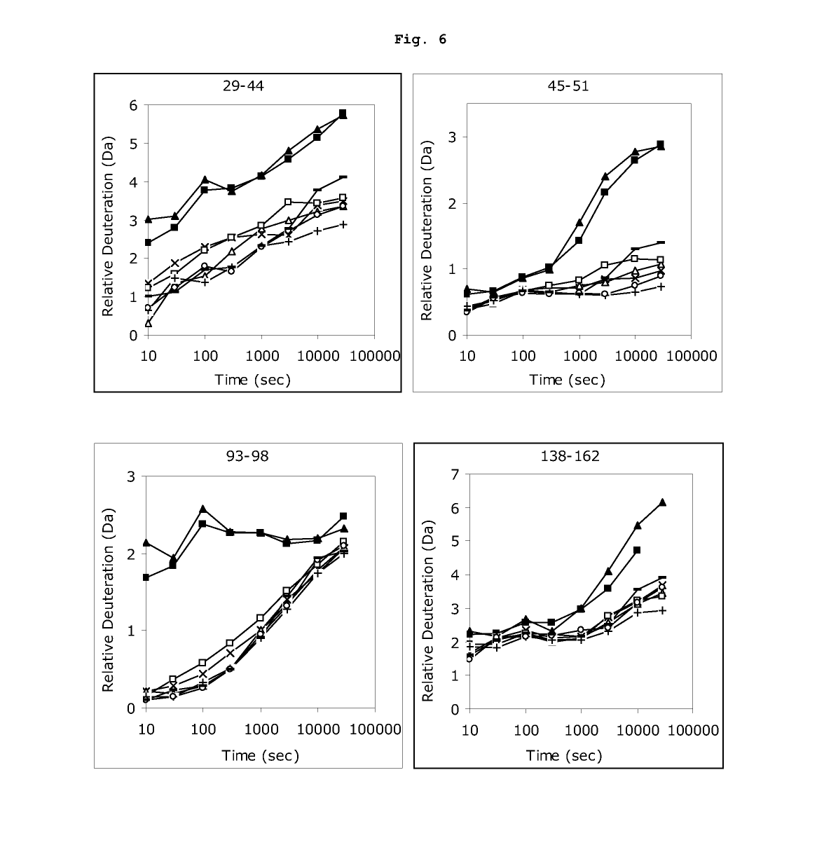

were monitored in the absence or presence of 0114-0005, -0038, -0039, -0040, -0041 or -0042 for 10 to 28800 sec. However region 40-51, a central part of helix A, does not contain fast exchanging amide hydrogens (FIG. 6). Thus, no epitope information is gained from this region by HX-MS.

### Epitope Mapping

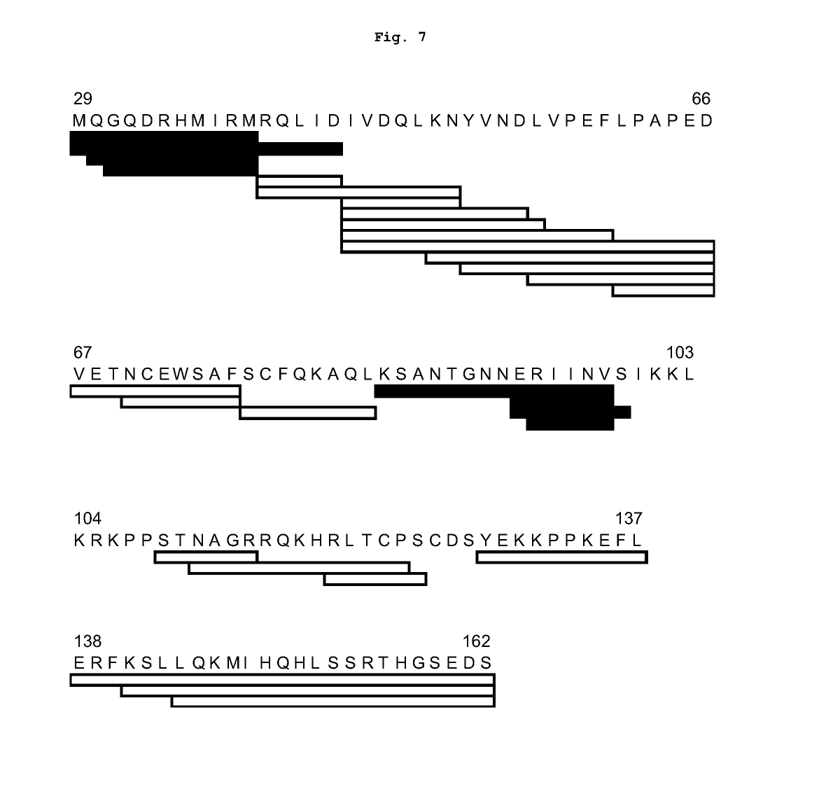

The observed exchange pattern in the early timepoints (<300 sec) in the presence or absence of 0114-0005, -0038, -0039, -0040, -0041 or -0042 can be divided into two different groups: One group of peptides display an exchange pattern that is unaffected by the binding of these mAbs in the early timepoints. In contrast, another group of peptides in hIL-21 show protection from exchange upon 0114-0005, -0038, -0039, -0040, -0041 or -0042 binding (FIGS. 6 and 7). For example at 100 sec exchange with D₂O, approximately 2 amides are protected from exchange in the region E93-V98 upon binding of any of the 0114-0005, -0038, -0039, -0040, -0041 or -0042 mAbs (FIG. 6). Interestingly, the same group of hIL-21 derived peptides were affected by binding of these mAbs thus the epitopes for 0114-0038, -0039, -0040, -0041 and -0042 appear identical to the epitope for 0114-0005 as determined in example 7.

0114-0005, -0038, -0039, -0040, -0041 and -0042 Stabilize the Entire hIL-21 Structure

Apart from epitope effects; the HX time-course of all 29 peptides, covering 91% of the primary sequence of hIL-21 also displayed additional very interesting and surprising features. Binding of 0114-0005 or any of the −0038, -0039, -0040, -0041 and -0042 mAbs to hIL-21 resulted in a marked reduction of deuterium exchange of hIL-21 in the late time-points of HX exchange, i.e. above 300 sec (see for example I45-N51 and E138-S162 in FIG. 6). Effects observed late in the time course are related to slow exchanging amide hydrogens and thus related to the structural core of the protein. In contrast, early effects are related to the surface exposed amide hydrogens. Thus, epitope effects appear in the early time points whereas structural stabilization effects will manifest as exchange reduction in late time points (Garcia, Pantazatos and Villareal, Assay and Drug Dev. Tech. 2, 81 (2004); Mandell, Falick and Komives, Proc. Natl. Acad. Sci. USA, 95, 14705 (1998); Andersen and Faber, Int. J. Mass Spec., 302, 139-148 (2011). While minor structural stabilization effects normally can occur locally around a binding site, the effects observed here in hIL-21 upon binding of 0114-0005, -0038, -0039, -0040, -0041 or -0042 to hIL-21 are highly remarkable: The 145-N51 and E138-S162 shown in FIG. 6 are only examples. The structural stabilization effects are observed in every part of the entire hIL-21 molecule. Thus, upon binding of 0114-0005, -0038, -0039, -0040, -0041 or -0042 to hIL-21 every region of hIL-21 is stabilized and thus also including all regions very distal to the epitope. Furthermore, the magnitude of the stabilization effects are unusually large as shown in FIG. 6 with two and three amide hydrogens protected from exchange after 28800 sec in regions 145-N51 and E138-S162, respectively.

### CONCLUSION

Upon binding of either 0114-0005, -0038, -0039, -0040, -0041 or -0042 all regions of hIL-21 showed similar responses. The same group of peptides were affected by mAb binding in the early time-points thus the epitopes for 0114-0038, -0039, -0040, -0041 and -0042 appear identical to the epitope for 0114-0005 determined in example 7. Furthermore all regions of hIL-21 displayed structural stabilization effects upon binding of either mAb, thus 0114-0038, -0039, -0040, -0041 and -0042 all stabilize hIL-21 similarly to 0114-0005.

### ABBREVIATIONS

Aa: amino acid
mAb: monoclonal antibody
HC: heavy chain
LC: light chain
VH: variable domain—heavy chain
VL: variable domain—light chain
CH: constant region—heavy chain
CL: constant region—light chain
PCR: polymerase chain reaction
WT: wild type

### REFERENCES

[1] Ursby, T., Mammen, C. B., Cerenius, Y., Svensson, C., Sommarin, B., Fodje, M. N., Kvick, A., Logan, D. T., Als-Nielsen, J., Thunnissen, M. M. G. M., Larsen, S., & Liljas, A. The New Macromolecular Crystallography Stations At MAX-lab: The MAD Station. AIP Conference Proceedings 705, 1241-1246. 2004.
[2] Kabsch, W. (1993) Automatic processing of rotation diffraction data from crystals of initially unknown symmetry and cell constants. J. Appl. Crystallogr. 26, 795-800.
[3] Evans, P. (2006) Scaling and assessment of data quality. Acta crystallographica. Section D, Biological crystallography 62, 72-82.
[4] Mccoy, A. J., Grosse-Kunstleve, R. W., Storoni, L. C., & Read, R. J. (2005) Likelihood-enhanced fast translation functions. Acta Crystallographica Section D Biological Crystallography 61, 458-464.
[5] Mccoy, A. J., Grosse-Kunstleve, R. W., Adams, P. D., Winn, M. D., Storoni, L. C., & Read, R. J. (2007) Phaser crystallographic software. J. Appl. Crystallogr. 40, 658-674.
[6] Bailey, S. (1994) The ccp4 suite—programs for protein crystallography. Acta Crystallogr. Sect. D-Biol. Crystallogr. 50, 760-763.
[7] Berman, H. M., Westbrook, J., Feng, Z., Gilliland, G., Bhat, T. N., Weissig, H., Shindyalov, I. N., & Bourne, P. E. (2000) The Protein Data Bank. Nucleic Acids Res. 28, 235-242.
[8] Niemi, M. H., Takkinen, K., Amundsen, L. K., Soderlund, H., Rouvinen, J., & Hoyhtyä, M. (2010) The testosterone binding mechanism of an antibody derived from a naïve human scFv library. J. Mol. Recognit. n/a.
[9] Perrakis, A., Morris, R., & Lamzin, V. S. (1999) Automated protein model building combined with iterative structure refinement. Nat Struct Biol 6, 458-463.
[10] Murshudov, G. N., Vagin, A. A., & Dodson, E. J. (1997) Refinement of macromolecular structures by the maximum-likelihood method. Acta Crystallogr. Sect. D-Biol. Crystallogr. 53, 240-255.
[11] Afonine, P. V., Grosse-Kunstleve, R. W., & Adams, P. D. Contribution 8. CCP4 Newsletter [42]. 2005.
[12] Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., McCoy, A. J., Moriarty, N. W., Oeffner, R., Read, R. J., Richardson, D. C., Richardson, J. S., Terwilliger, T. C., & Zwart, P. H. (2010) PHENIX: a comprehensive Python-based system for macromolecular structure solution. Acta Cryst. D 66, 213-221.
[13] Emsley, P. & Cowtan, K. (2004) Coot: model-building tools for molecular graphics. Acta Crystallogr. Sect. D-Biol. Crystallogr. 60, 2126-2132.
[14] Bondensgaard, K., Breinholt, J., Madsen, D., Omkvist, D. H., Kang, L., Worsaae, A., Becker, P., Schiodt, C. B., & Hjorth, S. A. (2007) The existence of multiple conformers of interleukin-21 directs engineering of a superpotent analogue. J. Biol. Chem. 282, 23326-23336.
[15] Terwilliger, T. C. (2000) Maximum-likelihood density modification. Acta Crystallogr. Sect. D-Biol. Crystallogr. 56, 965-972.

All publications mentioned in the above specification are herein incorporated by reference. Various modifications and variations of the described aspects and embodiments of the present invention will be apparent to those skilled in the art without departing from the scope of the present invention.

1. A ligand which binds to a discontinuous epitope on IL-21, wherein said epitope comprises at least one of amino acids I37 to Y52 and at least one of amino acids N92 to P108 of IL-21, as set forth in SEQ ID NO:1, provided that the ligand is not: (i) naturally occurring IL-21Rα (SEQ ID NO: 14), (ii) the monoclonal antibody NNC 0114-0005, the light and heavy chains of which are set forth in SEQ ID NO: 10 and SEQ ID NO: 11 respectively, and (iii) the monoclonal antibody NNC 0114-0015, the light and heavy chains of which are set forth in SEQ ID NO: 12 and SEQ ID NO: 13 respectively.

2. A ligand according to claim 1, wherein said ligand binds to an epitope comprising amino acids I37 to Y52 and N92 to P108 of IL-21 as set forth in SEQ ID NO: 1.

3. A ligand according to claim 1, wherein said ligand is an antibody.

4. An antibody according to claim 3, wherein said antibody comprises a light chain comprising at least one of CDR1, CDR2 or CDR3 as set forth in SEQ ID NO: 10, and a heavy chain comprising at least one of CDR1, CDR2 or CDR3 as set forth in SEQ ID NO: 11.

5. An antibody according to claim 4, wherein said antibody comprises a light chain comprising at least one of CDR1 and CDR3 as set forth in SEQ ID NO: 10, and a heavy chain comprising at least one of CDR2 and CDR3 as set forth in SEQ ID NO: 11

6. A ligand which binds to IL-21 at the binding interface between IL-21 and IL-21Rα, wherein said ligand binds to an epitope which includes at least one of R34, R38, Q41, R105, and K102 in the amino acid sequence of IL-21 set forth in SEQ ID NO: 1, provided that the ligand is not: (i) naturally occurring IL-21Rα (SEQ ID NO: 14), (ii) the monoclonal antibody NNC 0114-0005, the light and heavy chains of which are set forth in SEQ ID NO: 10 and SEQ ID NO: 11 respectively, and (iii) the monoclonal antibody NNC 0114-0015, the light and heavy chains of which are set forth in SEQ ID NO: 12 and SEQ ID NO: 13 respectively.

7. A ligand according to claim 6, wherein said ligand binds to R34, R38, Q41, R105, and K102 in the sequence of IL-21 set forth in SEQ ID NO: 1.

8. A ligand according to claim 1, wherein said ligand interferes with the binding of IL-21 to IL-21Rα.

9. A ligand according to claim 1, wherein the KD of the interaction of human IL-21 with the ligand is 10⁻¹² (M) or less.

10. A ligand according to claim 1, wherein said ligand is an antibody, and wherein said antibody comprises the CDR3 amino acid sequence as set forth in SEQ ID NO: 10 and the CDR3 amino acid sequence as set forth in SEQ ID NO: 11.

11. A ligand according to claim 1, wherein said ligand is an antibody, wherein the KD of the interaction of human IL-21 with said antibody is 10⁻¹² (M) or less, wherein said antibody binds to R34, R38, Q41, R105, and K102 in the sequence of IL-21 set forth in SEQ ID NO: 1, and wherein said antibody competes with IL-21 for binding to IL-21R.

12. A pharmaceutical composition comprising a ligand according to claim 1 and optionally one or more pharmaceutically acceptable excipients.

13. (canceled)

14. A method of treating an immunological disorder, wherein said method comprises administering to a person in need thereof an appropriate dose of a ligand according to claim 1.

15. A method of treating an immunological disorder, wherein said method comprises administering to a person in need thereof an appropriate dose of a ligand according to claim 1, wherein said ligand is used for reducing B cell differentiation in the treatment of autoimmune diseases

16. A ligand according to claim 1, wherein said ligand is an antibody that is a variant of the monoclonal antibody NNC 0114-0005, the light and heavy chains thereof which are set forth in SEQ ID NO: 10 and SEQ ID NO: 11 respectively, wherein said ligand comprises one or more mutations in the CDR sequences of SEQ ID NO: 10 and/or SEQ ID NO: 11, wherein said mutations are selected from one or more from the list consisting of: CDR H1 S31A, CDR H2 Y53F, CDR H2 A61S, CDR H2 S63T, CDR H2S63A, CDR H2 K65R, CDR L1 R24K, CDR L1 S26T, CDR L1 S31T, CDR L1 S31A, CDR L2 S53T, CDR L2 S52A, CDR L2 S54A, CDR L2 S54T, and CDR L2 R55K.

17. A ligand according to claim 7, wherein said ligand interferes with the binding of IL-21 to IL-21Rα.

18. A ligand according to claim 6, wherein the KD of the interaction of human IL-21 with the ligand is 10⁻¹² (M) or less.

19. A ligand according to claim 6, wherein said ligand is an antibody, and wherein said antibody comprises the CDR3 amino acid sequence as set forth in SEQ ID NO: 10 and the CDR3 amino acid sequence as set forth in SEQ ID NO: 11.

20. A ligand according to claim 6, wherein said ligand is an antibody, wherein the KD of the interaction of human IL-21 with said antibody is 10⁻¹² (M) or less, wherein said antibody binds to R34, R38, Q41, R105, and K102 in the sequence of IL-21 set forth in SEQ ID NO: 1, and wherein said antibody competes with IL-21 for binding to IL-21R.

21. A pharmaceutical composition comprising a ligand according to claim 6 and optionally one or more pharmaceutically acceptable excipients.

22. A method of treating an immunological disorder, wherein said method comprises administering to a person in need thereof an appropriate dose of a ligand according to claim 6.

23. A method of treating an immunological disorder, wherein said method comprises administering to a person in need thereof an appropriate dose of a ligand according to claim 6, wherein said ligand is used for reducing B cell differentiation in the treatment of autoimmune diseases

24. A ligand according to claim 6, wherein said ligand is an antibody that is a variant of the monoclonal antibody NNC 0114-0005, the light and heavy chains thereof which are set forth in SEQ ID NO: 10 and SEQ ID NO: 11 respectively, wherein said ligand comprises one or more mutations in the CDR sequences of SEQ ID NO: 10 and/or SEQ ID NO: 11, wherein said mutations are selected from one or more from the list consisting of: CDR H1 S31A, CDR H2 Y53F, CDR H2 A61S, CDR H2 S63T, CDR H2S63A, CDR H2 K65R, CDR L1 R24K, CDR L1 S26T, CDR L1 S31T, CDR L1 S31A, CDR L2 S53T, CDR L2 S52A, CDR L2 S54A, CDR L2 S54T, and CDR L2 R55K.


SEQUENCE LISTING (17 sequences)

SEQ ID NO: 1 - protein, 162 aa, homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - protein, 19 aa, homo sapiens
RHMIRMRQLIDIVDQLKNY

SEQ ID NO: 3 - protein, 11 aa, homo sapiens
EWSAFSCFQKA

SEQ ID NO: 4 - protein, 11 aa, homo sapiens
ERIINVSIKKL

SEQ ID NO: 5 - protein, 20 aa, homo sapiens
PKEFLERFKSLLQKMIHQHL

SEQ ID NO: 6 - protein, 16 aa, homo sapiens
IRMRQLIDIVDQLKNY

SEQ ID NO: 7 - protein, 17 aa, homo sapiens
NERIINVSIKKLKRKPP

SEQ ID NO: 8 - protein, 18 aa, homo sapiens
HMIRMRQLIDIVDQLKNY

SEQ ID NO: 9 - protein, 15 aa, homo sapiens
IINVSIKKLKRKPPS

SEQ ID NO: 10 - protein, 213 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 456 aa, homo sapiens
QVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 213 aa, homo sapiens; identical to SEQ ID NO: 10

SEQ ID NO: 13 - protein, 456 aa, homo sapiens
QVQLVESGGGVVQPGRSLRLSCAASGFTFSTYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 538 aa, homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 15 - protein, 456 aa, homo sapiens
QVQLVESGGGVVQPGRSLRLSCAASGFTFSTYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 213 aa, homo sapiens; identical to SEQ ID NO: 10

SEQ ID NO: 17 - protein, 456 aa, homo sapiens; identical to SEQ ID NO: 11